SEQUENCE LISTING (57 sequences)

SEQ ID NO: 1 - protein, 255 aa, Homo sapiens
MTMTTMPESLNSPVSGKAVFMEFGPPNQQMSPSPMSHGHYSMHCLHSAGHSQPDGAYSSASSFSRPLGYPYVNSVSSHASSPYISSVQSYPGSASLAQSRLEDPGADSEKSTVVEGGEVRFNGKGKKIRKPRTIYSSLQLQALNRRFQQTQYLALPERAELAASLGLTQTQVKIWFQNKRSKFKKLMKQGGAALEGSALANGRALSAGSPPVPPGWNPNSSSGKGSGGNAGSYIPSYTSWYPSAHQEAMQQPQLM

SEQ ID NO: 2 - DNA, 2403 bp, Homo sapiens
aagctttgaaccgagtttggggagctcagcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggggcggactcggagaagagcacggtggtggaaggcggtgaagtgcgcttcaatggcaagggaaaaaagatccgtaaacccaggacgatttattccagtttgcagttgcaggctttgaaccggaggttccagcaaactcagtacctagctctgccggagagggcggagctcgcggcctctttgggactcacacagactcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgggtcctgccgggctgaccatctccggatcctgggacactctgcctgaccatctgtgtagctggtgtgggaatctgggggcattggagggagggggttttatttattgagaaatggacttcgcctgaggctgtttgccaattcagggttctgctgggcgcaaggaacgcactgttcaaacgcactgtttactttaagcgcacggggagaaacgaataaggaggacgtggtgatttttaatttatacagtaacttttgtacttctctggtatggagagtttggagccgaatgatttgcattttttacatgtccgacattatttaataaataatttttaaaagaaaagaacgataaatgaagccaacatgattttctcatttcgggaggaactctgttgcttcgcctggacaagaaggaaaatgctgatttcctccttgggtagaaagagggagcgagggcaaatggggagtagagagaaaacaggcgagaacaagcactctaattccagtgggctttaaaataagacaaaatcagctttacaacaatccctagaggctcgaccacagaataatgccagtcaccaccctgaacgcacaatctccagtgcaggatctaatgactgtacatattattgttattattattattgttattattgttgttctgtaaacatgttgcacaagcttagcctttttgcgttctgttgtgtgtggctgtaaaaccccatgctttgtgaaatgagaatcttgacatttttcttgtgaaatttggaaaatgtgatcaattgaaatcaactgtgttttgtgttctctatgtcaaagtttagttttatattgagaatgttaacttattgctttgtatcttgggaaaaaaactttgtaaataagttataaagtttctttgagacagtaaaattatgatttcttgaaaaaaaaaaaaaaaaaaaaaaa

SEQ ID NO: 3 - protein, 364 aa, Homo sapiens
MMLPSPVTSTPFSVKDILNLEQQHQHFHGAHLQADLEHHFHSAPCMLAAAEGTQFSDGGEEDEEDEGEKLSYLNSLAAADGHGDSGLCPQGYVHTVLRDSCSEPKEHEEEPEVVRDRSQKSCQLKKSLETAGDCKAAEESERPKPRSRRKPRVLFSQAQVFELERRFKQQRYLSAPEREHLASSLKLTSTQVKIWFQNRRYKCKRQRQDKSLELGAHAPPPPPRRVAVPVLVRDGKPCVTPSAQAYGAPYSVGASAYSYNSFPAYGYGNSAAAAAAAAAAAAAAAAYSSSYGCAYPAGGGGGGGGTSAATTAMQPACSAAGGGPFVNVSNLGGFGSGGSAQPLHQGTAAGAACAQGTLQGIRAW

SEQ ID NO: 4 - DNA, 2117 bp, Homo sapiens
agtccctgcagtggctgtaacaaaacccagacccccaggtcccggccaatggaggcgatttagactggagtgggaccgcgtctgtcaaaagcccgactcggcagcagcggcggagtccaggaggagagctggagccgccgcgctgcctccccgcccccgccgggatttattatttggactggacaattaagtggccctgatgatgttaccaagcccggtcacctccacccctttctcagtcaaagacattttgaatctggagcagcagcaccagcacttccatggtgcgcacttgcaggcggacttggagcaccacttccactctgcgccctgcatgctggccgccgctgaggggacgcaattttctgacggaggggaggaggacgaggaagacgagggcgagaaattgtcctatttgaactcactagccgcagcagacggccacggggattcagggctgtgtccccagggctatgtccacacggtcctgcgagactcgtgcagcgagcccaaggaacatgaagaggagcccgaggtcgtgagggaccggagccaaaaaagctgccagctgaagaagtctctagagacggccggagactgcaaggcggcggaggagagcgagaggccgaagccacgcagccgccggaagccccgggtcctcttctcgcaagcccaggtcttcgagctggaacgcaggttcaagcagcagcggtacctgtcggcacccgagcgcgagcacctcgccagcagcctgaagctcacatccactcaggtgaaaatctggttccagaatcgcaggtacaagtgcaagagacagcggcaggacaagtctctggagcttggcgcacacgcgcccccgccgccgccgcgccgcgtggctgtcccggtgctggtgcgggacggcaagccgtgcgtcacgcccagcgcgcaggcctacggcgcgccctacagcgtgggcgccagcgcctactcctacaacagcttccccgcctacggctatgggaactcggccgcggccgccgccgccgccgccgccgccgccgcagcagcggcggcctacagcagcagctatggctgtgcgtacccggcgggcggcggcggcggcggcggcgggacctccgcggcgaccactgccatgcagcccgcctgcagcgcggccggaggcggcccctttgtgaacgtgagcaacctaggaggcttcggcagcggcggcagcgcacagccgttgcaccagggtactgcagccggggccgcgtgcgctcagggcaccttgcagggcatccgggcctggtagggacggggcgggtcacgcggcgggcaccccagcgcagcctggcgccgcgggactgaagctcgagaagggcctgacctaaaggtcaggtcccctcgttaaaaaaatatgtacgtctagctcctcagggcttcggatcgcagctcactcgaggcctggggaaggggactcaggggcgaggaggatgactgggtccggtcgccaggactgtctctgaggcagaaacgccggctgggcgccggggaggacgatggccccgaccctggcagcgagaggagaccaggaggctaggaccctggccgcgcttggttcttccaaagcgagaagggcttctctccctctgcctttccgcggcctccgcgaagcgttggcggggagcccaaggacataacaaattaaaagcatgaaggagagaaaaatgggggtcgtggcttgagaaattccaggccctaccgatcctctgccccctttgcgggcctggagcgccatagcacagtcgatttcgtttcgcagctgtctcccctccgcagcagatacctcggtccagatctccggattgtcgggggacgcaggactcttcgaggaaaaccagccgaatgagatcaaaagttgggggtggggggaggctgaacaaactcaggacctggtggcccaccggaggtgttaccgggtttcctttctgtttcgtattctgtattcagcacatgttatctatctatctatctatataactataaccacacgccgtgtagacacccgctgccacacactacaggagtcaataaacaaggtgcaatattttcaaaaaaaaaaaaaaaaaaaaaaa

SEQ ID NO: 5 - protein, 258 aa, Homo sapiens
MKQILHPALETTAMTLFPVLLFLVAGLLPSFPANEDKDPAFTALLTTQTQVQREIVNKHNELRRAVSPPARNMLKMEWNKEAAANAQKWANQCNYRHSNPKDRMTSLKCGENLYMSSASSSWSQAIQSWFDEYNDFDFGVGPKTPNAVVGHYTQVVWYSSYLVGCGNAYCPNQKVLKYYYVCQYCPAGNWANRLYVPYEQGAPCASCPDNCDDGLCTNGCKYEDLYSNCKSLKLTLTCKHQLVRDSCKASCNCSNSIY

SEQ ID NO: 6 - DNA, 2219 bp, Homo sapiens
gcacaaccagaatttgccaaaacaggaaataggtgtttcatatatacggctctaaccttctctctctgcaccttccttctgtcaatagatgaaacaaatacttcatcctgctctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaa

SEQ ID NO: 7 - protein, 82 aa, Homo sapiens
MDPGPKGHCHCGGHGHPPGHCGPPPGHGPGPCGPPPHHGPGPCGPPPGHGPGPCGPPPHHGPGPCGPPPGHGPGHPPPGPHH

SEQ ID NO: 8 - DNA, 464 bp, Homo sapiens
agacttcctgcccctgctctgcactctcaggtattccctgctcttactccaaaaagatggacccaggtccgaaggggcactgccactgtggggggcatggccatcctccaggtcactgcgggccaccccctggccatggcccagggccctgcgggccacccccccaccatggtccagggccctgcgggccaccccctggccatggcccagggccctgcgggccacccccccaccatggtccagggccctgcgggcctccccctggccatggcccaggtcacccaccccctggtccacatcactgaggaagtagaagaaaacaggacacaagatggcaagcctgagagaattgcccagctgacctggaatgaggcctaaaccacaatcttctcttcctaataaacagcctcctagaggccacattctattctttaaaaaaaaaaaaaaaaaaaaaaaaaaaaaaa

SEQ ID NO: 9 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 10 - DNA, 3233 bp, Homo sapiens
cagcagccagctccccagcaccgcacggcggggacgcgagcgcgcccccgacggcagcccggacgccgagcacgggtcacctgcggcgccggcccgggcgccgaccgaggttcaacgcacggcccggggacccccaggcggggccaacgccgccgtcgcccccggcctcgcggggagcaggaagagccaacatgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaaaaaaaaaaa

SEQ ID NO: 11 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGLKSDELPNQMSMDDWPEMKKKFADVFAEKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 12 - DNA, 3352 bp, Homo sapiens
ggggcgtggcgccggggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaa

SEQ ID NO: 13 - protein, 238 aa, Homo sapiens
MLCCMRRTKQVEKNDDDQKIEQDGIKPEDKAHKAATKIQASFRGHITRKKLKGEKKDDVQAAEAEANKKDEAPVADGVEKKGEGTTTAEAAPATGSKPDEPGKAGETPSEEKKGEGDAATEQAAPQAPASSEEKAGSAETESATKASTDNSPSSKAEDAPAKEEPKQADVPAAVTAAAATTPAAEDAAAKATAQPPTETGESSQAEENIEAVDETKPKESARQDEGKEEEPEADQEHA

SEQ ID NO: 14 - DNA, 3710 bp, Homo sapiens
tctctgggttgttttcaacatctcaagtgtgaattttccctgtcaaaatcttcacaaggaaaatgagtcacagcatcacctgggtgacgaggtcataacacctcagcccttgcttaaaaaattttatttctacttttctattgtaaagagatctcaaaacaggaagataaaattggactgacagctctacagcctagtcttttagacagtgaactaggccagcattggcagacactggcgatgacaaagtcctgctctgaattatgccaccccgcactccactttttaccttgcctgggaggcttgaggaaaaatcttcagagagcagttcgacctagtccttattcacttggcttcttgactttctggatttcaagggtaattttacctcacatgtaacctatacaagtattttaggttaaaaccctgactctgccacttactagctgtatgactttgagcaaattacttaattacttaactaatgtccttctacttagtcttttcatctgtaagataatagtacccattgcatagttttgttcttagaattgaattagttaatatacaaactatatgcaataaatatgtttggggtgtgtataatcatataaaagtgcttagaagagtgcttgacatatagttttagaatttgctattattatgttctccaagcatatgagggtattttatgtcattacttttaaaggtcgcttttttatatgtgttagtgggaggcatttattatttttttaagtgtgcatatccctgaaacatccctctgtggagctataaaagaaaactataaaatatttaccatataattcactcaatccccagtctccagaaagtaacaatttgtacttcatttgtattgtcttcttccaggcattttacaaatactattttctttcatcctcaaaaaaactctgcacaataggtaataggattctaatatgggtgataagaaaactgaaatttagagaaattaagtaatttatcccaggtcatacatttaagcagtgaatgagccaggattcaaactcagatcttttgaccccaaaaccatgccaggtagcaagtaatcaatgaatgtcaatgacagatcatcgagagagaatccagtgccaaaaatgaagagggtgtcaacaaaataagaacaaggacaatggcagcatgagcaacaagaacaaacttagatccaaaattaacagttaaagatttttctagagtcctgcatatttttccttaaactgtccagcagcttggggacagaaatctaattaaaatggaagtctatgcatgctcaaaccacatgccttgtacctgatgcaatccgcttccaccccagtcctgaaaaaaggcattcagttatttttttctacatgtggaagaatacaacaacataccttctagaatcagggagaaatgtacttgctctttttatttttttttaatttctcaaatactgttctataaggataatcactcacctggtgattaatgtagaatgttaaggtgtgtacaaattactgctgataggtaacactgtccttgcaaaatcatcccatgtgcccccctcaaataaataagtatacttcaaaaagcagtgtgcttgctatacaagtgcagttttaagaaaaatcactcagctctctcttgcaaacatgttctttcccctaatgcctcctacacagagattctggagccacccccttcagaatatctgaaatatagaccaattttgctcgttaaaactacttcaagtagtcctagagctgatattaggattgctacttattcagtcatacttttcgagtgttcattgttgaattcttagattgaattttctattagtttccttaaaatataagaggttggagggctaagtttaagacccattctactaattgtgaggctttccacaatctgtgtttccttatttttttaaaggggatggcacacttgcttagccttcttcattatcattctcagaggaatcattttaagttgaaagtacttcataacctataatgcataaatttaaaccactgtcatttccccccaccacagcccttggcaaatggggtcttaaattccttctcccaccaactagacttttagcatgcattctgacacgtattgtgtatccttactagatatgaggtatataatatttgggctttactaactatccagtgggggtgcattggatatacttggcttggcaatttcaaattataatgatcttcagtaccagatgctcttaagctagtcccctacaaatgtctggcagcttcatttgtaacttttatcaggcttgagtattaccacacacaagcacatagagtagctctcagaacctgcagaggcaattgttttactagatgtgtgagggatatagttggatcaatgggatagatactgtatatgaaatagtggctaaatgagtttgtgtgtgtgtgtgtgtgtgtgtgtgtgtgtggttgggggatgtcaggtgaaggtcacatattttgggaatgaatagatttctagtttcagtgaatttctatcactaagctcctacactaggtgaagttttggggggtgggtaagttagagaagactgagaaggaatcagttggtgtgtgtcaggtgatagtagagtgggactgtggtttaactttgacaatgtaaacagatcttcccccttcccaagataagaaagaacagagttgagtactatggcacattttacatggtttcctgcttatctctttctttgagaatagatatagctcagtagtactgtcgagttaagaaatagcttttccaccctctgagtgatgatacctacaatcatcaggatgaatacagtctcatgaccaaatctcctctggtatttgtcttatcaacatgccttaacttaatgtcacgatgctctagaaattactctgagagaataatggtgatggcaataattatgatgagaataatggtagcagccacccctttctgagtatgtacaaagtgtcagggactgtgctgggtgctttatgtgcattaactcatttgattctcatatcagcccaatgggatagtttttttattccctcatcctgcagatgaggagcctaaagctgacttgcccaaggtcaggatctaccaagtaactggcagagctggaattcaaacttacacatgtctaactctgcagcccatgctcttaagcaccagggaatattgacttttacaggaggaggactaagcaagggctttgtcattcagaagctggatgacgctgggctccttcctggttcctcatctgtgttgattataaaacaggcacagtgatgtgttcctcacatgatcatgatgaggattgaattgaaatactaaatatatatactgtgcacttataaatgtaagaagagagaggaaaatgactgctggagagtggcgtgatgaaggctccagcattagtgacctggagcgagaacacccagccagtgagctgagtgttcacacacagagagtttacacactctctgagcaccaacacagtatggcatgtggtcatgcaacaactaaaaataacatttttttttagtatcaactatgtgccagcactattttagccactgaagcttagaagattgtaaaaaaacagataaagttctgttctcagattgtttatagcccaagcgaagttcagttagtaataaaat

SEQ ID NO: 15 - protein, 330 aa, Homo sapiens
MLPDWKSSLILMAYIIIFLTGLPANLLALRAFVGRIRQPQPAPVHILLLSLTLADLLLLLLLPFKIIEAASNFRWYLPKVVCALTSFGFYSSIYCSTWLLAGISIERYLGVAFPVQYKLSRRPLYGVIAALVAWVMSFGHCTIVIIVQYLNTTEQVRSGNEITCYENFTDNQLDVVLPVRLELCLVLFFIPMAVTIFCYWRFVWIMLSQPLVGAQRRRRAVGLAVVTLLNFLVCFGPYNVSHLVGYHQRKSPWWRSIAVVFSSLNASLDPLLFYFSSSVVRRAFGRGLQVLRNQGSSLLGRRGKDTAEGTNEDRGVGQGEGMPSSDFTTE

SEQ ID NO: 16 - DNA, 2069 bp, Homo sapiens
atgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccttgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtagcagtttccctggaccttcagaggtcgcctgggttacacaggagctgggaagcctgggagaggcggagcaggaaggctcccatccagattcagaaatccttagacccagcccaggactgcgactttgaaaaaaatgcctttcaccagcttggtatcccttcctgactgaattgtcctactcaaaggagcataagtcagagatgcacgaagaagtagttaggtatagaagcacctgccgggtgtggtggctcatgcctataatcccagaactttgggaggctgaggcaggtggatcacttgaggtcgggagattgagaacatcctggtcaacatgggaaaaccccgtctctactaaaaatacaaaaaaattagctgggcatggtggcacatgcctataatcccagctactctggaggctgaggcaggagaatccttgaacccgggagttggaggttgcagtgagctgagatcacgccactgcactccagcctagcgacagagcaagactccatttaaaaaaaaaaaaaaaaaaaaaaagaagcaccttcaggctggagaagcagcgtagctaacacaagtccagtccttgtgatgtggctggtagttggggatggccaggctgaagcagagagtcctagagaaatctcgatacaagcttcaaagcaacacctagacactgctctagcggttgatcctggagataaaccaacaagagagagatggaagagaaatactaaatgaggtcaaagaagactcagaaaggttctgagcctggagatgagcagggaggcctcagggcttagacctttaatgataggggtttccctgcattggtttgacctgttgcctttttgatgtgctctgtttgttttcatgtgttgtcttgtctcccctgctaaactgggagctgccaggggtctgggtcttatctccttcctccatggtaccccacacaggccaggatgtggtttggtacccagcaatcagagattggcactccctcatacaggggaaagcaacctggtctagcaaattgaaaataaagatgataaaactctgaaaaaaaaaaaaaaaaaa

SEQ ID NO: 17 - protein, 428 aa, Homo sapiens
MLRTLLRRRLFSYPTKYYFMVLVLSLITFSVLRIHQKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 18 - DNA, 5984 bp, Homo sapiens
acagaccataggtgaactgttaaggagggagaagccgcagtgtcaccttccacagtcctgctcccattaagacctatgaagtggaaaatgcagattattctttgaggtttcctgggaccagaaaaggtcagagactcttaatgggtaatgcagacaactggaatgcaatcatcaggtattcatttctgttttaaggttttatgtctgagaaacctgaaacctgaagaagtaaagcaatcatgattttttaaaaacttaataatatggttttggatttaaaaagaaagactgactcagagctggggaaaacactcacttgtgatacggatcaatgtgaaaacagcttaactctgttccatggcaattcgcacagtgtgactcagagacacctctcttcaccagctctggctcaggggatgtgaagccagaagcagaggtcacagtgacttcatctgagaaggacttaactgaccattgctggttttgaagccggaggaaggcccacagtcaatgaatgagagaggcttcgagaagctagaaatggcaaggaaatagattttcctctaaagctcccagaaagaaacacggttctgctgacacctcgattttaggccagtgagacctgcttcagacttctgacctacagaacgatttctttaaagactcgtgcagcacatcattatcgctggatgcccggacatgtaatacacctgacagcatgtgaagtgctcagaatggggcaggatgtcacctggaatcagcactaagtgattcagactttccttacttttaaatgtgctgctcttcatttcaagatgccgttgcagctctgataaatgcaaactgacaaccttcaaggccacgacggagggaaaatcattggtgcttggagcatagaagactgcccttcacaaaggaaatccctgattattgtttgaaatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactgaccattacgggcaattttatgaacaagaagaaggatacacaaaacgtacccttatctgtttccccttccttgtcagcatcgggaagatggtatgaagtcctctttggggcagggactctagtagatcttcttgtcagagaagctgcatggtttctgcagagcacagttagctagaaaggtgatagcattaaatgttcatctagagttaatagtgggaggagtaaaggtagccttgaggccagagcaggtagcaaggcattgtggaaagaggggaccagggtggctggggaagaggccgatgcataaagtcagcctgttcaaagtgctcagggacttagcaaaatgagaagatgtgacctgtgccaaaactattttgagaattttaaatgtgaccatttttctggtatgaataaacttacagcaacaaataatcaaagatacaattaatctgatattatatttgttgaaatagaaatttgattgtactataaatgatttttgtaaataatttatattctgctctaatactgtactgtgtagtgtgtctccgtatgtcatctcagggagcttaaaatgggcttgatttaacattgtttttgtgttatttttgcttgaaacaacgcacacattttcaacaaccaaaaaatgacaatttctagtttagttaatttctacaaatcatcttatgttattagcaaggttaagacatcttttttaaaaaaattatagcttctaccaagagaaacactcaatttttctagagatttgcctctatcttcctttcctcagtcttcccagactgctatcaagctgtgtaaaaatttactttcactggaccctaaattattgtctctgctatctgactgccagtaattagtgcagaaaactaagacaggatgatacaggtttgaggggctggggagtgggaggggggagaaaaggaatgtatttaaacaatttccgatgcccatgatgagtttaaaaaccagcattgacaccatccccaaaattaaggctgtcgcttattgaatccacttgtgtccaacctcccaggattgttttatcctaatgtcacctgtatattcatttgaaaggacttggccctgttcttgggtcttcccgttacctgccccctgggtggtaagtttcctcctttctcaaccttccacgaggaggaaagaagtgtgcagtcattccacatggcctgttggaaggcctggggagggaactttgggtttgggacagatttttttttttgtttttggtatcattcacagcatacgatttttactctctccatcttcaccataagacagataatttggggttgctataatgctgtcacacatctcaaagtacattcaaatcttaaaaagaaattctcgtacttttgccatgttgatactgttcagcaaacaagctaccaggaactgtgaggctttgtcatttagcattagactttaaacaagaattaaaatcatgtgctgtatttttaaaatctagccaaattaaatagtacatgagaaattcagagtattagacagttttaaggcattcaactgagaaaactttatttgtcaaagtcagaaaacattttcatcttattgagagatatgtttttaaacttttatcatcatttgtaaatgtggaagttggtggattgctgtgtttttgcatgattagcatgggagtctgttggagcaagaggaacatgcttgttttgaaaactcgagatgatgagggtggtacatgcagtgtgttctctctttattggcttctaaaccagttttgtcctttaatgcatgtcaaatatttctcccatgcttctcttagcagaaaagtttttacctataagacagggcaccttttaactctaaaactagtgatactcagtgacatagactttgtcttataaacattttttcattttttattttgaaaaattgcaaatctacagcaaaagtaaaacagtagagtgaacaccatgtaaccctcacctggtgttaacattgtaccctatttgctttaagttgtatgtatttctgaacttggcaaaattggaaattaaaatttttaaaaattacaaatatacaaagttatttatcttagcacatttattatgtgtgactgcatctgatttatatttaaattggcaggttttgagggatttttttcttcatcataaatgtaaacataggattttagagtctatttccccaagcgccacattataactgtaaacttaccatcttctatgtagctgtgatatctcatctttctaaaatggaacttgttaaaaagtgttcaaacactatccctaatgcctgcggcagaatttatatacgatccattcattggggctcaaagtatcttttagacttttaaggacaatttacagcaaatgaaatttatgatgctgtgacaagaaatttaaagaatcaaaacgatggtttgaaaaggaaacctatgatacatgcaggagaagcaaaacccaagtgattggtgagaaatatagaaattatttaaattactttatagtaattattaaacactaatttttgtactgtcattgaaggtgttttatagagaaatctgagaaatcacattcacaaattagaagtcaaacatggccaggcacagtggctcacacctgtaatcttcaggatttcaagaccagcctagccaacatgacgaaaccccatctctactgaaaatagaaaaaaaaaattagccaggcttgcacctgtaatcccagttacctgggaggctgaggcaggagaatctcttgagcccaggaggcggaggttgcagtgagctgagatgccaccaccacaccagcctgggtgacagagtgaaactcgtatctccaaacaaacaaacaaaaagtccttaaacatatgtgaacaaaaattttgtgatggaaggattctagttaatgagtattgcatcaagatttacatctttcttactaaggaaaagagttaataaaaattgttctttattttacaggcagttactgaggctcttcccagatctcagtaaacagccactcagccttgaaaatggagtgttgttgtttctaaacatatatttatgtcatttattaagtacagttcacttaaataacataagtagattttctcttgtagtgatttgggtaggaagaggccatgtttcagttcgttttctctgtagggtcgattgaattggaccttttcagttgttcagaaaaataaaaataatttctcatattaaatacagacgctcctcaacttatgatgtgggtaggtcccagtaaacccattataatttgaaaatatcacattgaaaatgcatttaatatctcttacctgaaatcataacttagccaagcctaccttaaatgttctcagaacatttagcctgcagttgggcaaaaccatttaacacaaagcctattttataatgaagtgttgaatagctcatgttatttactgaatactgttgtgaaagtgaaaaacaatgattgtatgggtactcaaagtataatttctactgaatgcatatcacttgtgcactgttgtaaagctgaaaaaccgttaagcctctacgatttttaagtaagttggggaccatcagtttaaaataaatgcaatactatttcatgataaacatggtcactgtaagttttactcttttgaatgagggtgcgacagaatgcagttagaatcagttcatatcaccattaaaatcatccattcagaaaccaaaaaaaaaaaa

SEQ ID NO: 19 - protein, 667 aa, Homo sapiens
MKEKSKNAAKTRREKENGEFYELAKLLPLPSAITSQLDKASIIRLTTSYLKMRAVFPEGLGDAWGQPSRAGPLDGVAKELGSHLLQTLDGFVFVVASDGKIMYISETASVHLGLSQVELTGNSIYEYIHPSDHDEMTAVLTAHQPLHHHLLQEYEIERSFFLRMKCVLAKRNAGLTCSGYKVIHCSGYLKIRQYMLDMSLYDSCYQIVGLVAVGQSLPPSAITEIKLYSNMFMFRASLDLKLIFLDSRVTEVTGYEPQDLIEKTLYHHVHGCDVFHLRYAHHLLLVKGQVTTKYYRLLSKRGGWVWVQSYATVVHNSRSSRPHCIVSVNYVLTEIEYKELQLSLEQVSTAKSQDSWRTALSTSQETRKLVKPKNTKMKTKLRTNPYPPQQYSSFQMDKLECGQLGNWRASPPASAAAPPELQPHSESSDLLYTPSYSLPFSYHYGHFPLDSHVFSSKKPMLPAKFGQPQGSPCEVARFFLSTLPASGECQWHYANPLVPSSSSPAKNPPEPPANTARHSLVPSYEAPAAAVRRFGEDTAPPSFPSCGHYREEPALGPAKAARQAARDGARLALARAAPECCAPPTPEAPGAPAQLPFVLLNYHRVLARRGPLGGAAPAASGLACAPGGPEAATGALRLRHPSPAATSPPGAPLPHYLGASVIITNGR

SEQ ID NO: 20 - DNA, 4177 bp, Homo sapiens
ggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtttaggagacgcgtggggacagccgagccgcgccgggcccctggacggcgtcgccaaggagctgggatcgcacttgctgcagactttggatggatttgtttttgtggtagcatctgatggcaaaatcatgtatatatccgagaccgcttctgtccatttaggcttatcccaggtggagctcacgggcaacagtatttatgaatacatccatccttctgaccacgatgagatgaccgctgtcctcacggcccaccagccgctgcaccaccacctgctccaagagtatgagatagagaggtcgttctttcttcgaatgaaatgtgtcttggcgaaaaggaacgcgggcctgacctgcagcggatacaaggtcatccactgcagtggctacttgaagatcaggcagtatatgctggacatgtccctgtacgactcctgctaccagattgtggggctggtggccgtgggccagtcgctgccacccagtgccatcaccgagatcaagctgtacagtaacatgttcatgttcagggccagccttgacctgaagctgatattcctggattccagggtgaccgaggtgacggggtacgagccgcaggacctgatcgagaagaccctataccatcacgtgcacggctgcgacgtgttccacctccgctacgcacaccacctcctgttggtgaagggccaggtcaccaccaagtactaccggctgctgtccaagcggggcggctgggtgtgggtgcagagctacgccaccgtggtgcacaacagccgctcgtcccggccccactgcatcgtgagtgtcaattatgtactcacggagattgaatacaaggaacttcagctgtccctggagcaggtgtccactgccaagtcccaggactcctggaggaccgccttgtctacctcacaagaaactaggaaattagtgaaacccaaaaataccaagatgaagacaaagctgagaacaaacccttaccccccacagcaatacagctcgttccaaatggacaaactggaatgcggccagctcggaaactggagagccagtccccctgcaagcgctgctgctcctccagaactgcagccccactcagaaagcagtgaccttctgtacacgccatcctacagcctgcccttctcctaccattacggacacttccctctggactctcacgtcttcagcagcaaaaagccaatgttgccggccaagttcgggcagccccaaggatccccttgtgaggtggcacgctttttcctgagcacactgccagccagcggtgaatgccagtggcattatgccaaccccctagtgcctagcagctcgtctccagctaaaaatcctccagagccaccggcgaacactgctaggcacagcctggtgccaagctacgaagcgcccgccgccgccgtgcgcaggttcggcgaggacaccgcgcccccgagcttcccgagctgcggccactaccgcgaggagcccgcgctgggcccggccaaagccgcccgccaggccgcccgggacggggcgcggctggcgctggcccgcgcggcacccgagtgctgcgcgcccccgacccccgaggccccgggcgcgccggcgcagctgcccttcgtgctgctcaactaccaccgcgtgctggcccggcgcggaccgctggggggcgccgcacccgccgcctccggcctggcctgcgctcccggcggccccgaggcggcgaccggcgcgctgcggctccggcacccgagccccgccgccacctccccgcccggcgcgcccctgccgcactacctgggcgcctcggtcatcatcaccaacgggaggtgacccgctggccgcccgcgccaggagcctggacccggcctcccggggctgcggcgccaccgagcccggcaaatgcgcacgacctacattaatttatgcagagacagctgtttgaattggaccccgccgccgacttgcggatttccaccgcggaggccccgcgcgccggtgccgagggccgaggagcgcccgggtccgggcaggtgaccgcccgcctctgtcctgcgagggccggtgcgacccagttgctgggggcttggtttcctcaccttgaaatcgggcttcacgcgtcttgccttgtccccaacgttccacaacagtcccgctgggggattgaagcggtttcactccgcaaatatcctccactttcaggagggaaaacccaccctaccacagtccgctcttccaagtggacggcagacctgggaggggacgcctgtgtcacgagcccttttagatgcttaggtgaaggcagaagtgatgattgtaagtcccatgaatacacaactccactgtctttaaaagtcattcaagagtctcattatttttgtttttatttaaccctttcttcaatacaaaaagccaacaaaccaagactaagggggtgaccatgcaattccattttgtgtctgtgaacataggtgtgcttcccaaatacattaacaagctcttacttccccctaacccctatgaactcttgataacaccaagagtagcaccttcagaatatattgaataggcattaaatgcaaaaatatatatgtagccagacagtttatgagaatgaccctgtcaagcttcattattacgtggcaaaatccctctggcccacacagatctgtaattcactaggctcgtgtttgctacaaatagtgctaataaagttaaattgcacgtgcaatacggaacactgtcaatggactgcaccttgtgaaggaaaaacatgcttaagggggtgtaatgaaaatgatgtagacattttaagcattttctacacagcgagaaaacttcgtaagaacatgttacgtgtgcaacaggtaaacagaaatcctttcataaagcaccagcagtgtttaaaaaatgagcttccattaatttttactttttatgggttttgcttaaagatctcaacatggaaaaatcctgtcatggctctgaactgcacaatgcattgaaccgccgtccttcaattttcttcacactatcaacactgcagcattttgctgctttatcaaaatggtttattttaggaaactttttccacctttctgaatggaaagaggttttcacaaatgttttaaactcatcgttctaaaatcaagtgcacctacaccaactgctctcaaaatgtgaactgacttttttttttttttttttgccaaccctgtgtcacttagtgaggacctgacacaatccctacagggtgtctgtcagtgggcctcatggtaagagtcacaatttgcaaatttaggaccgtgggtcatgcagcgaaggggctggatggtaggaagggatgtgcccgcctctccacgcactcagctatacctcattcacagctccttgtgagtgtgtgcacaggaaataagccgagggtattatttttttatgttcatgagtcttgtaattaaaccgtgattcttgaaaggtgtaggtttgattactaggagataccaccgacatttttcaataaagtactgcaaaatgcttttgtgtctaccttgttattaacttttggggctgtatttagtaaaaataaatcaaggctatcggagcagttcaataacaaaggttactgttgagaaaaaagaccctatcatagatttacaagtgacaacgtggacaggcgtaacagagtgtgtatgtacttgaaaatgtgtactagtgaaaacgtccttgtctaaacaaacttcgttttcataacctagaacatgaaggaagattcttacacgagctggcttcaggacggccagggctgagtgtgaagtcagcagagctgttactgtggcaacctaatattgtgacctccatcttgaagagtctgcacatttaagctggggatagcgtttcttgacttctgtgaggctccggcaggggaagaggaagcattttaatacatatgc

SEQ ID NO: 21 - protein, 294 aa, Homo sapiens
MKDRLQELKQRTKEIELSRDSHVSTTETEEQGVFLQQAVIYEREPVAERHLHEIQKLQESINNLADNVQKFGQQQKSLVASMRRFSLLKRESTITKEIKIQAEYINRSLNDLVKEVKKSEVENGPSSVVTRILKSQHAAMFRHFQQIMFIYNDTIAAKQEKCKTFILRQLEVAGKEMSEEDVNDMLHQGKWEVFNESLLTEINITKAQLSEIEQRHKELVNLENQIKDLRDLFIQISLLVEEQGESINNIEMTVNSTKEYVNNTKEKFGLAVKYKKRNPCRVLCCWCCPCCSSK

SEQ ID NO: 22 - DNA, 1198 bp, Homo sapiens
gtgtgaagtaagcaatctattttcacttgctattaacagcaaacgtacaactttatctcttatttgacttcattatttggctttcaaattaacaacatgaaaactatacagaaattagtctgtgccctataagacagagatttagataatttgttcaaaacatgagatgatattcagatattctctcaaaggcaggactccaacaccgcaggtggaaaaccaacaaaaggaagaaaacagaaaggaggaaagggaagatgaaagaccgacttcaagaactaaagcagagaacaaaggaaattgaactctctagagacagtcatgtatcaactacagaaacagaggaacaaggggtgtttctacagcaagctgttatttatgaaagagagcctgtagctgagagacacctacatgaaatccaaaaactacaggaaagtattaacaatttggcagataatgttcaaaaatttgggcagcaacagaaaagtctggtggcttcaatgagaaggtttagtctacttaagagagagtctaccattacaaaggagataaaaattcaggcagaatacatcaacagaagtttgaatgatttagttaaagaagttaaaaagtcagaggttgaaaatggtccatcttcagtggtcacaaggatacttaaatctcagcatgctgcaatgttccgccattttcagcaaatcatgtttatatacaatgacacaatagcagcaaagcaagagaagtgcaagacatttattttacgtcagcttgaagttgctggaaaagagatgtctgaagaagatgtaaatgatatgcttcatcaaggaaaatgggaagtttttaatgaaagcttacttacagaaatcaatatcactaaagcacaactttcagagattgaacagagacacaaggaacttgttaatttggagaaccaaataaaggatttaagggatcttttcattcagatatctcttttagtagaggaacaaggagagagcatcaacaatattgaaatgacagtgaatagtacaaaagagtatgttaacaatactaaagagaaatttggactagctgtaaaatacaaaaaaagaaatccttgcagagtactgtgttgttggtgctgtccatgctgtagctcaaaataaagaagctattttaaaaaaaaaaaaaaaaaaaaaaaaaaaaaaaaaaaaaaaaaaaa

SEQ ID NO: 23 - protein, 1044 aa, Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 24 - DNA, 6079 bp, Homo sapiens
atcctcagtctcccagttcaagctaatcattgacagagctttacaatcacaagcttttactgaagctttgataagacagtccagcagttggtggcaaatgaagccaggctgtgcggcaggatctccagggaatgaatggattttcttcagcactgatgaaataaccacacgctataggaatacaatgtccaacgggggattgcaaagatctgtcatcctgtcagcacttattctgctacgagctgttactggattctctggagatggaagagctatatggtctaaaaatcctaattttactccggtaaatgaaagtcagctgtttctctatgacactttccctaaaaactttttctggggtattgggactggagcattgcaagtggaagggagttggaagaaggatggaaaaggaccttctatatgggatcatttcatccacacacaccttaaaaatgtcagcagcacgaatggttccagtgacagttatatttttctggaaaaagacttatcagccctggattttataggagtttctttttatcaattttcaatttcctggccaaggcttttccccgatggaatagtaacagttgccaacgcaaaaggtctgcagtactacagtactcttctggacgctctagtgcttagaaacattgaacctatagttactttataccactgggatttgcctttggcactacaagaaaaatatggggggtggaaaaatgataccataatagatatcttcaatgactatgccacatactgtttccagatgtttggggaccgtgtcaaatattggattacaattcacaacccatatctagtggcttggcatgggtatgggacaggtatgcatgcccctggagagaagggaaatttagcagctgtctacactgtgggacacaacttgatcaaggctcactcgaaagtttggcataactacaacacacatttccgcccacatcagaagggttggttatcgatcacgttgggatctcattggatcgagccaaaccggtcggaaaacacgatggatatattcaaatgtcaacaatccatggtttctgtgcttggatggtttgccaaccctatccatggggatggcgactatccagaggggatgagaaagaagttgttctccgttctacccattttctctgaagcagagaagcatgagatgagaggcacagctgatttctttgccttttcttttggacccaacaacttcaagcccctaaacaccatggctaaaatgggacaaaatgtttcacttaatttaagagaagcgctgaactggattaaactggaatacaacaaccctcgaatcttgattgctgagaatggctggttcacagacagtcgtgtgaaaacagaagacaccacggccatctacatgatgaagaatttcctcagccaggtgcttcaagcaataaggttagatgaaatacgagtgtttggttatactgcctggtctctcctggatggctttgaatggcaggatgcttacaccatccgccgaggattattttatgtggattttaacagtaaacagaaagagcggaaacctaagtcttcagcacactactacaaacagatcatacgagaaaatggtttttctttaaaagagtccacgccagatgtgcagggccagtttccctgtgacttctcctggggtgtcactgaatctgttcttaagcccgagtctgtggcttcgtccccacagttcagcgatcctcatctgtacgtgtggaacgccactggcaacagactgttgcaccgagtggaaggggtgaggctgaaaacacgacccgctcaatgcacagattttgtaaacatcaaaaaacaacttgagatgttggcaagaatgaaagtcacccactaccggtttgctctggattgggcctcggtccttcccactggcaacctgtccgcggtgaaccgacaggccctgaggtactacaggtgcgtggtcagtgaggggctgaagcttggcatctccgcgatggtcaccctgtattatccgacccacgcccacctaggcctccccgagcctctgttgcatgccgacgggtggctgaacccatcgacggccgaggccttccaggcctacgctgggctgtgcttccaggagctgggggacctggtgaagctctggatcaccatcaacgagcctaaccggctaagtgacatctacaaccgctctggcaacgacacctacggggcggcgcacaacctgctggtggcccacgccctggcctggcgcctctacgaccggcagttcaggccctcacagcgcggggccgtgtcgctgtcgctgcacgcggactgggcggaacccgccaacccctatgctgactcgcactggagggcggccgagcgcttcctgcagttcgagatcgcctggttcgccgagccgctcttcaagaccggggactaccccgcggccatgagggaatacattgcctccaagcaccgacgggggctttccagctcggccctgccgcgcctcaccgaggccgaaaggaggctgctcaagggcacggtcgacttctgcgcgctcaaccacttcaccactaggttcgtgatgcacgagcagctggccggcagccgctacgactcggacagggacatccagtttctgcaggacatcacccgcctgagctcccccacgcgcctggctgtgattccctggggggtgcgcaagctgctgcggtgggtccggaggaactacggcgacatggacatttacatcaccgccagtggcatcgacgaccaggctctggaggatgaccggctccggaagtactacctagggaagtaccttcaggaggtgctgaaagcatacctgattgataaagtcagaatcaaaggctattatgcattcaaactggctgaagagaaatctaaacccagatttggattcttcacatctgattttaaagctaaatcctcaatacaattttacaacaaagtgatcagcagcaggggcttcccttttgagaacagtagttctagatgcagtcagacccaagaaaatacagagtgcactgtctgcttattccttgtgcagaagaaaccactgatattcctgggttgttgcttcttctccaccctggttctactcttatcaattgccatttttcaaaggcagaagagaagaaagttttggaaagcaaaaaacttacaacacataccattaaagaaaggcaagagagttgttagctaaactgatctgtctgcatgatagacagtttaaaaattcatcccagttccatatgctggtaacttacaggagatatacctgtattatagaaagacaatctgagatacagctgtaaccaaggtgatgacaattgtctctgctgtgtggttcaaagaacattcccttaggtgttgacatcagtgaactcagttcttggatgtaaacataaaggcttcatcctgacagtaagctatgaggattacatgctacattgcttcttaaagtttcatcaactgtattccatcattctgctttagctttcatctctaccaatagctacttgtggtacaataaattatttttaagaagtaaaactctggggctggacgctgtggctcacacctgtaatctcagcactttgggaggccgaggcggggagatcacctgaggtgaggagttcgagaccagcctggccaacatggtgaaaccatgtctctactaaaaatacaaaaaattagccaggcgtggtgacagtggcacctgtaatcccagctacttgggaggctgaggcagaagtttgaacccaggaaacaggttacagtaggccaaaattgcgccactgcactccagcctaggcgacaacagcaagactgtgtccaaaaaaaaaaaaaaaagcaaaagcaaaactttgttttgttagactctacagcagagatttaacacccttctttaaactgggtagtcagtgatagataatatatattctgtcacttctaataaggtgccttctcctttaggtcagggtggttctaaaatggaaagaaaacacaatagggtaagtagtgcttgtctaagccagttacaacacagactcttaaagaggatcaagcccttcatttttctaacaacaaaaaatcacctatagaatatctaatttgtgatcttttactagatctgattttttaaaataatgtaatttccggccaggcacggtggcaccgcctgtaatcccagcactttgggaggccaaggcaggtggatcacctgaggttaggagttcgagactagcctggccaacatggcaaaaccccatctctactaaaaatacaaaagttagccgggcatggtggtgggcacctgtaatcccagctactcaggaggccgaggcaggagaatcgcttgaacccgagaggcagaggttgcaatgagccaagatcgtgccattgcactccagcctgggggacagggcaagactgtctctcaaaataaaaaaaaataataaaaataaaaataaaagtaatttccaaaacctcatctcatggaaagatcacaggatgaaggaaagctagactcaactctgtgaatagaagttgctatactgtaagtaaagcaacaattcagaatactgaatgagtttaaattgttttatatagcacccttttgggctagggttaattactagatctgacttggataatttgacactttgggaaatgaactctgttcttgagacttgttcagtgtattttaaacatctgaggaagaaaacttaaatatgcacctatttatacctattctttctttaggtcaacatttaacacccactgcatacattaatttgtccttgtctgctcactccagcaatttagaccttaacagtcacaagagacgttcttctgttacaaagccttagtaaattaaggcagttttgattatattctaggtccacctatgtctgaagctaaattcagtatctaactgctaatgaacaagtttccaaaatactgtaaaaatacaattagtcaatttgagtaaatgcaaatatgatgagaaatcaatttgctatttggcctggcaaatgggaacagtaaaattctgctttactcttctctagtctccttgccccagctgcacccactaccccaaagttggcagttttgaggtatgattttcaaggaatttttttagtattaacatctccctctgagaactatgtacctaaggtcacgcatacaactagtcaattctgtttttattactctaactatgtagaaacagtaagtcacttaaaacaatcacttggctgggttttttcccctttgtgccacattgattcaccctgacccaagaactccagggaaaattctttaatgtcaactgggcaactcattaacctctctttaacatcaagggcttgggaaaaaaaaaaaaaaggttagccacaggaataacaaaaacctggaatttatctttcaggttttgctttctctttctcactttgtttaaagtatctcgtactcacagttcacaaattaaccttcactgtctctttcacattaagagcttatgcttaaagcatgccccccttttctaacttgctggtttaccataaactcccctaagtaataaaattcctaacccagtactgagagtcctccttctctgccacttgggcattattttactagtttttaagccatcatcgcacaagaatccaaaaacccttaaattttttaaccactggcaaatatgtacagcaaattaggttaagcatttaatctggctcatgctctatcatactaaatattcaggtttatcataaactccttaaaaaccatcaaaggtcaaccagaaactgataactcttgaaaggagcaaacaggtaagatctttggagtttaagcttttctgagatgtgttgtgaaaaatctaacgtgtttatcgtatattcaatgtaacaacctggagaatcacaactatatttaaagagcctctggaaaatgaggccagtacagtgtgactacatgtttaattttcaatgtaatttattccaaataaactggttcatgctgaccacttgtattcaactaa

SEQ ID NO: 25 - protein, 326 aa, Homo sapiens
MTGLCGYSAPDMRGLRLIMIPVELLLCYLLLHPVDATSYGKQTNVLMHFPLSLESQTPSSDPLSCQFLHPKSLPGFSHMAPLPKFLVSLALRNALEEAGCQADVWALQLQLYRQGGVNATQVLIQHLRGLQKGRSTERNVSVEALASALQLLAREQQSTGRVGRSLPTEDCENEKEQAVHNVVQLLPGVGTFYNLGTALYYATQNCLGKARERGRDGAIDLGYDLLMTMAGMSGGPMGLAISAALKPALRSGVQQLIQYYQDQKDANISQPETTKEGLRAISDVSDLEETTTLASFISEVVSSAPYWGWAIIKSYDLDPGAGSLEI

SEQ ID NO: 26 - DNA, 1722 bp, Homo sapiens
aggcagaacaggatcaggaagcgatcaaacctaccaaggcagtctcacttctcaatgactggactgtgtgggtactctgctccagacatgcgtggcctcagactcatcatgataccagttgagctgctactttgctacctcctgctgcaccctgtggatgccacttcatatggaaagcagacaaatgtcttgatgcactttcccttgtccttggaatcccagacaccctcctcagaccccttgtcctgccaatttctgcacccaaagtcactgcctggtttcagccacatggcccctctacccaagttcttggtaagcctggctctaaggaatgccctggaggaagctggttgtcaggctgatgtttgggctctacagctacagctctaccgccagggtggtgtgaatgctacacaggtcctcatccagcatcttcgagggctccagaaaggcagaagcacagagaggaacgtgtcagtggaagccctggcctctgctctgcagctgttagccagggagcagcaaagcacaggaagggtcgggcgctccctcccgacagaggactgtgagaatgagaaggagcaagctgtgcacaatgtagtccagctgctgccaggagtgggaaccttctacaacctgggcacagctttgtattatgctactcaaaactgcctgggcaaggccagggaacgaggccgagatggggccatagatctgggatatgaccttctgatgaccatggctgggatgtcaggggggcctatgggtctagcgatcagtgctgcacttaaacctgcattaaggtctggggttcagcagttgatccagtattaccaagatcagaaagacgcaaacatctctcagccggagaccaccaaggagggtttgagggccatctcagatgtgagtgacttggaagaaacaactactctggcttctttcatatcagaagtagtaagttcagctccctactgggggtgggccataatcaagagctatgacttagatcctggggctgggagtcttgagatataaaagaatgtggtaaccacagaattaataactgtactaccctgacaagctatatacatgtcttcaaaattttaatctgatttatccaggaggaaggctgtacagtaaaacgtaagaacgtaaatgtttgggtgttgaagtcacagggtttggtttcgaatctaggctccacttgttagagcctcggtgatcactgaatagtaacttctttcttgaactaagatcagttttgaagtttctaaaggagatagaatgattttaacctcaatgagttgccctgtaaatttaaaatgatacaatgaatctaaaatgcttatcacagtactttcaataaatagctattagccaggtgcggtggctcacgcctgtaatcccagcactgtgagaggctgaggcgggatgatcacctgaggtcaggagttcaagatcagcctgggcaacatggcgaaaccccgtctctacaataaatacaaaaaattatcctggcggagttatgcacgcttgtagtcccaactacctgggaggctgaggcgggagaatcacctgagcctgggaggtcgaggctgcagcgagccgagatcgcgccgctgcattccagcctgggtgacagagcgagaccatgtctcaaaaaaaataaaaataaaaaaaaattgttttcacaaaaaaaaaaaaaaaaaaaaaaaaaaaaaa

SEQ ID NO: 27 - DNA, 3653 bp, Homo sapiens
gcgccagcgccggcgggacccgagctgctaatgaggcagccatgactggccacttcatgtgctcctggagaagggcttgcaccagccgttttcaggaaagtcaagcagctgttgactcctgagtctgggtgaatttgtgtgaagagcataaggcgctgtttcttaaccaaaacgcttcctcttgcagtgcagatgggatgtgcttctccacaggaggccccacggcttccccacccctcagaggagcgccgtgcgtgcgtctgtgtggaggattggcagctcctgcagtcggcccttggtcctatttggcgacgcctctgccttccccttaattatacagtcatgagccgccctggaatcacggcagctccggatggatcctggatgccagaatgcagcctcagcacggggctgcaggataggagtgagcgaggggctgcagagccggcggccgcggtgggcaccatggagggggctgccctgggcagcacgggcatgagtctcaaggcccaggtttgagtaacaggtgttgagagcttacttacttttcctgagacacagtttcctcatctcgagagcacggaaaatcattctaacttcagaggattgttgtgaaagttaaatgagattaaagaggtaaagcccatgacgtgcttagctcgtgcttggctcttggtcaatgccagttagcgctgcattttctcccctctccctccctccttctctctttcttttcttctattctccattcctgttttctcccccaccccactccccaaagctctgcgttgagaaccagatgctgtctggtgggttagggccagaggaggaaaagctgcctgccgtgggctgcacccataccctcttcattccaatgacatgaggggaggggaaaggacagaggtagactgtcctcccctacctcctcctaatacaaatggaattcctggaactggaaaacaaagaatacccccataaaaataagacagtacttctggtgcggtgtaataaaggggaaagtaaccctcaatgtcaggaaactccgcacctcccagctcatatttgtgtggaggaaaagttaaatattaatttggactcaactgaatgtggacacaaacaatggtcaccaagtcccggaacaggttgtgtgagcctcttcaggggttcatccagcgctgttttggagaaatctctatttcaatttattcctatacgttagttactgaaaaacaacagacaatcgcaaaagcaagttgcccgttttgtgttccttgagcccaatcatgaagtgccgtcgtgactgggcctcatgacaaacaacttgtaataagtaacaacagagctcaggtcccagaccgcactgaagctctgtgagacctctcctcatctgtgcatgaacgagtgtctgactctggagcccagcctgctgcttcccagtctggtggtgaatcctccgtagtctgatggaggtttgctcttgttgcccaggctggagtgcaatggcacaatctcggctcactgcagcccctgcctcccaggctcaagcaattcttatgcctcagcctcctgagtagatggaactacagggcatggactggaactcccaatacccctgacatgggctgagtcaacgtggtcatgaacatgtgacaggaggcagcagaagttgcagaaaagagtgaggcacgtttgaaaaaggctgaaaaatgtttctgtccaggcaagggtgtgtgctgaatgactcaaggattttttggagagaattggagtgtctcaccagaggagaccacgtctgaagggctttgcatccctccttggacatgtctaatacctagcactcagaaagcatccagtaaatattcgtggaaagaaaggagtggagaaggggagaaaggggaaagggagtaggcgagagagaagaaagactctgcttcttgcccagggcctggcatggggcggaggcaaagcagtggggtcctcagctatgtcccactgtgagtgcacagcgagtcctgaccttcagagggtgcagcccgaggggccctggcctgtctgaagggtgcgccagccgagtggcctgctctgaccaccaggctcacccatgactacctgggtggctacagccagttcctgacaatgagtacagcactcagttatcggggcccttccacccacacgctgtccacttcctggggtactgctgtgggcatgcgagtgcttgctccccggggcactgctgtgggcatgcgagtgcttgctccccggggcactgctgtgggcatgtgagtgcttgctccccagctccactagtgacactggtggccccttgctggggccttccccgcctgctccgctccattaccgctgccgggctcctcacgtctctccttgctgcttcctgcactggggtgaggagagtggggctggtccccttgagaccggagaagctccaggcttttaaggaaaactgccagggacgaagagaagatatcacttccccacgtggttggcttccagattcagaaggaatgtctgtccttgtggattccgtaccagatgaccccagatgctgcctcagtactaggtccctgtggctctggagcctttgctgggtctgggcagtgtctcttcctctccagttcatccttgggtctcttcacccttgccaggggcaggcttcctggtgagaggtcgacctcctgcatgaaggctctcaagaggccagttcaaagccaagctccgggtctgtgcctgtggggctgctcctcgatcaggagatggtcactcccctcctggtctgtatctgtgggattctcctccatcaggagatggtctctcccctcctggtctatacccgtgggattctcctccatcaggagatggtcactccccatcctggtctatacccgtgggattctcctccattagatggtcactcccctcctggtctattcccgtggggctgctcctccatcaggaggtggtcactccccctcctggtctatacccgtggggctgctcctccatcaggagatagtcactccccctcctggtctatacccgtgggattctcctccatctggagatggtcactcccctcctggtctatacccatgggattctcctccatctggagatggtcactcccctcctggtctatacccatgggattctcctccatctggagatagtcactcccctcctggtctatacccgtggggttctcctcaatcaggaggtggtcactcccctcctggtctatacccgtgggattctcctccatcaggagatggtcactctccctcctggtctatacccgtggggctgctcctccatcaggagatggtcactcccctcctggtctatacctgtgggattctcctccctcagaagatggtcactccccctcctggtctatacccatgggattctcctccctcagaagatggtcactccccctcctggtctatacccgtggggctgctcctccatcaggagatggtcactctccctctcggttgctcagtccaaaaacaacctctctggaaaactgcgtggaatttttttttaaagaattgaaactagaactagcatttgatccagccatctgcctactgggaatacacccaaagaaaaataaatcattatatcag

SEQ ID NO: 28 - protein, 1214 aa, Homo sapiens
MVVPEKEQSWIPKIFKKKTCTTFIVDSTDPGGTLCQCGRPRTAHPAVAMEDAFGAAVVTVWDSDAHTTEKPTDAYGELDFTGAGRKHSNFLRLSDRTDPAAVYSLVTRTWGFRAPNLVVSVLGGSGGPVLQTWLQDLLRRGLVRAAQSTGAWIVTGGLHTGIGRHVGVAVRDHQMASTGGTKVVAMGVAPWGVVRNRDTLINPKGSFPARYRWRGDPEDGVQFPLDYNYSAFFLVDDGTHGCLGGENRFRLRLESYISQQKTGVGGTGIDIPVLLLLIDGDEKMLTRIENATQAQLPCLLVAGSGGAADCLAETLEDTLAPGSGGARQGEARDRIRRFFPKGDLEVLQAQVERIMTRKELLTVYSSEDGSEEFETIVLKALVKACGSSEASAYLDELRLAVAWNRVDIAQSELFRGDIQWRSFHLEASLMDALLNDRPEFVRLLISHGLSLGHFLTPMRLAQLYSAAPSNSLIRNLLDQASHSAGTKAPALKGGAAELRPPDVGHVLRMLLGKMCAPRYPSGGAWDPHPGQGFGESMYLLSDKATSPLSLDAGLGQAPWSDLLLWALLLNRAQMAMYFWEMGSNAVSSALGACLLLRVMARLEPDAEEAARRKDLAFKFEGMGVDLFGECYRSSEVRAARLLLRRCPLWGDATCLQLAMQADARAFFAQDGVQSLLTQKWWGDMASTTPIWALVLAFFCPPLIYTRLITFRKSEEEPTREELEFDMDSVINGEGPVGTADPAEKTPLGVPRQSGRPGCCGGRCGGRRCLRRWFHFWGAPVTIFMGNVVSYLLFLLLFSRVLLVDFQPAPPGSLELLLYFWAFTLLCEELRQGLSGGGGSLASGGPGPGHASLSQRLRLYLADSWNQCDLVALTCFLLGVGCRLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQRYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 29 - DNA, 4103 bp, Homo sapiens
aggctggaaagtggaggatccggtttgctctgggcgggtctggaagcagagccggcggagggagcgccggggccctgggctgcaggaggttgcggcggccgcggcagcatggtggtgccggagaaggagcagagctggatccccaagatcttcaagaagaagacctgcacgacgttcatagttgactccacagatccgggagggaccttgtgccagtgtgggcgcccccggaccgcccaccccgcagtggccatggaggatgccttcggggcagccgtggtgaccgtgtgggacagcgatgcacacaccacggagaagcccaccgatgcctacggagagctggacttcacgggggccggccgcaagcacagcaatttcctccggctctctgaccgaacggatccagctgcagtttatagtctggtcacacgcacatggggcttccgtgccccgaacctggtggtgtcagtgctggggggatcggggggccccgtcctccagacctggctgcaggacctgctgcgtcgtgggctggtgcgggctgcccagagcacaggagcctggattgtcactgggggtctgcacacgggcatcggccggcatgttggtgtggctgtacgggaccatcagatggccagcactgggggcaccaaggtggtggccatgggtgtggccccctggggtgtggtccggaatagagacaccctcatcaaccccaagggctcgttccctgcgaggtaccggtggcgcggtgacccggaggacggggtccagtttcccctggactacaactactcggccttcttcctggtggacgacggcacacacggctgcctggggggcgagaaccgcttccgcttgcgcctggagtcctacatctcacagcagaagacgggcgtgggagggactggaattgacatccctgtcctgctcctcctgattgatggtgatgagaagatgttgacgcgaatagagaacgccacccaggctcagctcccatgtctcctcgtggctggctcagggggagctgcggactgcctggcggagaccctggaagacactctggccccagggagtgggggagccaggcaaggcgaagcccgagatcgaatcaggcgtttctttcccaaaggggaccttgaggtcctgcaggcccaggtggagaggattatgacccggaaggagctcctgacagtctattcttctgaggatgggtctgaggaattcgagaccatagttttgaaggcccttgtgaaggcctgtgggagctcggaggcctcagcctacctggatgagctgcgtttggctgtggcttggaaccgcgtggacattgcccagagtgaactctttcggggggacatccaatggcggtccttccatctcgaagcttccctcatggacgccctgctgaatgaccggcctgagttcgtgcgcttgctcatttcccacggcctcagcctgggccacttcctgaccccgatgcgcctggcccaactctacagcgcggcgccctccaactcgctcatccgcaaccttttggaccaggcgtcccacagcgcaggcaccaaagccccagccctaaaagggggagctgcggagctccggccccctgacgtggggcatgtgctgaggatgctgctggggaagatgtgcgcgccgaggtacccctccgggggcgcctgggaccctcacccaggccagggcttcggggagagcatgtatctgctctcggacaaggccacctcgccgctctcgctggatgctggcctcgggcaggccccctggagcgacctgcttctttgggcactgttgctgaacagggcacagatggccatgtacttctgggagatgggttccaatgcagtttcctcagctcttggggcctgtttgctgctccgggtgatggcacgcctggagcctgacgctgaggaggcagcacggaggaaagacctggcgttcaagtttgaggggatgggcgttgacctctttggcgagtgctatcgcagcagtgaggtgagggctgcccgcctcctcctccgtcgctgcccgctctggggggatgccacttgcctccagctggccatgcaagctgacgcccgtgccttctttgcccaggatggggtacagtctctgctgacacagaagtggtggggagatatggccagcactacacccatctgggccctggttctcgccttcttttgccctccactcatctacacccgcctcatcaccttcaggaaatcagaagaggagcccacacgggaggagctagagtttgacatggatagtgtcattaatggggaagggcctgtcgggacggcggacccagccgagaagacgccgctgggggtcccgcgccagtcgggccgtccgggttgctgcgggggccgctgcggggggcgccggtgcctacgccgctggttccacttctggggcgcgccggtgaccatcttcatgggcaacgtggtcagctacctgctgttcctgctgcttttctcgcgggtgctgctcgtggatttccagccggcgccgcccggctccctggagctgctgctctatttctgggctttcacgctgctgtgcgaggaactgcgccagggcctgagcggaggcgggggcagcctcgccagcgggggccccgggcctggccatgcctcactgagccagcgcctgcgcctctacctcgccgacagctggaaccagtgcgacctagtggctctcacctgcttcctcctgggcgtgggctgccggctgaccccgggtttgtaccacctgggccgcactgtcctctgcatcgacttcatggttttcacggtgcggctgcttcacatcttcacggtcaacaaacagctggggcccaagatcgtcatcgtgagcaagatgatgaaggacgtgttcttcttcctcttcttcctcggcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcagcgttaccgcctcatccgggaattccactctcggcccgcgctggccccgccctttatcgtcatctcccacttgcgcctcctgctcaggcaattgtgcaggcgaccccggagcccccagccgtcctccccggccctcgagcatttccgggtttacctttctaaggaagccgagcggaagctgctaacgtgggaatcggtgcataaggagaactttctgctggcacgcgctagggacaagcgggagagcgactccgagcgtctgaagcgcacgtcccagaaggtggacttggcactgaaacagctgggacacatccgcgagtacgaacagcgcctgaaagtgctggagcgggaggtccagcagtgtagccgcgtcctggggtgggtggccgaggccctgagccgctctgccttgctgcccccaggtgggccgccaccccctgacctgcctgggtccaaagactgagccctgctggcggacttcaaggagaagcccccacaggggattttgctcctagagtaaggctcatctgggcctcggcccccgcacctggtggccttgtccttgaggtgagccccatgtccatctgggccactgtcaggaccacctttgggagtgtcatccttacaaaccacagcatgcccggctcctcccagaaccagtcccagcctgggaggatcaaggcctggatcccgggccgttatccatctggaggctgcagggtccttggggtaacagggaccacagacccctcaccactcacagattcctcacactggggaaataaagccatttcagaggaatcgtgaaaaaaa

SEQ ID NO: 30 - DNA, 3923 bp, Homo sapiens
acagaagaaatagcaagtgccgagaagctggcatcagaaaaacagaggggagatttgtgtggctgcagccgagggagaccaggaagatctgcatggtgggaaggacctgatgatacagaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 31 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 32 - DNA, 2707 bp, Homo sapiens
catctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 33 - protein, 772 aa, Homo sapiens
MNCYLLLRFMLGIPLLWPCLGATENSQTKKVKQPVRSHLRVKRGWVWNQFFVPEEMNTTSHHIGQLRSDLDNGNNSFQYKLLGAGAGSTFIIDERTGDIYAIQKLDREERSLYILRAQVIDIATGRAVEPESEFVIKVSDINDNEPKFLDEPYEAIVPEMSPEGTLVIQVTASDADDPSSGNNARLLYSLLQGQPYFSVEPTTGVIRISSKMDRELQDEYWVIIQAKDMIGQPGALSGTTSVLIKLSDVNDNKPIFKESLYRLTVSESAPTGTSIGTIMAYDNDIGENAEMDYSIEEDDSQTFDIITNHETQEGIVILKKKVDFEHQNHYGIRAKVKNHHVPEQLMKYHTEASTTFIKIQVEDVDEPPLFLLPYYVFEVFEETPQGSFVGVVSATDPDNRKSPIRYSITRSKVFNINDNGTITTSNSLDREISAWYNLSITATEKYNIEQISSIPLYVQVLNINDHAPEFSQYYETYVCENAGSGQVIQTISAVDRDESIEEHHFYFNLSVEDTNNSSFTIIDNQDNTAVILTNRTGFNLQEEPVFYISILIADNGIPSLTSTNTLTIHVCDCGDSGSTQTCQYQELVLSMGFKTEVIIAILICIMIIFGFIFLTLGLKQRRKQILFPEKSEDFRENIFQYDDEGGGEEDTEAFDIAELRSSTIMRERKTRKTTSAEIRSLYRQSLQVGPDSAIFRKFILEKLEEANTDPCAPPFDSLQTYAFEGTGSLAGSLSSLESAVSDQDESYDYLNELGPRFKRLACMFGSAVQSNN

SEQ ID NO: 34 - DNA, 6241 bp, Homo sapiens
atactgacagtgttgtggtactaaaagcacaagcgtctgtaactctgggcaatggggcacatcgagagtttgctgagaagactgtgaagcaaaaagaagaaagtttttcctactcttccttatgtgtccaacacgaagtttgctgttcagttttcacagaacttctagaagttgaagttacaaaggtatatagaaggtacacagaatcagaaaagattataaaagaaagcaagatttttgttagtgacgtcctgtttcctctgaagagtaatagttggaatcaaaagagtcaacgcaatgaactgttatttactgctgcgttttatgttgggaattcctctcctatggccttgtcttggagcaacagaaaactctcaaacaaagaaagtcaagcagccagtgcgatctcatttgagagtgaagcgtggctgggtgtggaaccaattttttgtaccagaggaaatgaatacgactagtcatcacatcggccagctaagatctgatttagacaatggaaacaattctttccagtacaagcttttgggagctggagctggaagtacttttatcattgatgaaagaacaggtgacatatatgccatacagaagcttgatagagaggagcgatccctctacatcttaagagcccaggtaatagacatcgctactggaagggctgtggaacctgagtctgagtttgtcatcaaagtttcggatatcaatgacaatgaaccaaaattcctagatgaaccttatgaggccattgtaccagagatgtctccagaaggaacattagttatccaggtgacagcaagtgatgctgacgatccctcaagtggtaataatgctcgtctcctctacagcttacttcaaggccagccatatttttctgttgaaccaacaacaggagtcataagaatatcttctaaaatggatagagaactgcaagatgagtattgggtaatcattcaagccaaggacatgattggtcagccaggagcgttgtctggaacaacaagtgtattaattaaactttcagatgttaatgacaataagcctatatttaaagaaagtttataccgcttgactgtctctgaatctgcacccactgggacttctataggaacaatcatggcatatgataatgacataggagagaatgcagaaatggattacagcattgaagaggatgattcgcaaacatttgacattattactaatcatgaaactcaagaaggaatagttatattaaaaaagaaagtggattttgagcaccagaaccactacggtattagagcaaaagttaaaaaccatcatgttcctgagcagctcatgaagtaccacactgaggcttccaccactttcattaagatccaggtggaagatgttgatgagcctcctcttttcctccttccatattatgtatttgaagtttttgaagaaaccccacagggatcatttgtaggcgtggtgtctgccacagacccagacaataggaaatctcctatcaggtattctattactaggagcaaagtgttcaatatcaatgataatggtacaatcactacaagtaactcactggatcgtgaaatcagtgcttggtacaacctaagtattacagccacagaaaaatacaatatagaacagatctcttcgatcccactgtatgtgcaagttcttaacatcaatgatcatgctcctgagttctctcaatactatgagacttatgtttgtgaaaatgcaggctctggtcaggtaattcagactatcagtgcagtggatagagatgaatccatagaagagcaccatttttactttaatctatctgtagaagacactaacaattcaagttttacaatcatagataatcaagataacacagctgtcattttgactaatagaactggttttaaccttcaagaagaacctgtcttctacatctccatcttaattgccgacaatggaatcccgtcacttacaagtacaaacacccttaccatccatgtctgtgactgtggtgacagtgggagcacacagacctgccagtaccaggagcttgtgctttccatgggattcaagacagaagtcatcattgctattctcatttgcattatgatcatatttgggtttatttttttgactttgggtttaaaacaacggagaaaacagattctatttcctgagaaaagtgaagatttcagagagaatatattccaatatgatgatgaagggggtggagaagaagatacagaggcctttgatatagcagagctgaggagtagtaccataatgcgggaacgcaagactcggaaaaccacaagcgctgagatcaggagcctatacaggcagtctttgcaagttggccccgacagtgccatattcaggaaattcattctggaaaagctcgaagaagctaatactgatccgtgtgcccctccttttgattccctccagacctacgcttttgagggaacagggtcattagctggatccctgagctccttagaatcagcagtctctgatcaggatgaaagctatgattaccttaatgagttgggacctcgctttaaaagattagcatgcatgtttggttctgcagtgcagtcaaataattagggctttttaccatcaaaatttttaaaagtgctaatgtgtattcgaacccaatggtagtcttaaagagttttgtgccctggctctatggcggggaaagccctagtctatggagttttctgatttccctggagtaaatactccatggttattttaagctacctacatgctgtcattgaacagagatgtggggagaaatgtaaacaatcagctcacaggcatcaatacaaccagatttgaagtaaaataatttaggaagatattaaaagtagatgagaggacacaagatgtagtcgatccttatgcgattatatcattatttacttaggaaagagtaaaaataccaaacgagaaaatttaaaggagcaaaaatttgcaagtcaaatagaaatgtacaaatcgagataacatttacatttctatcatattgacatgaaaattgaaaatgtatagtcagagaaattttcatgaattattccatgaagtattgtttcctttatttaaaaaaaaaaaaaaaaaaaaagaatgctaggtaatcttcgtagaaaactagaaagtatgataaacaacagttggaggaatccatggaaaatagacgagaaaatgtaaataaggctttctggggatcacagaacttttgtatgaataaaagccttataaaaccagctctggcatatgactaaaatattcctctactatttttcaatgtcatctaatgcaaacgctcaacctttttaaatggttaatgtgagaaaggcactgaattaagatatcttagtgttattttatttactcaattttcatctttcacactgtaagaataaaatgtttttgtggttaagttcctgatactatatagagataaactttaaaactttaacacctaggataatgtgggtgattacataaatgtttaaaaacatttaccttatatggaaagcagatttctgactaagatgccatgattcattctagtttgatattttaaaactttgccaagagcactctttaaaactgatttcaaatttgaacagatacttggtgtatacaaattaatcagagttatatagtataaatttgtattatttttgatattacaactttgtattaaaaaggaaaatatatttaggtgtttgtcatagattttttttcatatgcaactcaaaacacattcttatgtcaaaatacaatagcaaaatacaagtcaatatttactcataattaaatgcaaactttatgaaactggtggaaagtgtggaaaaatttaagtaaatttttacctctactaaaaattatttagacaacttaaaggaatactggattcaaacttcagttactattcactgtaaatattatgatcaactgaactgacttaccattcatgggataaaacatgatacaattctcagtatttgcttttctgttaagagcttggaatatgactgatgcttcagtgtcatttttttattattgaatataaaattaggtgcaatagatacatttattgtagccttaagaaggaaatcaatgctaagctgttgggcataaaactttacagtaacattagataacaagataataaaacactttaattttaatcgaccttttaagaatgatctttattattctatccaaatcaaggtagacttcaaaatttatggtttgaacaggaagaaagtagtgaatacaaaattcttaaaattggttcatatgatcatttttttattaatagcatgtgagactaagctgaaccaaactggccatatatgttaaatcaaataatttgagtaattttgaaatattgatgaagtagttaaatggttataatatattttattggtactttgaaatttaaacatctatatcacataataaagaaagtttttcatcaatctgaaacaggcaccattaaattggtgacacaaatattatcatttcacaaaagcttgagaaaaccagatttgaagtgaaataatttaggaagatattaaaagtggatgagaggacataagatgtagtcaatccttaacgcaatgataccattattaggaaagaggaaaaataccaaacaagagaaaatttaaaggagcaaaaatttgcaagtcaaatagaaatgcacaaatcgagagaacatttacatttctatcacattgacatgaaaattgaaaatgtatagtcagagaaattttcatgaattattccatgaagtgttgtttcctttatttaaaaagaatgctaggtaatcttagtagaaaactaaaaagtatgataaacaacagatggaagaattcatggaaaatagacgagaaaaagtaaataaggctttctggagatgacggaaattttgtatgaataaaagccttataaacaatgaatacatttggataaacaactcattaaaatgcacattaatggaatacctgtacaaatagttttaatacttgtgtccagtggcactaaagagatatattaaaatagattctggttgagttgattataagattgtcaatcataggatacacagggaaagcacagacacgattgctcatctctttggtagtctaaaatatgttccttttaaccacatcaaaagatactatctgattctaatacagaaggtgcaacttcctaggaattatactttgtattatattcaataattattttatgttttgcatcacatgggtgttgcaagaacacagctcttaaactaaaaaggcattacattaagaatttatttttgtaaattttaataaactctaaattaatgcaaaatatttatgaaaatagcacagcttcacattaatacccacagtacttaaatatctcccttagttgactcacagctttgtatgtaagcttcaaatttttaatggtaaaaataagaaaattaggactagcgaactgctagttgttccctcagtgcatgtgcttgagcctaacagatctttgtttgaattagttactttcaattaagcgaacatataatatttgttaaactgcttaatctttccagatctcactttgtaaaatgacgtccaatcaacctatttcacatggttgttatgaagaattaataataagatatgtaaaaccagcagcaccaatagtgcctgacaaaatataactgctcaataaataacagtgaattattataacaacagctaatggtattctattggaacacatggtctgtttcttttccaattctgagtgatctcaacagcctcacggcacattgaaaataaaagtcaacttcctcaccttccccataggccatggactttcccatctctacccacttgtgcagtttctcctctttcctctctcctcctcagttactaaagcaatcttccttcaaacacttctattttccattacatcacttcattcaggttggtgatcagctgcaacttcctcagaaagaccactgctgcttccagtgaccctggctttgtcttgctttgtattagattactcagctccctggtattattgttgtctttatcgcttattagaatgtaacccttaatgggcaagaaattgtcagtcttgttcatttatcatcgtataaaaaatgtttctagcacacagaaagtgaccaataaatatttctggaattaatgta

SEQ ID NO: 35 - protein, 320 aa, Homo sapiens
MSSCNFTHATFVLIGIPGLEKAHFWVGFPLLSMYVVAMFGNCIVVFIVRTERSLHAPMYLFLCMLAAIDLALSTSTMPKILALFWFDSREISFEACLTQMFFIHALSAIESTILLAMAFDRYVAICHPLRHAAVLNNTVTAQIGIVAVVRGSLFFFPLPLLIKRLAFCHSNVLSHSYCVHQDVMKLAYADTLPNVVYGLTAILLVMGVDVMFISLSYFLIIRTVLQLPSKSERAKAFGTCVSHIGVVLAFYVPLIGLSVVHRFGNSLHPIVRVVMGDIYLLLPPVINPIIYGAKTKQIRTRVLAMFKISCDKDLQAVGGK

SEQ ID NO: 36 - DNA, 2799 bp, Homo sapiens
ctcacacaccctgaagacacagtgagttagcaccaccaccaggaattggccttttagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaacttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacggagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacctgagtataagataaaataaaattttattttaaattttaaaaaaaaaaaaaaaaaa

SEQ ID NO: 37 - protein, 2224 aa, Homo sapiens
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 38 - DNA, 9179 bp, Homo sapiens
gcaagaactgcaggggaggaggacgctgccacccacagcctctagagctcattgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaactcatcattgaatcaggaagaagaagagttcaatcttactgccctagctctggagaatggcactgaattcgtttcttcaaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaaaagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccctttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatacaagaagcttttataatgtaactccttgctaccagtaagtaagataatggctattacttctgcattaatttgaatacaggtaggaaaatatcaagaaccaacaagaaaagggcttatctttcttaatgattgaaaatgctatgaagtaatatttatgtagttaaaatgcttcattataactcttttaaatcctttacacactagtaaaacagatattactttaaataataattgatagacctggataactttcacaaacacatgattttttaatggtttttcttgagtgaagagaaaaacaatattatcaaatgaaataagtacttaaaatatcctgtctttcccatataacaatgatttttctgactttccatgagtaaaaaaacagccaagcatctttccagtagccccattgaaattgtgaatccgtcctggtctccctaaggactgcacacattgatattcaaggttggtggtcattagatatggaacagaactgaaataaccatggtagaactgaatgtgtaatgttggctttattctagctggtactacatggcacacagtttcaaaacataatttcacctactggaaagctcagacctgtaaaacagagcatgggaactgctggtctaaatgcagttgttcctgctcaaagagacctctggccaaactggcaagcagttaaagttttctttcagggccttcctctctatggcctcaacttcctcctctctcttcttccagcaacttcccctttcatcattcctttccctggggacttggcattcagtgatcctgtagatattgcacaactggggaacctttagacatccttaaaatcacatgagatagacagtcatttggggtgtctgaaataaaccaccccaaaacttagtgttaaaagagcaaccaaaaaaaatttatgtgagattatggatttgttacttagcttgatttaatcatcctgtaacgtgtacatatatcaaaatgttatgtataccataaatatataaaattttatcaacgaaattcataacaatctctcagaccacagagaaatcaaattagaactgaggactaagaaactcactcgaaaccacacaactacatggaaactgaacaacctgctcctgaatgactactgggtaaataatgaaattaaggcagaaataaataagttccttaaaaccaatgagaacaaagagacaacataccagaatctctaggagacagggctttgcttttgctgcattctattcgttgtgaacacaaattacaggccagtctcgattcagtgtagaagggaactgcataaggaccacataccaggaggcataattcactgggagcatctttagaaactaccagagttacctgttgcccataccagtggggtaagccctatgaatgtatatgagagtttcaaacatccacaaaacattggctttctaatattcgtattcccactattcctttcttttcatgattcatgtcattgtcccatcaacatttctaagatttccattccgttaagagcaaaagagaatgttggaaggtgggggaaaacatttctttgttttctacagggccagcttcttggatgtgtgtgatctgttcagttgcaaagggtcacatgctcagaaggaccgcatgctaaatttaatgctttgcagttaccctcttgaaatcctttattttttaagaaggaattcgacatttccatttttcaatgagccccacaaattacgcagctagtcctgggcttctctactctgaaattgggcaggatctctcttgatctagaatttactaaggcataataggggcaagaaaatcttatgaaataatggggggtagggaagagatgggaatggagcatgagatccagcttcgttattctctacttgagaaaaataaggccccaaagattaaacaacttgcccaaggatattgcttgttagtgtcagaactgaaaccagaaaccaaatgatcatatccctagacttttagtctgctttctcttccataaaatgaaacttataatgtttctaatccattgctcagacaggtagacatgaatattaattgataatgactattaattgatctggaaaatacttgtttggggatcaataatatgtttgggctattatctaatgctgtgtagaaatattaaaacccctgttattttgaaataaaaaagatacccactttt

SEQ ID NO: 39 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 40 - DNA, 3195 bp, Homo sapiens
acagcatggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggaggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctgttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctgagcggaggggagggggcgcccaggaggggtggggaggggggcgaggagacccaagtgcagcgaagggacacttgatgggctgaggttcccaccccttcacagtgttgattgctattagcatgataatgaactcttaatggtatccattagctgggacttaaatgactcacttagaacaaagtacctggcattgaagcctcccagacccagccccttttcctccattgatgtgcggggagctcctcccgccacgcgttaatttctgttggctgaggagggtggactctgcggcgtttccagaacccgagatttggagccctccctggctgcacttggctgggtttgcagtcagatacacagatttcacctgggagaacctctttttctccctcgactcttcctacgtaaactcccacccctgacttaccctggaggaggggtgaccgccacctgatgggattgcacggtttgggtattcttaatgaccaggcaaatgccttaagtaaacaaacaagaaatgtcttaattatacaccccacgtaaatacgggtttcttacattagaggatgtatttatataattatttgttaaattgtaaaaaaaaaaagtgtaaaatatgtatatatccaaagatatagtgtgtacatttttttgtaaaaagtttagaggcttacccctgtaagaacagatataagtattctattttgtcaataaaatgacttttgataaatgatttaaccattgccctctcccccgcctcttctgagctgtcacctttaaagtgcttgctaaggacgcatggggaaaatggacattttctggcttgtcattctgtacactgaccttaggcatggagaaaattacttgttaaactctagttcttaagttgttagccaagtaaatatcattgttgaactgaaatcaaaattgagtttttgcaccttccccaaagacggtgtttttcatgggagctcttttctgatccatggataacaactctcactttagtggatgtaaatggaacttctgcaaggcagtaattccccttaggccttgttatttatcctgcatggtatcactaaaggtttcaaaaccctgaaaaaaaa

SEQ ID NO: 41 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 42 - DNA, 1318 bp, Homo sapiens
agcccagtcgccgccgcccgcccacaaagccacaggcaggtgcaggcgcagccgcggcgagagcgtatggagccgagccgttagcgcgcgccgtcggtgagtcagtccgtccgtccgtccgtccgtcggggcgccgcagctcccgccaggcccagcggccccggcccctcgtctccccgcacccggagccacccggtggagcgggccttgccgcggcagccatgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtctaagggacagacgcagggagaccccaccaccaccaccaccaccaacaccaccaccaccaccgcgagctggagcgcgcaccaggccatccagcgtgcagccttgcctcggaggccagcccacccccagaagccaggaagcccccgcgctggactggggcagcttccccagcagccacggctttgcgggccgggcagtcgacttcggggcccagggaccaacctgcatggactgtgaaacctcacccttctggagcacggggcctgggtgaccgccaatacttgaccaccccgtcgagccccatcgggccgctgcccccatgctcgcgctgggcagggaccggcagccctggaaggggcacttgatatttttcaataaaagcctttcgttttgcaaaaaaaaaaaaaaaaaaaaaaaaaaaaaaaaaaaaaaaaaaaaa

SEQ ID NO: 43 - protein, 264 aa, Homo sapiens
MIMSSYLMDSNYIDPKFPPCEEYSQNSYIPEHSPEYYGRTRESGFQHHHQELYPPPPPRPSYPERQYSCTSLQGPGNSRGHGPAQAGHHHPEKSQSLCEPAPLSGASASPSPAPPACSQPAPDHPSSAASKQPIVYPWMKKIHVSTVNPNYNGGEPKRSRTAYTRQQVLELEKEFHYNRYLTRRRRIEIAHSLCLSERQIKIWFQNRRMKWKKDHRLPNTKVRSAPPAGAAPSTLSAATPGTSEDHSQSATPPEQQRAEDITRL

SEQ ID NO: 44 - DNA, 2328 bp, Homo sapiens
aactttttattgtggtttgtccgttccgagcgctccgcagaacagtcctccctgtaagagcctaaccattgccagggaaacctgccctgggcgctcccttcattagcagtattttttttaaattaatctgattaataattatttttcccccatttaattttttttcctcccaggtggagttgccgaagctgggggcagctggggagggtggggatgggaggggagagacagaagttgagggcatctctctcttccttcccgaccctctggcccccaaggggcaggaggaatgcaggagcaggagttgagcttgggagctgcagatgcctccgcccctcctctctcccaggctcttcctcctgcccccttcttgcaactctccttaattttgtttggcttttggatgattataattatttttatttttgaatttatataaagtatatgtgtgtgtgtgtggagctgagacaggctcggcagcggcacagaatgagggaagacgagaaagagagtgggagagagagaggcagagagggagagagggagagtgacagcagcgctcgcgggggctcaacccccagacctccagaaatgacgtcagaatcatttgcatcccgctgcctctacctgcctggtccagctgggaccctgcctcgccggccgcatggccagagggttggaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagttttaaaaaaaaaaaaaaaaaaaaaaa

SEQ ID NO: 45 - protein, 264 aa, Homo sapiens; identical to SEQ ID NO: 43

SEQ ID NO: 46 - DNA, 2459 bp, Homo sapiens
acacacgtgttacatggatacagctcctagccagaagcaagcaggctctacccacacaggcctccctcttagctagcgggggctcaacccccagacctccagaaatgacgtcagaatcatttgcatcccgctgcctctacctgcctggtccagctgggaccctgcctcgccggccgcatggccagagggttgggtgagtgtgtatggggaagaggggctggactctggtatccttggatggggggcactccaggctctccagcctcctcggctcagcctgggcccctccccatccaacatccactccagtcctcattcaacttcctcttcctgcgaaagaggggcgctgccccgtgacctacacagactgagacacgatcgccatgaatggagacctctggaaaagctcaggagccgaggcccacggggcccagcagaggcctgaggggagaccctgggcgggggctgaatcactgcctcccgacagtcccccaatgcccgggctttggaggggagccgggagcttcccatctccttttgcaggggagggttgtcagtctggcgggatgtgcactgggggcactccaacctctgctagctaaccccacatcaccacccacccccgcctcccagcaccaccaccaccacacacacaaaaaaattggatacattttgaataaagcgattcggttccttatccggggactgggttgctccgtgtgattggccggaggagtcacatggtgaaagtaactttacagggtcgctagctagtaggagggctttatggagcagaaaaacgacaaagcgagaaaaattattttccactccagaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagtttta

SEQ ID NO: 47 - protein, 2120 aa, Homo sapiens
MRSFKRVNFGTLLSSQKEAEELLPALKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKMQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSSPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLRLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPEIMRTIPEEELTDNWRKMSFEILRGSDGEDSASGGKTPAPGPEAASGEWELLRLDSSKKKLPSPCPDKESDKDLGPRLRLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVRGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 48 - DNA, 6641 bp, Homo sapiens
ggttacattttggatcctcgcggagtactggtcaggcggttaagtcctgtacctaggaaagagggcgagctctggggcgctctccggtgtcatgaggagcttcaaaagagtcaactttgggactctgctaagcagccagaaggaggctgaagagttgctgcccgccttgaaggagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggttggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatgcaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgcgcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccggctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcgtggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctccaaggttagatttaatccttaggataactcttttaaagtgattttccccagtgttttatatgaaacatttccttttgatttaacctcagtataataaagatacatcatttaaaccctgaaaaaaaaaaaaaaaaaa

SEQ ID NO: 49 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 50 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 51 - protein, 5058 aa, Homo sapiens
  MOD_RES (283)..(283): Any amino acid
MGHAGCQFKALLWKNWLCRLRNPVLFLAEFFWPCILFVILTVLRFQEPPRYRDICYLQPRDLPSCGVIPFVQSLLCNTGSRCRNFSYEGSMEHHFRLSRFQTAADPKKVNNLAFLKEIQDLAEEIHGMMDKAKNLKRLWVERSNTPDSSYGSSFFTMDLNKTEEVILKLESLHQQPHIWDFLLLLPRLHTSHDHVEDGMDVAVNLLQTILNSLISLEDLDWLPLNQTFSQVSELVLNVTISTLTFLQQHGVAVTEPVYHLSMQNIVWDPQKVQYDLKSQFGFXDLHTEQILNSSAELKEIPTDTSLEKMVCSVLSSTSEDEAEKWGHVGGCHPKWSEAKNYLVHAVSWLRVYQQVFVQWQQGSLLQKTLTGMGHSLEALRNQFEEESKPWKVVEALHTALLLLNDSLSADGPKDNHTFPKILQHLWKLQSLLQNLPQWPALKRFLQLDGALRNAIAQNLHFVQEVLICLETSANDFKWFELNQLKLEKDVFFWELKQMLAKNAVCPNGRFSEKEVFLPPGNSSIWGGLQGLLCYCNSSETSVLNKLLGSVEDADRILQEVITWHKNMSVLIPEEYLDWQELEMQLSEASLSCTRLFLLLGADPSPENDVFSSDCKHQLVSTVIFHTLEKTQFFLEQAYYWKAFKKFIRKTCEVAQYVNMQESFQNRLLAFPEESPCFEENMDWKMISDNYFQFLNNLLKSPTASISRALNFTKHLLMMEKKLHTLEDEQMNFLLSFVEFFEKLLLPNLFDSSIVPSFHSLPSLTEDILNISSLWTNHLKSLKRDPSATDAQKLLEFGNEVIWKMQTLGSHWIRKEPKNLLRFIELILFEINPKLLELWAYGISKGKRAKLENFFTLLNFSVPENEILSTSFNFSQLFHSDWPKSPAMNIDFVRLSEAIITSLHEFGFLEQEQISEALNTVYAIRNASDLFSALSEPQKQEVDKILTHIHLNVFQDKDSALLLQIYSSFYRYIYELLNIQSRGSSLTFLTQISKHILDIIKQFNFQNISKAFAFLFKTAEVLGGISNVSYCQQLLSIFNFLELQAQSFMSTEGQELEVIHTTLTGLKQLLIIDEDFRISLFQYMSQFFNSSVEDLLDNKCLISDNKHISSVNYSTSEESSFVFPLAQIFSNLSANVSVFNKFMSIHCTVSWLQMWTEIWETISQLFKFDMNVFTSLHHGFTQLLDELEDDVKVSKSCQGILPTHNVARLILNLFKNVTQANDFHNWEDFLDLRDFLVALGNALVSVKKLNLEQVEKSLFTMEAALHQLKTFPFNESTSREFLNSLLEVFIEFSSTSEYIVRNLDSINDFLSNNLTNYGEKFENIITELREAIVFLRNVSHDRDLFSCADIFQNVTECILEDGFLYVNTSQRMLRILDTLNSTFSSENTISSLKGCIVWLDVINHLYLLSNSSFSQGRLQNILGNFRDIENKMNSILKIVTWVLNIKKPLCSSNGSHINCVNIYLKDVTDFLNIVLTTVFEKEKKPKFEILLALLNDSTKQVRMSINNLTTDFDFASQSNWRYFTELILRPIEMSDEIPNQFQNIWLHLITLGKEFQKLVKGIYFNILENNSSSKTENLLNIFATSPKEKDVNSVGNSIYHLASYLAFSLSHDLQNSPKIIISPEIMKATGLGIQLIRDVFNSLMPVVHHTSPQNAGYMQALKKVTSVMRTLKKADIDLLVDQLEQVSVNLMDFFKNISSVGTGNLVVNLLVGLMEKFADSSHSWNVNHLLQLSRLFPKDVVDAVIDVYYVLPHAVRLLQGVPGKNITEGLKDVYSFTLLHGITISNITKEDFAIVIKILLDTIELVSDKPDIISEALACFPVVWCWNHTNSGFRQNSKIDPCNVHGLMSSSFYGKVASILDHFHLSPQGEDSPCSNESSRMEITRKVVCIIHELVDWNSILLELSEVFHVNISLVKTVQKFWHKILPFVPPSINQTRDSISELCPSGSIKQVALQIIEKLKNVNFTKVTSGENILDKLSSLNKILNINEDTETSVQNIISSNLERTVQLISEDWSLEKSTHNLLSLFMMLQNANVTGSSLEALSSFIEKSETPYNFEELWPKFQQIMKDLTQDFRIRHLLSEMNKGIKSINSMALQKITLQFAHFLEILDSPSLKTLEIIEDFLLVTKNWLQEYANEDYSRMIETLFIPVTNESSTEDIALLAKAIATFWGSLKNISRAGNFDVAFLTHLLNQEQLTNFSVVQLLFENILINLINNLAGNSQEAAWNLNDTDLQIMNFINLILNHMQSETSRKTVLSLRSIVDFTEQFLKTFFSLFLKEDSENKISLLLKYFHKDVIAEMSFVPKDKILEILKLDQFLTLMIQDRLMNIFSSLKETIYHLMKSSFILDNGEFYFDTHQGLKFMQDLFNALLRETSMKNKTENNIDFFTVVSQLFFHVNKSEDLFKLNQDLGSALHLVRECSTEMARLLDTILHSPNKDFYALYPTLQEVILANLTDLLFFINNSFPLRNRATLEITKRLVGAISRASEESHVLKPLLEMSGTLVMLLNDSADLRDLATSMDSIVKLLKLVKKVSGKMSTVFKTHFISNTKDSVKFFDTLYSIMQQSVQNLVKEIATLKKIDHFTFEKINDLLVPFLDLAFEMIGVEPYISSNSDIFSMSPSILSYMNQSKDFSDILEEIAEFLTSVKMNLEDMRSLAVAFNNETQTFSMDSVNLXEEILGCLVPINNITNQMDFLYPNPISTHSGPQDIKWEIIHEVILFLDKILSQNSTEIGSFLKMVICLTLEALWKNLKKDNWNVSNVLMTFTQHPNNLLKTIETVLEASSGIKSDYEGDLNKSLYFDTPLSQNITHHQLEKAIHNVLSRIALWRKGLRFNNSEWITSTRTLFQPLFEIFIKATTGKNVTSEKEERTEKEMIDFPYSFKPFFCLEKYLGGLFVLTKYWQQIPLTDQSVVEICEVFQQTVKPSEAMEMLQKVKMMVVRVLTIVAENPSWTKDILCATLSCKQNGIRHLILSAIQGVTLAQDHFQEIEKIWSSPNQLNCESLSKNLSSTLESFKSSLENATGQDCTSQPRLETVQQHLYMLAKSLEETWSSGNPIMTFLSNFTVTEDVKIKDLMKNITKLTEELRSSIQISNETIHSILEANISHSKVLFSALTVALSGKCDQEILHLLLTFPKGEKSWIAAEELCSLPGSKVYSLIVLLSRNLDVRAFIYKTLMPSEANGLLNSLLDIVSSLSALLAKAQHVFEYLPEFLHTFKITALLETLDFQQVSQNVQARSSAFGSFQFVMKMVCKDQASFLSDSNMFINLPRVKELLEDDKEKFNIPEDSTPFCLKLYQEILQLPNGALVWTFLKPILHGKILYTPNTPEINKVIQKANYTFYIVDKLKTLSETLLEMSSLFQRSGSGQMFNQLQEALRNKFVRNFVENQLHIDVDKLTEKLQTYGGLLDEMFNHAGAGRFRFLGSILVNLSSCVALNRFQALQSVDILETKAHELLQQNSFLASIIFSNSLFDKNFRSESVKLPPHVSYTIRTNVLYSVRTDVVKNPSWKFHPQNLPADGFKYNYVFAPLQDMIERAIILVQTGQEALEPAAQTQAAPYPCHTSDLFLNNVGFFFPLIMMLTWMVSVASMVRKLVYEQEIQIEEYMRMMGVHPVIHFLAWFLENMAVLTISSATLAIVLKTSGIFAHSNTFIVFLFLLDFGMSVVMLSYLLSAFFSQANTAALCTSLVYMISFLPYIVLLVLHNQLSFVNQTFLCLLSTTAFGQGVFFITFLEGQETGIQWNNMYQALEQGGMTFGWVCWMILFDSSLYFLCGWYLSNLIPGTFGLRKPWYFPFTASYWKSVGFLVEKRQYFLSSSLFFFNENFDNKGSSLQNREGELEGSAPGVTLVSVTKEYEGHKAVVQDLSLTFYRDQITALLGTNGAGKTTIISMLTGLHPPTSGTIIINGKNLQTDLSRVRMELGVCPQQDILLDNLTVREHLLLFASIKAPQWTKKELHQQVNQTLQDVDLTQHQHKQTRALSGGLKRKLSLGIAFMGMSRTVVLDEPTSGVDPCSRHSLWDILLKYREGRTIIFTTHHLDEAEALSDRVAVLQHGRLRCCGPPFCLKEAYGQGLRLTLTRQPSVLEAHDLKDMACVTSLIKIYIPQAFLKDSSGSELTYTIPKDTDKACLKGLFQALDENLHQLHLTGYGISDTTLEEVFLMLLQDSNKKSHIALGTESELQNHRPTGHLSGYCGSLARPATVQGVQLLRAQVAAILARRLRRTLRAGKSTLADLLLPVLFVALAMGLFMVRPLATEYPPLRLTPGHYQRAETYFFSSGGDNLDLTRVLLRKFRDQDLPCADLNPRQKNSSCWRTDPFSHPEFQDSCGCLKCPNRSASAPYLTNHLGHTLLNLSGFNMEEYLLAPSEKPRLGGWSFGLKIPSEAGGANGNISKPPTLAKVWYNQKGFHSLPSYLNHLNNLILWQHLPPTVDWRQYGITLYSHPYGGALLNEDKILESIRQCGVALCIVLGFSILSASIGSSVVRDRVIGAKRLQHISGLGYRMYWFTNFLYDMLFYLVSVCLCVAVIVAFQLTAFTFRKNLAATALLLSLFGYATLPWMYLMSRIFSSSDVAFISYVSLNFIFGLCTMPITIMPRLLAIISKAKNLQNIYDVLKWVFTIFPQFCLGQGLVELCYNQIKYDLTHNFGIDSYVSPFEMNFLGWIFVQLASQGTVLLLLRVLLHWDLLRWPRGHSTLQGTVKSSKDTDVEKEEKRVFEGRTNGDILVLYNLSKHYRRFFQNIIAVQDISLGIPKGECFGLLGVNGAGKSTTFKMLNGEVSLTSGHAIIRTPMGDAVDLSSAGTAGVLIGYCPQQDALDELLTGWEHLYYYCSLRGIPRQCIPEVAGDLIRRLHLEAHADKPVATYSGGTKRKLSTALALVGKPDILLLDEPSSGMDPCSKRYLWQTIMKEVREGCAAVLTSHSMEECEALCTRLAIMVNGSFKCLGSPQHIKNRFGDGYTVKVWLCKEANQHCTVSDHLKLYFPGIQFKGQHLNLLEYHVPKRWGCLADLFKVIENNKTFLNIKHYSINQTTLEQVFINFASEQQQTLQSTLDPSTDSHHTHHLPI

SEQ ID NO: 52 - DNA, 17209 bp, Homo sapiens
ggactgagagcagggagcagcaggcatggggcatgccgggtgccagttcaaagccctgctgtggaagaattggctctgcagactcaggaacccggtccttttccttgctgaattcttctggccttgtatcctgtttgtaattctgacagttcttcgttttcaagaacctcccagatacagagacatttgttatttgcagccccgagatctacccagctgtggtgttatcccctttgttcaaagccttctttgtaacactggatcaaggtgtaggaacttcagctatgaagggtcaatggagcatcattttcgtttgtctaggttccaaactgcagctgaccccaagaaagtcaacaacctggcctttttaaaagagatacaagacctggcagaggaaattcatggaatgatggacaaggcaaaaaacttaaaaagactttgggtagaacgatccaacactccagattcttcttatggttccagtttttttacaatggatctcaataagaccgaggaggtaatattgaaactggaaagcctccatcagcagcctcatatctgggattttctacttttactgccgagactacacacaagccatgatcatgtggaagatggcatggatgttgcagtgaaccttctccagaccattttgaattccttaatatccctagaagatttagattggcttccactcaaccaaactttttcccaggtttctgaacttgtactgaatgtgaccatttcgacactgacatttctgcagcaacatggagtagcagtcaccgagccagtttaccacctgtccatgcagaatatagtgtgggatccacagaaagtccagtatgatctcaaatcccagtttggctttgrtgatcttcacacggaacagatcctgaactcttcagctgaactgaaggagattcccacagacacttccttggagaagatggtgtgttcagtcttgtctagcacatcagaggatgaagctgagaaatggggccacgttggaggctgccaccctaagtggtcagaagccaaaaactatcttgtccatgcagtcagctggctgcgagtctaccaacaggtgtttgttcagtggcaacagggtagcctgcttcagaagacactcacaggcatgggccatagtctggaggctctcaggaatcagtttgaagaagagagcaagccctggaaggtggtggaagctctgcacactgcactgctcctgctgaatgacagcttgtcagcagatggcccaaaagataatcatacatttccaaagatattacagcatctgtggaaattgcaaagcttgctgcaaaacctgccccagtggccggcactgaagagatttcttcagcttgatggagctctcagaaatgcgatagctcagaatttacattttgtccaagaagtcctcatttgcctggagacatcagctaatgattttaaatggtttgaacttaaccaattgaaactggaaaaggatgtgttcttttgggagctgaaacagatgttggcgaagaatgctgtctgcccgaatggtcgtttctctgagaaggaggtctttttgccgcctggaaactccagcatatggggtggtctccagggactgttgtgctattgtaactcctctgagacgagtgttttaaacaagctacttggttcagtagaggatgctgatcgtattttgcaagaggtcattacttggcacaaaaatatgtcagttttaatacctgaagaatatttggactggcaggaacttgagatgcagctgtcagaagcaagcctttcctgtactcggctcttcctgctgctgggagctgatccctctcctgagaatgatgtcttttctagtgactgtaagcaccagcttgtctccacagtgatatttcatacacttgaaaaaacacaatttttcctggaacaagcatattattggaaagccttcaaaaagtttatcaggaagacttgcgaagtggcccaatatgtaaatatgcaagagagtttccagaacagactattggcttttcctgaggaatctccttgttttgaagaaaacatggattggaaaatgatcagtgataattattttcaatttttgaataacttactcaagtctccaacagcttccatatccagggctttaaatttcacaaagcaccttctaatgatggaaaagaagttgcacacccttgaggatgaacaaatgaactttcttttatcatttgtggaattttttgagaaattattgttgcctaatctttttgactcctccattgttcccagtttccacagcctcccatctctcacagaggatattctgaatataagttctctgtggacaaatcatttaaaaagtttaaagagagacccatctgccactgatgctcagaaactcttggaatttggcaacgaagtgatttggaaaatgcagactctcggaagtcactggataaggaaggaaccaaaaaatcttttgagattcatagaattaatactttttgaaattaatcccaaattactagaattatgggcctatggcatttcaaaaggaaaaagagctaaattggaaaacttctttacacttttaaatttttctgttccagaaaatgagattctgagtacaagttttaacttttcccagttgttccattcagattggcctaaatcaccagctatgaacatagattttgtacgtttaagtgaggctataataactagtctccatgaatttggatttttggagcaggaacagatctcagaagctctgaacacagtctacgctatcaggaatgcatctgatcttttctcagccctttctgaaccacaaaaacaagaagttgataaaattttgactcacatacacctaaatgtcttccaggacaaggattcagctttacttctgcaaatttattcttcattttaccgatatatttatgaattattgaatattcagagtagaggctcttcgttgactttccttacacaaatctcaaaacacattttggatatcataaaacaatttaatttccaaaacatcagtaaagcatttgcatttttatttaagacagcagaggttcttgggggaatttctaatgtatcttactgtcagcaattgctttcaatttttaactttttggagcttcaggcccaatccttcatgtctacagagggccaagaactggaagtgatccacactactttgacaggcctcaaacagctgctcataattgatgaagattttcgtatttctttatttcaatatatgagccaattcttcaacagttcagtagaagacctattggataataaatgcttgatttcggacaataaacacatttcttccgtaaattattcaacaagtgaggagtcttcatttgtttttccattggcacaaattttttcaaacctctcagcaaatgtcagtgtgttcaacaagtttatgtccattcactgtaccgtttcatggcttcaaatgtggactgaaatctgggaaaccatatctcaattatttaagtttgacatgaatgttttcacatctcttcatcatggtttcactcagcttttggatgaattggaagatgatgtgaaagtctctaaaagctgccagggtatacttcccacccataatgttgctagactcatattaaatttgtttaaaaatgtaactcaagccaatgacttccataattgggaggacttcctggatctcagggattttttggtagctttaggtaatgcattagtttcagtaaaaaaacttaacttggagcaagtggagaaatcccttttcaccatggaagctgccctgcatcagttgaagacatttccattcaacgaaagtacaagcagagagtttttaaattctctgcttgaagttttcattgagtttagcagtacctcagaatatatagtcagaaatctagattcaataaatgactttctttcaaataatctcacaaattatggagaaaaatttgaaaatatcatcactgagctaagagaagcaatagtatttcttagaaatgtatcacatgatcgagatttgttttcctgtgctgatattttccaaaatgttactgagtgtattttagaagatggctttttatatgtaaatacctcacagaggatgttacgtattctagacacgttaaattccacattttcctctgagaacacaattagcagtctgaaaggatgcattgtatggttagatgtcataaaccatttgtatttgttgtctaactccagtttttcacaaggtcgtcttcaaaatattttggggaatttcagagatatagaaaacaaaatgaactctatattaaaaattgtaacttgggtgttaaatataaaaaaacctctttgttcatcaaatggctcacatataaattgtgtcaatatttacttgaaagatgtaactgactttctaaatattgtacttactacagtctttgaaaaagagaagaaacctaaatttgagattttattagctcttttaaatgattccacaaagcaagtaaggatgagtatcaacaacttaacaacagactttgattttgcatctcagtccaattggagatattttactgaattaattctaagaccaatagaaatgtcagatgaaattcctaatcagtttcaaaatatttggcttcatttaataacactggggaaggaatttcagaagcttgtaaaaggtatttattttaacatcctggaaaataattcctcttctaaaactgaaaacttgttaaacatatttgccaccagtccaaaagaaaaggatgtaaacagtgtaggcaattccatttatcacttagctagttaccttgccttcagcttatctcatgacctccaaaattcaccaaaaataataatttcacctgaaataatgaaagctacaggtcttggtattcaactgataagggatgtgttcaactccttaatgcctgtagttcatcacactagtccacaaaatgcaggttatatgcaagctttgaagaaggtaacttctgtcatgcgtacccttaagaaagcagacatagaccttttagtggatcagcttgaacaagttagtgtaaacctaatggatttctttaagaatatcagtagtgtgggaactggcaatttagtggtcaatttgcttgttggcttgatggaaaaatttgcagacagctcacattcttggaatgttaatcatctgctgcagctctcacgcctgtttcctaaagatgttgtggatgctgtgatagatgtgtactatgtgcttcctcatgctgtaaggctcctgcagggagtacctggtaaaaacatcactgaaggcctcaaggatgtctacagcttcacactccttcatggcataaccatttcaaatatcaccaaggaagacttcgcaattgtgataaaaattcttttggatacaattgaattagtatcagataagccagatattatttcagaggctttagcttgttttcctgtggtttggtgctggaatcacacaaattctggatttcggcagaattcaaagatagacccctgcaatgtccatgggctcatgtcttcttccttttatggcaaagtggccagtatacttgatcatttccacctgtctccccaaggtgaagattcaccatgttcaaatgaaagctcccgaatggaaataactaggaaagtggtctgcataattcatgaattagtggactggaattctattcttctggagctctctgaagtcttccatgttaacatttctcttgtgaaaactgtgcagaaattttggcataagatattaccgtttgtcccaccttcaataaatcaaactagggatagcatctctgaactctgtcctagtggttccataaagcaagttgctttgcaaatcatagaaaaacttaaaaatgtcaactttacaaaagttacatcaggtgaaaatattcttgacaaactaagtagtttaaacaagatccttaacattaatgaagacacagagacatctgttcaaaatattatttcctcaaatttggaaaggacagtacaattgatttctgaagactggagcctagaaaaaagtacgcataatctactctctttattcatgatgctccagaatgcaaatgtcacaggtagcagtttagaagcattatcaagttttattgaaaaaagtgaaacaccttacaactttgaagaactatggcccaagtttcaacaaatcatgaaagacctaacccaagattttagaatcagacacctgctttctgaaatgaacaaaggaatcaaaagtataaattcaatggctcttcaaaagataactttgcagtttgcccatttcctggaaatcctggattcaccgtcattgaagacattagaaattattgaagattttctattggtcacaaaaaactggcttcaggaatatgcaaatgaggattactccagaatgatagaaacattattcattcctgtgaccaatgagagttcaactgaagatatagctttgttagccaaagctattgctactttttggggctctttaaaaaatatatctagagcaggcaattttgatgttgcctttcttacccatctgctaaatcaagaacagctgactaatttctcagttgttcagctgctttttgaaaacatcctaattaatttgatcaataacttagctgggaattctcaggaagcagcttggaacttaaatgatactgaccttcaaataatgaatttcattaaccttatcttgaaccatatgcagtcagaaactagtaggaaaacagttctctctctgagaagcatagtagatttcacagaacagtttttgaaaacattcttctccctttttctaaaggaagattctgagaacaaaatatctcttctgctgaaatatttccacaaagatgttattgcagagatgagttttgtcccaaaagataaaattctagaaattctgaaactggatcaatttcttaccctgatgatacaagacagattgatgaacattttttcaagtttaaaggagactatatatcacctaatgaaaagttcatttatattagacaatggagaattttattttgatactcatcaaggactgaagttcatgcaagatttatttaatgcccttctcagggaaacttcaatgaaaaataagactgaaaataatatagactttttcacagtggtgagtcagttgtttttccatgtgaataagtctgaggacctcttcaaactcaatcaagatcttgggtcagctcttcaccttgtaagagaatgttcaacagagatggcaagacttctggatacaattttacactctcctaataaggacttctatgctttgtatcctaccctccaagaagttatacttgctaatctaacggatttgcttttctttataaataattcattccctctaagaaacagagcaacattagaaattactaagagattagttggtgctatttcaagagcaagtgaagaaagtcacgtcctgaaacccctcttagaaatgtctgggactctggtcatgctgttgaatgacagtgctgacctgagagatcttgccacatcaatggactccattgtgaaacttcttaagctggtcaagaaagtttcggggaagatgtccacagtttttaaaactcattttatctccaataccaaggacagtgtgaaattctttgacactctgtattccatcatgcaacaaagtgttcaaaatcttgtgaaagaaatagctactttaaaaaaaatagatcatttcacatttgaaaagataaatgatttgttggtgccatttcttgacttggcctttgaaatgattggggtagaaccttatatatcatcaaactctgatattttcagtatgtcacctagcatactctcatatatgaaccaatctaaggacttttctgatattttggaagaaattgctgaatttttaacatctgtgaaaatgaacttggaagatatgaggagtcttgcggtagcatttaacaatgagactcaaacattttctatggattctgtcaacttaygggaagaaattctgggttgcttagttcctataaataacatcaccaaccaaatggacttcttataccctaatccaatttccactcatagtggccctcaagatataaaatgggaaataattcatgaagtgatcctttttttggataaaatattatcacaaaacagcacagaaataggatctttcttgaaaatggtgatctgtctcaccttagaagctctttggaaaaacttaaagaaagataattggaatgtttctaatgtgttgatgacgtttactcagcatccaaataaccttttgaaaaccatagaaacagttttagaggcctccagtggaattaaaagtgactatgaaggtgatttgaataaaagtttatattttgacacacctttgagtcagaatataactcatcatcaacttgaaaaagcaatccataatgttttaagtagaatagctctctggaggaaaggacttcgttttaacaactctgaatggataacttccacaagaactttgtttcagccactttttgagattttcattaaagcaaccaccggaaagaatgtcacatcagaaaaagaagagagaaccgagaaagagatgattgactttccttatagtttcaaaccatttttctgtttggagaaatacctgggaggattatttgtattgactaaatactggcaacaaatcccactaacagatcaaagtgttgttgagatttgtgaagttttccagcagactgtgaagccctcagaagccatggagatgctgcagaaagtgaagatgatggtcgtacgtgtgctcaccatcgttgcagaaaacccttcctggaccaaggacattttgtgtgctactctgagttgcaagcaaaatgggataaggcatctcattttatctgctatacaaggggtcactttggcgcaggaccacttccaggaaattgaaaagatatggtcctcgccgaatcagctaaattgtgaaagtcttagcaagaatctttctagcaccttggagagcttcaagagcagcttggaaaatgccactggccaggactgcacaagccagccgaggctggagacggtgcagcagcacttgtacatgttggccaaaagcctcgaggaaacttggtcatcagggaatcccatcatgacttttctcagcaatttcacagtaactgaggatgtaaaaataaaagatttgatgaagaatatcaccaagttgactgaggagcttcgctcttccatccaaatctcgaatgagactatccatagcattctagaagcaaatatttcccactccaaggttctcttcagtgccctcaccgtagctctgtctggaaagtgtgatcaggaaatccttcatctcctgctgacatttcccaaaggggaaaaatcttggatcgcagcggaggaactctgtagcctgccagggtcaaaagtgtattctctgattgtgttgctgagtcgaaacttggatgtgcgagctttcatttacaagactctgatgccttctgaagcaaatggcttgctcaactccttgctggatatagtttccagcctcagcgccttgcttgccaaagcccagcacgtctttgagtatcttcctgagtttcttcacacatttaaaatcactgccttgctagaaaccctggactttcaacaggtttcacaaaatgtccaggccagaagttcagcttttggttctttccagtttgtgatgaagatggtttgcaaggaccaagcatcattccttagcgattctaatatgtttattaatttgcccagagttaaggaactcttggaagatgacaaagaaaaattcaacattcctgaagattcaacaccgttttgcttgaagctttatcaggaaattctacaattgccaaatggtgctttggtgtggaccttcctaaaacccatattgcatggaaaaatactatacacaccaaacactccagaaattaacaaggtcattcaaaaggctaattacaccttttatattgtggacaaactaaaaactttatcagaaacactgctggaaatgtccagccttttccagagaagtggaagtggccagatgttcaaccagctgcaggaggccctgagaaacaaatttgtaagaaactttgtagaaaaccagttgcacattgatgtagacaaacttactgaaaaactccagacatacggagggctgctggatgagatgtttaaccatgcaggcgctggacgcttccgtttcttgggcagcatcttggtcaatctctcttcctgcgtggcactgaaccgtttccaggctctgcagtctgtcgacatcctggagactaaagcacatgaactcttgcagcagaacagcttcttggccagtatcattttcagcaattccttattcgacaagaacttcagatcagagtctgtcaaactgccaccccatgtctcatacacaatccggaccaatgtgttatacagcgtgcgaacagatgtggtaaaaaacccttcttggaagttccaccctcagaatctaccagctgatgggttcaaatataactacgtctttgccccactgcaagacatgatcgaaagagccatcattttggtgcagactgggcaggaagccctggaaccagcagcacagactcaggcggccccttacccctgccataccagcgacctattcctgaacaacgttggtttcttttttccactgataatgatgctgacgtggatggtgtctgtggccagcatggtcagaaagttggtgtatgagcaggagatacagatagaagagtatatgcggatgatgggagtgcatccagtgatccatttcctggcctggttcctggagaacatggctgtgttgaccataagcagtgctactctggccatcgttctgaaaacaagtggcatctttgcacacagcaatacctttattgttttcctctttctcttggattttgggatgtcagtcgtcatgctgagctacctcttgagtgcatttttcagccaagctaatacagcggccctttgtaccagcctggtgtacatgatcagctttctgccctacatagttctattggttctacataaccaattaagttttgttaatcagacatttctgtgccttctttcgacaaccgcctttggacaaggggtattttttattacattcctggaaggacaagagacagggattcaatggaataatatgtaccaggctctggaacaagggggcatgacatttggctgggtttgctggatgattctttttgattcaagcctttattttttgtgtggatggtacttgagcaacttgattcctggaacatttggtttacggaaaccatggtatttcccctttactgcctcatattggaagagtgtgggtttcttggtggagaaaaggcaatactttctaagttctagtctgttcttcttcaatgagaactttgacaataaagggtcatcactgcaaaacagggaaggagagcttgaaggaagtgccccgggagtcaccctggtgtctgtgaccaaggaatatgagggccacaaggctgtggtccaagacctcagcctgaccttctacagagaccaaatcaccgccctgctggggacaaacggtgccgggaaaaccactatcatatccatgttgacggggctccaccctcccacttctggaaccatcatcatcaatggcaagaacctacagacagacctgtcgagggtcagaatggagcttggtgtgtgtccgcagcaggacatcctgttggacaacctcaccgtccgggaacatttgctgctctttgcttccataaaggcgcctcagtggaccaagaaggagctgcatcagcaagtcaatcaaactcttcaggatgtggacttaactcagcatcagcacaaacagacccgagctctgtctggaggcctgaagaggaagctctcccttggcattgctttcatgggcatgtcgaggaccgtggttctggatgagcccaccagtggggtggacccttgctcccggcatagcctgtgggacattctgctcaagtaccgagaaggtcgtacgatcatcttcacaacccaccacctggatgaagccgaagcgctgagtgaccgcgtggccgtcctccagcatgggaggctcaggtgctgcggtcctcccttctgcctgaaggaggcatatggccaggggctccgcctgacactcacgaggcagccttctgttctggaggcccatgatctgaaagacatggcttgtgttacatccctgataaagatctatattccacaagcatttctcaaagacagcagtggaagtgagctgacctacaccattccaaaggacacagacaaggcctgcttgaaagggctcttccaggccctggatgagaacctgcatcagctgcacctgacgggctatgggatctcagacaccaccttagaagaggtgtttttgatgcttttgcaagattccaacaagaaatctcacattgccctggggactgagtcagagctgcagaaccacaggcctacaggacatctgtctggctactgtggctccctagcacggcccgcaactgtgcagggcgtccagctgctccgcgcacaagtggccgcgatcctggcccggaggctccgccgcacgctgcgcgccgggaagagcaccctcgccgacctgctgctgccagtcctcttcgtggccttggccatgggcttgttcatggtgagacccctggccaccgagtaccctcccctcagactcacacctggacattaccagcgggccgagacctactttttcagcagtgggggcgacaacttggacctcacccgtgtgcttctgcggaagtttagagatcaagatttgccctgtgcagatttaaacccacgccagaagaattcttcatgctggcgcacagatcccttttctcacccagaattccaggattcatgtggctgcctgaagtgtccaaatagaagtgctagtgctccctacctgaccaaccacctgggccacacactgttgaatctctcaggcttcaatatggaggagtacttgctggcaccatctgaaaaaccaaggcttggaggttggtcttttggattaaaaatccccagtgaagctggaggtgcaaatggaaacatatcaaaacccccaactctggcaaaggtgtggtataatcagaagggttttcattccctaccttcctacttaaatcatctaaacaaccttattttgtggcagcacctaccccctactgtggactggagacaatacggaataacactctacagccacccatatggaggggccttgctgaacgaggacaagatcctggagagcatccgtcagtgtggagtggccctctgcatcgtgctgggattctccatcctgtctgcatccatcggcagctctgtggtgagggacagggtgattggagccaaaaggttgcagcacataagtggccttggctacaggatgtactggttcacaaacttcctatatgacatgctcttttacttggtttccgtctgcctgtgtgttgccgttattgtcgccttccagttaacagcttttactttccgcaagaacttggcagccacggccctcctgctgtcacttttcggatatgcaactcttccatggatgtacctgatgtccagaatcttttccagttcggacgtggctttcatttcctatgtctcactaaacttcatctttggcctttgtaccatgcccataaccattatgccccggttgctagccatcatctccaaagctaagaatttacagaatatctatgatgtcctcaagtgggtctttactatttttcctcaattctgtcttggtcaaggactggtagaactctgctataatcagatcaaatatgacctgacccacaacttcggcattgattcctatgtgagtccctttgagatgaactttctgggctggatcttcgtgcaactggcctcgcagggcacagtacttctcctcttgagggttctgctacactgggaccttctgcgatggccaaggggtcattctactctccaaggcacagtcaaatcttctaaggatacagatgttgaaaaagaggaaaagagagtgtttgaaggaaggaccaatggagacattcttgtgttatacaaccttagtaaacattatcgacgctttttccagaatattattgctgtgcaagatattagtttgggcataccaaaaggagagtgctttggacttctaggggtgaatggagctgggaagagcacgactttcaaaatgctgaatggtgaagtttctctaacttcaggacatgctatcatcaggactcccatgggagacgccgtggacctgtcttctgctggcacggcaggcgtgctcattggctactgtccccagcaggatgccctggacgagcttctgactggttgggaacatctctattattactgtagcttacgcgggattccaaggcagtgcatccctgaggttgctggagacctcatcaggcgcttacacctcgaagcccacgcggacaaacctgtggccacctacagtgggggaaccaagcggaaactctctacagccctggccctggtggggaaacctgacattcttttattggatgagcccagctctgggatggatccctgctctaagcggtacctgtggcaaacaataatgaaggaggttcgggaaggctgtgctgcggtgctgacctcccacagcatggaggagtgtgaggctctttgcacaagactggccataatggttaacggcagcttcaaatgtcttggttctcctcagcacatcaaaaataggtttggtgatggttatacagtcaaagtttggctctgtaaggaagcaaatcaacattgcactgtttctgaccacttgaagctttattttccaggaattcagttcaagggacagcacctgaatttattagaatatcatgtgccaaaaagatggggatgcctagctgacttgttcaaagttatagagaacaataaaaccttcttgaatattaagcattattccattaaccaaaccactttggagcaggtatttattaattttgcttctgagcagcagcaaactctacaatctactcttgatccatccactgacagtcaccacacacatcacttgcccatctgagcactaaagaagtttccataaggaataaaaccttgtcttccattacaattaacagtcaaggataaaacaagcacgcgcacaatcaaggagctggaacacactctccaggccgtcaaattattctcttgttcattttctattttgaatctccttgttagttaataaccaccaaatggaaaggtcattctttctgcagacttttggggagctcctccaaaacatttgttctctttaccatgccagatggacaccagcttctttgtgacaaaggcatgaatgatttgacagtgtccaaactgagacattctggagctggaaagcctgtcacactagagtgtgtgtgacatgtccactctaaacatgtcacttttctgttaagaaaactgagccccctccccacaggttaaaaaactttagtaacttgtttgtatagaaaatagtaacaaggactattttctattgttgtcatctatttactagatacatgtttttaatgattttaatgtaagcttttattaatactgatgacattatatggtatgatatgaaaaaatcaccaattttttacatataaaagatacctttttaaaaaaataggttttaagagctcttttagtatacactttagcaaaattaattaaattgaactagttactctgtatcaattacagtagttctaccagaatctcccaggttataatttatgagggtagagaaataaaatgtagatgcattttctttttcttcatttggatgaataattactgttttttgttattctaagtcagtgtttttcaaagcgtagtggtccccatattagctgcattgccatcttctgggagcttgcaagaaatgtacattctcaggatccactccagacctattgaatctcaaattctggggcttaaacaagcacattccaagttaagaaccaatgacctaagggaatgtctggttacctcctagttatacaagcaaaatctgcatagtatgtagtcttttttatttatttattttttttttttgaggcggagtctcgctctgtcacccaggctggagtgcagtggcgcgatgtcggctcactgcaagctccgcctcctgggttcacgccattctcctgcctcagcctccccagcagctgggactacaggcacacatcgccacacccggctaatttttagtatttttagtagagacggggtttcaccgtgttagccaggatggtctctatctcctgaccttgtgatccgcccccctccacctcccaaagtgctgggattacaggcgtgagccaccgtgtccggccgtagtttattttaaaatatatttttaaaagctttgtaaaaattatgtcattctcagaattgttgtcttcaaagcattgtcagatgtagagtgctcagatgtggctcttaaagactatatacatctgaatttttcatcctatagttagtaagatgcataaaatcaatccactactgaaatagtttccagtcagacatttctgagttcagacatttctcaacattcttttaacaacatttttctgaatcctcaatagaaaatcacattaatcttattttaaaatttggcctttttcaacactaacgttgagtaccggtagctttgtgatcaaaggcatatacttccttatgagatttctttactaaagcaagatttcattaaatctctatttcctaaatatcattctatacaaaagatattttttaaacggtaaggattaagacaatcactgatagctttgttgtgagcaattttgattcccatgtatcacatgaattacacttccttaaataaattacagtttatggctgtatgatttattctctaattctaacatagtctagttgtcaaaaggaaatatgtaatctttttatgattgttgaatcaataaataccaatttgtgaaacatgaatgtgtttaaactgcagtgaataaatgagatgtgctttaatttaacccaaaaaaaaaaaaaaaaaaaaaaa

SEQ ID NO: 53 - protein, 931 aa, Homo sapiens
MKIQKKLTGCSRLMLLCLSLELLLEAGAGNIHYSVPEETDKGSFVGNIAKDLGLQPQELADGGVRIVSRGRMPLFALNPRSGSLITARRIDREELCAQSMPCLVSFNILVEDKMKLFPVEVEIIDINDNTPQFQLEELEFKMNEITTPGTRVSLPFGQDLDVGMNSLQSYQLSSNPHFSLDVQQGADGPQHPEMVLQSPLDREEEAVHHLILTASDGGEPVRSGTLRIYIQVVDANDNPPAFTQAQYHINVPENVPLGTQLLMVNATDPDEGANGEVTYSFHNVDHRVAQIFRLDSYTGEISNKEPLDFEEYKMYSMEVQAQDGAGLMAKVKVLIKVLDVNDNAPEVTITSVTTAVPENFPPGTIIALISVHDQDSGDNGYTTCFIPGNLPFKLEKLVDNYYRLVTERTLDRELISGYNITITAIDQGTPALSTETHISLLVTDINDNSPVFHQDSYSAYIPENNPRGASIFSVRAHDLDSNENAQITYSLIEDTIQGAPLSAYLSINSDTGVLYALRSFDYEQFRDMQLKVMARDSGDPPLSSNVSLSLFLLDQNDNAPEILYPALPTDGSTGVELAPLSAEPGYLVTKVVAVDRDSGQNAWLSYRLLKASEPGLFSVGLHTGEVRTARALLDRDALKQSLVVAVQDHGQPPLSATVTLTVAVADRISDILADLGSLEPSAKPNDSDLTLYLVVAAAAVSCVFLAFVIVLLAHRLRRWHKSRLLQASGGGLASMPGSHFVGVDGVRAFLQTYSHEVSLTADSRKSHLIFPQPNYADTLISQESCEKKGFLSAPQSLLEDKKEPFSQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 54 - DNA, 4602 bp, Homo sapiens
atgaagattcagaaaaagctgactggctgcagcaggctgatgcttctgtgtctttctctggagctgctgttggaagctggggctgggaatattcactactcagtgccggaagagacagacaaaggttccttcgtaggcaacatcgccaaggacctagggctgcaaccccaggagctggcagatggcggagtccgcatcgtctccagaggtaggatgccgcttttcgctctgaatcctagaagtggcagcttgatcaccgcgcgcaggatagaccgggaggagctctgcgctcagagcatgccgtgtctcgtgagttttaatatccttgttgaggataaaatgaagctttttcctgttgaagtagaaataattgatattaatgacaacactccccaattccagttagaggaactggagtttaaaatgaatgaaataacgactccaggtaccagagtctcattgccttttgggcaagaccttgatgtgggtatgaactcactccagagctaccaactcagctctaaccctcatttctccctggatgtgcaacagggagccgatgggcctcaacatccagagatggtgctgcagagtcccttagacagagaagaagaagctgtccaccacctcatcctcacagcttctgatgggggtgaaccagtccgttcagggaccctcagaatttacattcaggtggtggatgcaaatgacaatcctccagcatttactcaggcacaataccatataaatgtccccgaaaacgtgccgctgggtactcagctgctcatggtaaatgccactgaccctgatgagggagccaatggggaagtaacgtactcctttcacaatgtagaccacagagtggcccaaatatttcgtttagattcttacacaggagaaatatcaaataaagaaccactagatttcgaagaatacaaaatgtattcaatggaagttcaagcccaggatggtgcggggctcatggctaaagttaaggtactgatcaaagttttggatgtaaatgataatgccccagaagtgaccatcacctctgtcaccactgcagttccagaaaactttcctcctgggaccataattgctcttatcagtgtgcatgaccaggactcaggagacaatggctacaccacatgtttcattcctggaaatttaccctttaaattggaaaagttagttgataattattaccgtttagtgactgaaagaacactggacagagaacttatctctgggtacaacatcacaataacagcaatagaccaaggaactccagctctatctactgaaactcacatttcactactagtgacagatatcaatgacaactccccagtcttccatcaggactcctactctgcctacattcccgaaaacaaccccagaggagcctccatcttctctgtgagggcccacgacttggacagcaatgagaatgcacaaatcacttactccctaatagaggacactatccagggggcacccctatctgcctacctctccatcaactccgacactggggtcctgtatgcgctgcgatccttcgactatgagcagttccgggacatgcaactgaaagtgatggcgcgggacagtggggatccgcccctcagcagcaacgtgtctctcagcctattcctgctggaccagaacgacaacgcgcccgagatcctgtaccccgccctccccacagatggttctaccggcgtggagctggcgcccctctccgcagagcccggctacctggtgaccaaggtggtggcggtggacagagactcgggccagaacgcctggctgtcctaccgcctgctcaaggccagcgagccgggactcttctcggtgggtctgcacacgggcgaggtgcgcacggcgcgagccctgctggacagagacgcgctcaagcagagtctcgtggtggccgtccaggaccacggccagcccccgctctccgccactgtcacgctcaccgtggccgtggccgacaggatctccgacatcctggccgacctgggcagcctcgagccctccgccaaacccaacgattcggacctcactctgtacctggtggtggcggcggccgcggtctcctgcgtcttcctggccttcgtcatcgtgctgctggcgcacaggctgcggcgctggcacaagtcacgtctgctacaggcttcgggaggcggcttagcgagcatgcccggttcgcactttgtgggcgtggacggggttcgggctttcctgcagacctattcccacgaggtctccctcactgcggactcgcggaagagccacctgattttcccccagcccaactatgcggacacactcatcagccaggagagctgtgagaaaaagggttttctatcagcaccccagtctttacttgaagacaaaaaggaaccattttctcagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgccacactgaccaacgcagctggcaagcgggatggcaaggccccagcaggtggcaatggcaacaagaagaagtcgggcaagaaggagaagaagtaacatggaggccaggccaagagccacagggcggcctctccccaaccagcccagcttctccttacctgcacccaggcctcagagtttcagggctaacccccagaatactggtaggggccaaggccatgctccccttgggaaacagaaacaagtgcccagtcagcacctaccccttcccccccagggggttgaatatgcaaaagcagttccgctgggaacccccatccaatcaactgctgtacccatgggggtagtggggttactgtagacaccaagaaccatttgccacaccccgtttagttacagctgaactcctccatcttccaaatcaatcaggcccatccatcccatgcctccctcctccccaccccactccaacagttcctctttcccgagtaaggtggttggggtgttgaagtaccaagtaacctacaagcctcctagttctgaaaagttggaagggcatcatgacctcttggcctctcctttgattctcaatcttcccccaaagcatggtttggtgccagccccttcacctccttccagagcccaagatcaatgctcaagttttggaggacatgatcaccatccccatggtactgatgcttgctggatttagggagggcattttgctaccaagcctcttcccaacgccctggggaccagtcttctgttttgtttttcattgtttgacgtttccactgcatgccttgacttcccccacctcctcctcaaacaagagactccactgcatgttccaagacagtatggggtggtaagataaggaagggaagtgtgtggatgtggatggtgggggcatggacaaagcttgacacatcaagttatcaaggccttggaggaggctctgtatgtcctcaggggactgacaacatcctccagattccagccataaaccaataactaggctggacccttcccactacataatagggctcagcccaggcagccagctttgggctgagctaacaggaccaatggattaaactggcatttcagtccaaggaagctcgaagcaggtttaggaccaggtccccttgagaggtcagaggggcctctgtgggtgctgggtactccagaggtgccactggtggaagggtcagcggagccccagcaggaagggtgggccagccaggccattcttagtccctgggttggggaggcagggagctagggcagggaccaaatgaacagaaagtctcagcccaggatggggcttcttcaacagggcccctgccctcctgaagcctcagtccttcaccttgccaggtgccgtttctcttccgtgaaggccactgcccaggtccccagtgcgccccctagtggccatagcctggttaaagttccccagtgcctccttgtgcatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcgggctccgcgcagccgtctgtccttgatctgcccgcggcggcccgtgttgtgttttgtgctgtgtccacgcgctaaggcgaccccctcccccgtactgacttctcctataagcgcttctcttcgcatagtcacgtagctcccaccccaccctcttcctgtgtctcacgcaagttttatactctaatatttatatggctttttttcttcgacaaaaaaataataaaacgtttcttctgaaaagctg

SEQ ID NO: 55 - DNA, 3114 bp, Homo sapiens
aatcccagctcgcagcccttgctccgcgtgtactcacgggaggactcgcagacgttactgccctcttgcgtgccccggccacccccgggcggcttgtagccggtgcgcggggtggctggggctacgtgcagagctgtcgcggagccggaacagcagcggtgaagcccctcggctcggccgagaccgccgtgcccactgctcgcctcggttgccgccgctttagccgcagccgctgctgccgccgccgggggagaggcagcctattgtctttctccgcggcgaaggtgaggagctgtctcggctcggcccgcgggggagccccgggagccgcacggagatggaggaggacatctggacagtgagcaggaggcgcttcggcccatgccgaacatcccaggggacctggagagccgggaggccatggtggcatttttcaactccgctggagccaatgcccaggaggaacaaagggtgtgctgccagcccctggctcacccagtggcctcgtcccagaaaaagccagaggtagcggccccagccccagagagtgggggtgagtctgtgtttggggagacccaccgggccctgcagggggccatggagaagctgcagcgactttatggaaggagaaggtggacctgaaggagcgggtagagaaactagagcttcaattcatccacctctcaggacagacagacaccatagggagaaagtacatcagccagggggcagtgtcagagacgcagcactgggagaggaggacatcgtcaggctggcccaggaccaggaggagatgaaggtgaacctgcaggagctgcggggcaggtgttgcagcttgtgggagaccacaaggaggggcatggcaaattctgaccattgcccagaaccctgctgatgagcccactctaggagccccaatagcccaggagcttgggtgtgctgacgagcagggtgatcaccactggattgctgacagatagaggacgtgggaccgtgactatcacccctaatctgcagtggatttggctctcggcactcccaggctgggagctggatacctgccctggcagcatgactcagactgcatgacagtcacagactcgcctctgctcctgtggtccagtggccggacaccccctgggatggctcaaaggagtcaggacttggaagtggggacatcagggtagctgaaggaaatccacacacccagagcatctcggagttcagactctcagacctgaagtaggcgcccccgggactgggctaggagttggacggaatggaggatggaggacagcgagaagaaaggaagagaaatgcaaagtgtgggcagccgccaagagtgaaaatagagggaagtgtcatgcaagtgctggacagaaggcggcaggtgggacgagccccacagccccctcctcaaaaacgaccacctccaggactcagtgatccctggggggcaggctctgccagccctcggccacacgtggctccggcacccatggtcccagtgccttggatggagacggccagttctggcggccagatgtggtgctctggaatccagtcccatttccttcctggccacgcctgtccagcggcctcttcagccgcattcagcccctacttacctggggaccccggctggggcacgagagcaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgaccccgccccagcgcagtgccggccacaccccaaggcagttgccagagcttaagccccgcccccagcagcgagaacatcccagctccacaccccccccccccccgcagccagtgctccttgtcaagctccccccgtcactccaggtgggagccaccccggtgagggggtgtgccacttgcccccagggcactcctctgggcatcccgggtgggggattttggggccgtggggggcagtctttggtacctgtgttcgtcagggatgctctgacaaccaggtgtcgtccacgggcgggggcatgggcatggtgacagtggtcctgttgatgtcaccgatgatgctgagcgcctccttcagcgcgtggtgcatgtgcagcatctcgtcgtgctgctgtgcctgctctgccaactcctccatcagtgtgttctggttcccacatgagtacatattggccagcagctccgagatgatgaactccggggtctgagagtgggcaaacagggaagaaggttgggacctggtgcctgtgccgccctggctgccttgctgggcccttctgggactgtgcgctggacttggagccccttggagtatggcttttcacacgggcttctataccgcttcgactggaagatccacctccccactgccttttctcactcagatggggacaccgaggtccagaggaaaagacacctgtcaaatgtcacagatctgggaggggacttaagacttatcatgccaagaggacacctgtctactcagtttttttttggtggggcggggggcggtgatagggtctcgctctgtcaccaggctggagtacagtgatgactgctcactgcagcctccacctcctgggctcaaagtgatcctccaacgtcagcctctcgaggagctaggactacaggcacatgccaccaccaagcccagctatttttaaaatttttgtgtggagacaaggtctcactatgtggcccaggctggtctcgaactcctgggctcaagtgatcctcctgcctcggcctccaggagtgggagttggagttgatgcctggatacaggagctctgtgggtgggagtgagacaaaacacagggtcctgagctctggggaccaagcaatgtcctctggtgaaaaaaatcctggacttgctggcagaagatttgcctcttactcgccatgtgctctgaatacatttacctgccctctgggaaaaaaaaaaaaaaaaaa

SEQ ID NO: 56 - DNA, 3357 bp, Homo sapiens
aatcccagctcgcagcccttgctccgcgtgtactcacgggaggactcgcagacgttactgccctcttgcgtgccccggccacccccgggcggcttgtagccggtgcgcggggtggctggggctacgtgcagagctgtcgcggagccggaacagcagcggtgaagcccctcggctcggccgagaccgccgtgcccactgctcgcctcggttgccgccgctttagccgcagccgctgctgccgccgccgggggagaggcagcctattgtctttctccgcggcgaaggtgaggagctgtctcggctcggcccgcgggggagccccgggagccgcacggtggcatttttcaactccgctggagccaatgcccaggaggaacaaagggtgtgctgccagcccctggctcacccagtggcctcgtcccagaaaaagccagaggtagcggccccagccccagagagtgggggtgagtctgtgtttggggagacccaccgggccctgcagggggccatggagaagctgcagcgactttatggaaggagaaggtggacctgaaggagcgggtagagaaactagagcttcaattcatccacctctcaggacagacagacaccatagggagaaagtacatcagccagggggcagtgtcagagacgcagcactgggagaggaggacatcgtcaggctggcccaggaccaggaggagatgaaggtgaacctgcaggagctgcggggcaggtgttgcagcttgtgggagaccacaaggaggggcatggcaaattctgaccattgcccagaaccctgctgatgagcccactctaggagccccaatagcccaggagcttgggtgtgctgacgagcagggtgatcaccactggattgctgacagatagaggacgtgggaccgtgactatcacccctaatctgcagtggatttggctctcggcactcccaggctgggagctggatacctgccctggcagcatgactcagactgcatgacagagaacgtggccagtggagacggcacactggaaatcagagtgaatgttcttgaaagagggtcacgggtcaacaaggcccagccaaaggatgcagtagaaccattttccttagaaatctttgggagtgaagtaggcttcagccactcccatccctgccctcgcggctaccactaccccattagtttagacagggtcgggcggggaggggtgtggagaagaaatgagcttgcctgtggcccccaggctccctctgtcctagctcaggtctgggtgccattctttacactcgtgtgctcgctcacgcacacatcacacaccttgctggtcacacagtcacagactcgcctctgctcctgtggtccagtggccggacaccccctgggatggctcaaaggagtcaggacttggaagtggggacatcagggtagctgaaggaaatccacacacccagagcatctcggagttcagactctcagacctgaagtaggcgcccccgggactgggctaggagttggacggaatggaggatggaggacagcgagaagaaaggaagagaaatgcaaagtgtgggcagccgccaagagtgaaaatagagggaagtgtcatgcaagtgctggacagaaggcggcaggtgggacgagccccacagccccctcctcaaaaacgaccacctccaggactcagtgatccctggggggcaggctctgccagccctcggccacacgtggctccggcacccatggtcccagtgccttggatggagacggccagttctggcggccagatgtggtgctctggaatccagtcccatttccttcctggccacgcctgtccagcggcctcttcagccgcattcagcccctacttacctggggaccccggctggggcacgagagcaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgaccccgccccagcgcagtgccggccacaccccaaggcagttgccagagcttaagccccgcccccagcagcgagaacatcccagctccacaccccccccccccccgcagccagtgctccttgtcaagctccccccgtcactccaggtgggagccaccccggtgagggggtgtgccacttgcccccagggcactcctctgggcatcccgggtgggggattttggggccgtggggggcagtctttggtacctgtgttcgtcagggatgctctgacaaccaggtgtcgtccacgggcgggggcatgggcatggtgacagtggtcctgttgatgtcaccgatgatgctgagcgcctccttcagcgcgtggtgcatgtgcagcatctcgtcgtgctgctgtgcctgctctgccaactcctccatcagtgtgttctggttcccacatgagtacatattggccagcagctccgagatgatgaactccggggtctgagagtgggcaaacagggaagaaggttgggacctggtgcctgtgccgccctggctgccttgctgggcccttctgggactgtgcgctggacttggagccccttggagtatggcttttcacacgggcttctataccgcttcgactggaagatccacctccccactgccttttctcactcagatggggacaccgaggtccagaggaaaagacacctgtcaaatgtcacagatctgggaggggacttaagacttatcatgccaagaggacacctgtctactcagtttttttttggtggggcggggggcggtgatagggtctcgctctgtcaccaggctggagtacagtgatgactgctcactgcagcctccacctcctgggctcaaagtgatcctccaacgtcagcctctcgaggagctaggactacaggcacatgccaccaccaagcccagctatttttaaaatttttgtgtggagacaaggtctcactatgtggcccaggctggtctcgaactcctgggctcaagtgatcctcctgcctcggcctccaggagtgggagttggagttgatgcctggatacaggagctctgtgggtgggagtgagacaaaacacagggtcctgagctctggggaccaagcaatgtcctctggtgaaaaaaatcctggacttgctggcagaagatttgcctcttactcgccatgtgctctgaatacatttacctgccctctgggaaaaaaaaaaaaaaaaaa

SEQ ID NO: 57 - DNA, 2975 bp, Homo sapiens
aatcccagctcgcagcccttgctccgcgtgtactcacgggaggactcgcagacgttactgccctcttgcgtgccccggccacccccgggcggcttgtagccggtgcgcggggtggctggggctacgtgcagagctgtcgcggagccggaacagcagcggtgaagcccctcggctcggccgagaccgccgtgcccactgctcgcctcggttgccgccgctttagccgcagccgctgctgccgccgccgggggagaggcagcctattgtctttctccgcggcgaaggtggcatttttcaactccgctggagccaatgcccaggaggaacaaagggtgtgctgccagcccctggctcacccagtggcctcgtcccagaaaaagccagaggtagcggccccagccccagagagtgggggtgagtctgtgtttggggagacccaccgggccctgcagggggccatggagaagctgcagcgactttatggaaggagaaggtggacctgaaggagcgggtagagaaactagagcttcaattcatccacctctcaggacagacagacaccatagggagaaagtacatcagccagggggcagtgtcagagacgcagcactgggagaggaggacatcgtcaggctggcccaggaccaggaggagatgaaggtgaacctgcaggagctgcggggcaggtgttgcagcttgtgggagaccacaaggaggggcatggcaaattctgaccattgcccagaaccctgctgatgagcccactctaggagccccaatagcccaggagcttgggtgtgctgacgagcagggtgatcaccactggattgctgacagatagaggacgtgggaccgtgactatcacccctaatctgcagtggatttggctctcggcactcccaggctgggagctggatacctgccctggcagcatgactcagactgcatgacagtcacagactcgcctctgctcctgtggtccagtggccggacaccccctgggatggctcaaaggagtcaggacttggaagtggggacatcagggtagctgaaggaaatccacacacccagagcatctcggagttcagactctcagacctgaagtaggcgcccccgggactgggctaggagttggacggaatggaggatggaggacagcgagaagaaaggaagagaaatgcaaagtgtgggcagccgccaagagtgaaaatagagggaagtgtcatgcaagtgctggacagaaggcggcaggtgggacgagccccacagccccctcctcaaaaacgaccacctccaggactcagtgatccctggggggcaggctctgccagccctcggccacacgtggctccggcacccatggtcccagtgccttggatggagacggccagttctggcggccagatgtggtgctctggaatccagtcccatttccttcctggccacgcctgtccagcggcctcttcagccgcattcagcccctacttacctggggaccccggctggggcacgagagcaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgaccccgccccagcgcagtgccggccacaccccaaggcagttgccagagcttaagccccgcccccagcagcgagaacatcccagctccacaccccccccccccccgcagccagtgctccttgtcaagctccccccgtcactccaggtgggagccaccccggtgagggggtgtgccacttgcccccagggcactcctctgggcatcccgggtgggggattttggggccgtggggggcagtctttggtacctgtgttcgtcagggatgctctgacaaccaggtgtcgtccacgggcgggggcatgggcatggtgacagtggtcctgttgatgtcaccgatgatgctgagcgcctccttcagcgcgtggtgcatgtgcagcatctcgtcgtgctgctgtgcctgctctgccaactcctccatcagtgtgttctggttcccacatgagtacatattggccagcagctccgagatgatgaactccggggtctgagagtgggcaaacagggaagaaggttgggacctggtgcctgtgccgccctggctgccttgctgggcccttctgggactgtgcgctggacttggagccccttggagtatggcttttcacacgggcttctataccgcttcgactggaagatccacctccccactgccttttctcactcagatggggacaccgaggtccagaggaaaagacacctgtcaaatgtcacagatctgggaggggacttaagacttatcatgccaagaggacacctgtctactcagtttttttttggtggggcggggggcggtgatagggtctcgctctgtcaccaggctggagtacagtgatgactgctcactgcagcctccacctcctgggctcaaagtgatcctccaacgtcagcctctcgaggagctaggactacaggcacatgccaccaccaagcccagctatttttaaaatttttgtgtggagacaaggtctcactatgtggcccaggctggtctcgaactcctgggctcaagtgatcctcctgcctcggcctccaggagtgggagttggagttgatgcctggatacaggagctctgtgggtgggagtgagacaaaacacagggtcctgagctctggggaccaagcaatgtcctctggtgaaaaaaatcctggacttgctggcagaagatttgcctcttactcgccatgtgctctgaatacatttacctgccctctgggaaaaaaaaaaaaaaaaaa

(19) United States
(12) Patent Application Publication
(10) Pub. No.: US 2016/0326594 A1
(43) Pub. Date: Nov. 10, 2016

(54) PROSTATE CANCER GENE PROFILES AND METHODS OF USING THE SAME

(71) Applicants: The Henry M. Jackson Foundation for the Advancement of Military Medicine, Inc., Bethesda, MD (US); Genomatix, Munich (DE)

(72) Inventors: Shiv K. Srivastava, Potomac, MD (US); Albert Dobi, Rockville, MD (US); Gyorgy Petrovics, Bethesda, MD (US); Thomas Werner, Munich (DE); Martin Seifert, Fischen/Pahl (DE); Matthias Scherf, Grobenzell (DE)

(21) Appl. No.: 15/108,909

(22) PCT Filed: Dec. 29, 2014
(86) PCT No.: PCT/US14/72610

§ 371 Date: Jun. 29, 2016

Related U.S. Application Data
(60) Provisional application No. 61/921,739, filed on Dec. 30, 2013.

(51) Int. Cl. C12Q 1/68

(52) U.S. Cl. CPC C12Q 1/6886; C12Q 2600/158; C12Q 2600/118

(57) ABSTRACT

The present disclosure provides gene expression profiles that are associated with prostate cancer, including certain gene expression profiles that differentiate between subjects of African and Caucasian descent and other gene expression profiles that are common to subjects of both African and Caucasian descent. The gene expression profiles can be measured at the nucleic acid or protein level and used to stratify prostate cancer based on ethnicity or the severity or aggressiveness of prostate cancer. The gene expression profiles can also be used to identify a subject for prostate cancer treatment. Also provided are kits for diagnosing and prognosing prostate cancer and an array comprising probes for detecting the unique gene expression profiles associated with prostate cancer in subjects of African or Caucasian descent.

### CROSS REFERENCE TO RELATED APPLICATIONS

This application claims the benefit of and relies on the filing date of, U.S. provisional patent application No. 61/921,739, filed 30 Dec. 2013, the entire disclosure of which is incorporated herein by reference.

### GOVERNMENT INTEREST

This invention was made in part with Government support. The Government has certain rights in the invention.

### SEQUENCE LISTING

The instant application contains a Sequence Listing which has been submitted electronically in ASCII format and is hereby incorporated by reference in its entirety. Said ASCII copy, created on Dec. 29, 2014, is named HMJ-145-PCT_SL.txt and is 344,410 bytes in size.

### BACKGROUND

In 2013 an estimated 238,590 men will be diagnosed with carcinoma of the prostate (CaP) and an estimated 29.720 men will die from the disease [1]. This malignancy is the second leading cause of cancer-related death in men in the United States. In addition, African American (AA) men have the highest incidence and mortality from CaP compared with other races [1]. The racial disparity exists from presentation and diagnosis through treatment, survival, and quality of life [2]. Researchers have suggested that socio-economic status (SES) contributes significantly to these disparities including CaP-specific mortality [3]. As well, there is evidence that reduced access to care is associated with poor CaP outcomes, which is more prevalent among AA men than Caucasian American (CA) men [4].

However, there are populations in which AA men have similar outcomes to CA men. Sridhar and colleagues [5] published a meta-analysis in which they concluded that when SES is accounted for, there are no differences in the overall and CaP-specific survival between AA and CA men. Similarly, the military and veteran populations (systems of equal access and screening) do not observe differences in survival across race [6], and differences in pathologic stage at diagnosis narrowed by the early 2000s in a veterans' cohort [7]. Of note, both of these studies showed that AA men were more likely to have higher Gleason scores and PSA levels than CA men [6, 7].

While socio-economic factors may contribute to CaP outcomes, they do not seem to account for all variables associated with the diagnosis and disease risk. Several studies support that AA men have a higher incidence of CaP compared to CA men [1, 8, 9]. Studies also show that AA men have a significantly higher PSA at diagnosis, higher grade disease on biopsy, greater tumor volume for each stage, and a shorter PSA doubling time before radical prostatectomy [10-12]. Biological differences between prostate cancers from CA and AA men have been noted in the tumor microenvironment with regard to stress and inflammatory responses [13]. Although controversy remains over the role of biological differences, observed differences in incidence and disease aggressiveness at presentation indicate a potential role for different pathways of prostate carcinogenesis between AA and CA men.

Over the past decade, much research has focused on alterations of cancer genes and their effects in CaP [14-16]. Variations in prevalence across ethnicity and race have been noted in the TMPRSS2/ERG gene fusion that is overexpressed in CaP and is the most common known oncogene in CaP [17, 18]. Accumulating data suggest that there are differences of ERG oncogenic alterations across ethnicities [17, 19-21]. Significantly greater ERG expression in CA men compared to AA men was noted in initial papers describing ERG overexpression and ERG splice variants [17, 21]. The difference is even more pronounced between CA and AA (50% versus 16%) in patients with high Gleason grade (8-10) tumors. Thus, ERG is a major somatic gene alteration between these ethnic groups. Yet beyond TMPRSS2/ERG, little is known regarding the genetic basis for the CaP disparity between AA and CA men remains unknown [24].

Therefore, new biomarkers and therapeutic markers that are specific for distinct ethnic populations and provide more accurate diagnostic and/or prognostic potential are needed. As such, separate gene expression profiles for patients of African and Caucasian descent can be used to diagnose or prognose CaP in distinct ethnic populations and offer more informed treatment options based on these ethnic-specific gene expression signatures.

### SUMMARY

The present disclosure provides gene expression profiles that are associated with prostate cancer and methods of using the same. The gene expression profiles can be used to detect prostate cancer cells in a sample or to predict the likelihood of a patient developing prostate cancer. The gene expression profiles can also be used to evaluate the severity or stage of prostate cancer or to assess the effectiveness of a therapy or monitor the progression or regression of prostate cancer following therapy (e.g., disease-free recurrence following surgery). The gene expression profiles can be measured at either the nucleic acid or protein level. In one aspect, the gene expression profile is specific for patients of African descent. In another aspect, the gene expression profile is specific for patients of Caucasian descent.

Accordingly, one aspect is directed to a gene expression profile that is associated with prostate cancer in a patient of African descent where the gene expression profile comprises a combination of the following genes: COL10A1, HOXC4, ESPL1, MMP9, ABCA13, PCDHGA1, and AGSK1.

Another aspect is directed to a gene expression profile that is associated with prostate cancer in a patient of Caucasian descent where the gene expression profile comprises a combination of the following genes: PCA3, ALOX15, AMACR, CDH19, OR51E2/PSGR, F5, FZD8, and CLDN.

Yet another aspect is directed to a gene expression profile that represents the top differentially expressed genes in prostate cancer in both ethnic groups and includes a combination of the following genes: DLX1, NKX2-3, CRISP3, PHGR1, THBS4, AMACR, GAP43, FFAR2, GCNT1, SIM2, STX19, KLB, APOF, LOC283177, and TRPM4. In certain embodiments, the gene expression profile comprises at least DLX1 and NKX2-3. In one embodiment, the combination includes at least DLX1 and NKX2-3.

These gene profiles can be used in a method of collecting data for diagnosing or prognosing prostate cancer, the method comprising measuring the expression of a representative number of genes in one of the disclosed gene profiles, where gene expression is measured in a sample obtained from a patient. The collected gene expression data can be used to predict whether a subject has prostate cancer or will develop prostate cancer or to predict the stage or severity of prostate cancer. The collected gene expression data can be also used to inform decisions about treating or monitoring a patient. Given the identification of these unique gene expression profiles, one of skill in the art can determine which of the identified genes to include in the gene profiling analysis. A representative number of genes may include all of the genes listed in a particular profile or some lesser number, for example, three or four or more of the genes. In certain embodiments, the method further comprises detecting expression of one or more other genes associated with prostate cancer, including, but not limited to ERG, PSA, and PCA3.

Another aspect is directed to kits for use in diagnosing or prognosing prostate cancer. In one embodiment, the kit is designed for use in diagnosing or prognosing prostate cancer in a patient of African descent and comprises a plurality of probes for detecting at least one (preferably, at least three) of the following genes (or polypeptides encoded by the same): COL10A1, HOXC4, ESPL1, MMP9, ABCA13, PCDHGA1, and AGSK1. In certain embodiments, the plurality of probes is selected from a plurality of oligonucleotide probes, a plurality of antibodies, or a plurality of polypeptide probes. In other embodiments, the plurality of probes contains probes for no more than 500, 100, 50, 25, 15, 10, 9, 8, 7, 6, 5, 4, 3, or 2 genes (or polypeptides). In one embodiment, the kit further comprises a probe for detecting expression of one or more other genes associated with prostate cancer, including, but not limited to ERG. PSA, and PCA3.

In another embodiment, the kit is designed for use in diagnosing or prognosing prostate cancer in a patient of Caucasian descent and comprises a plurality of probes for detecting at least one (preferably, at least three) of the following genes (or polypeptides encoded by the same): PCA3, ALOX15, AMACR, CDH19, OR51E2/PSGR, F5, FZD8, and CLDN. In certain embodiments, the plurality of probes is selected from a plurality of oligonucleotide probes, a plurality of antibodies, or a plurality of polypeptide probes. In other embodiments, the plurality of probes contains probes for no more than 500, 100, 50, 25, 15, 10, 9, 8, 7, 6, 5, 4, 3, or 2 genes (or polypeptides). In certain embodiments, the method further comprises detecting expression of one or more other genes associated with prostate cancer, including, but not limited to ERG, PSA, and PCA3.

In yet another embodiment, the kit for diagnosing or prognosing prostate cancer comprises a plurality of probes for detecting at least one (preferably, at least four) of the following genes (or polypeptides encoded by the same): DLX1, NKX2-3, CRISP3, PHGR1, THBS4, AMACR, GAP43, FFAR2, GCNT1, SIM2, STX19, KLB, APOF, LOC283177, and TRPM4. In one embodiment, the genes comprise DLX1 and/or NKX2-3. In certain embodiments, the plurality of probes is selected from a plurality of oligonucleotide probes, a plurality of antibodies, or a plurality of polypeptide probes. In other embodiments, the plurality of probes contains probes for no more than 500, 100, 50, 25, 15, 10, 9, 8, 7, 6, 5, 4, 3, or 2 genes (or polypeptides). In certain embodiments, the method further comprises detecting expression of one or more other genes associated with prostate cancer, including, but not limited to ERG. PSA, and PCA3.

In a related aspect, the disclosure provides an array for diagnosing and/or prognosing prostate cancer. In one embodiment, the array comprises (a) a substrate and (b) a plurality of probes immobilized on the substrate for detecting the expression of at least 3 of the following human genes: COL10A1, HOXC4, ESPL1, MMP9, ABCA13, PCDHGA1, and AGSK1. In another embodiment, the array comprises (a) a substrate and (b) a plurality of probes immobilized on the substrate for detecting the expression of at least 3 of the following human genes: PCA3, ALOX15, AMACR, CDH19, OR51E2/PSGR, F5, FZD8, and CLDN. In yet another embodiment, the array comprises (a) a substrate and (b) a plurality of probes immobilized on the substrate for detecting the expression of at least 4 of the following human genes: DLX1, NKX2-3, CRISP3, PHGR1, THBS4, AMACR, GAP43, FFAR2, GCNT1, SIM2, STX19, KLB, APOF, LOC283177, and TRPM4. In certain embodiments, the array further comprises probes for detecting expression of one or more other genes associated with prostate cancer, including, but not limited to ERG, PSA, and PCA3.

The probes are preferably arranged on the substrate within addressable elements to facilitate detection. Preferably, the array comprises a limited number of addressable elements so as to distinguish the array from a more comprehensive array, such as a genomic array or the like. Thus, in one embodiment, the array comprises 500 or fewer addressable elements. In another embodiment, the array comprises no more than 250, 100, 50, or 25 addressable elements. In another embodiment, no more than 1000 polynucleotide probes are immobilized on the array. In another aspect, the disclosure provides methods of using the arrays described herein to detect gene expression in a biological sample. Using these arrays to detect gene expression can also be part of a method for detecting or prognosing prostate cancer in a biological sample.

In another aspect, the disclosure provides methods of using the gene expression profiles to identify a patient in need of prostate cancer treatment. In one embodiment, the patient is of African descent and the method comprises a) testing a biological sample from the patient for the overexpression of a plurality of genes, wherein the plurality of genes is selected because the patient is of African descent and comprises at least three of the following genes: COL10A1, HOXC4, ESPL1, MMP9, ABCA13. PCDHGA1, and AGSK1; and b) identifying the patient as in need of prostate cancer treatment if one or more of the COL10A1, HOXC4, ESPL1, MMP9, ABCA13, PCDHGA1, and AGSK1 genes is overexpressed in the biological sample as compared to a control sample or a threshold value. In another embodiment, the patient is of Caucasian descent and the method comprises a) testing a biological sample from the patient for the overexpression of a plurality of genes, wherein the plurality of genes is selected because the patient is of Caucasian descent and comprises at least four of the following genes: PCA3, ALOX15, AMACR, CDH19, OR51E2/PSGR, F5, FZD8, and CLDN3; and b) identifying the patient as in need of prostate cancer treatment if one or more of the PCA3, ALOX15, AMACR, CDH19, OR51E2/PSGR, F5, FZD8, and CLDN3 genes is overexpressed in the biological sample as compared to a control sample or a threshold value. In certain embodiments, the method further comprises detecting expression of one or more other genes associated with prostate cancer, including, but not limited to ERG. PSA, and PCA3. The methods can also further comprise a step of treating the patient.

### BRIEF DESCRIPTION OF THE DRAWINGS

The accompanying drawings, which are incorporated in and constitute a part of this specification, illustrate certain embodiments, and together with the written description, serve to explain certain principles of the antibodies and methods disclosed herein.

FIG. 1 shows hierarchical clustering analysis (FICA) of 14 tumor and 4 normal samples using average-linkage method. The HCA reveals a distinct cluster of normal patients (GP-04, GP-10, GP-09, and GP-06) and another distinct cluster of AA, ERG fusion negative patients (GP-02, GP-10, and GP-04). Clustering is based on the expression levels of the genes. All the groups are color coded.

FIG. 2A is a heatmap with clustering of 14 tumor and 4 normal samples, with African-American patients on the left and CA on the right of the Heatmap. Genes presented in the Heatmap are the overlaps of over and under expressed genes (tumor vs. normal) for AA and CA patients.

FIG. 2B provides expression values (log 2) of top 3 over expressed genes in both tumor and normal samples from AA and CA patients.

FIG. 3A is a heatmap showing genes that are consistently over expressed in AA patients and simultaneously under expressed or show no change in CA patients.

FIG. 3B is a heatmap showing genes that are consistently over expressed in CA patients and simultaneously under expressed or show no change in AA patients.

FIG. 4 shows a schematic diagram of a system according to some embodiments of the invention. In particular, this figure illustrates various hardware, software, and other resources that may be used in implementations of computer system 106 according to disclosed systems and methods. In embodiments as shown, computer system 106 may include one or more processors 110 coupled to random access memory operating under control of or in conjunction with an operating system. The processor(s) 110 in embodiments may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processor(s) 110 may communicate with data store 112, such as a database stored on a hard drive or drive array, to access or store program instructions other data.

Processor(s) 110 may further communicate via a network interface 108, which in turn may communicate via the one or more networks 104, such as the Internet or other public or private networks, such that a query or other request may be received from client 102, or other device or service. Additionally, processor(s) 110 may utilize network interface 108 to send information, instructions, workflows query partial workflows, or other data to a user via the one or more networks 104. Network interface 104 may include or be communicatively coupled to one or more servers. Client 102 may be, e.g., a personal computer coupled to the internet.

Processor(s) 110 may, in general, be programmed or configured to execute control logic and control operations to implement methods disclosed herein. Processors 110 may be further communicatively coupled (i.e., coupled by way of a communication channel) to co-processors 114. Co-processors 114 can be dedicated hardware and/or firmware components configured to execute the methods disclosed herein. Thus, the methods disclosed herein can be executed by processor 110 and/or co-processors 114.

Other configurations of computer system 106, associated network connections, and other hardware, software, and service resources are possible.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

### DEFINITIONS

In order that the present invention may be more readily understood, certain terms are first defined. Additional definitions are set forth throughout the detailed description.

The term “of African descent” refers to individuals who self-identify as being of African descent, including individuals who self-identify as being African-American, and individuals determined to have genetic markers correlated with African ancestry, also called Ancestry Informative Markers (AIM), such as the AIMs identified in Judith Kidd et al., Analyses of a set of 128 ancestry informative single-nucleotide polymorphisms in a global set of 119 population samples, Investigative Genetics, (2):1, 2011, which reference is incorporated by reference in its entirety.

The term “of Caucasian descent” refers to individuals who self-identify as being of Caucasian descent, including individuals who self-identify as being Caucasian-American, and individuals determined to have genetic markers correlated with Caucasian (e.g., European, North African, or Asian (Western, Central or Southern) ancestry, also called. Ancestry Informative Markers (AIM), such as the AIMs identified in Judith Kidd et al., Analyses of a set of 128 ancestry informative single-nucleotide polymorphisms in a global set of 119 population samples, Investigative Genetics, (2):1, 2011, which reference is incorporated by reference in its entirety.

The term “antibody” refers to an immunoglobulin or antigen-binding fragment thereof, and encompasses any polypeptide comprising an antigen-binding fragment or an antigen-binding domain. The term includes but is not limited to polyclonal, monoclonal, monospecific, polyspecific, humanized, human, single-chain, chimeric, synthetic, recombinant, hybrid, mutated, grafted, and in vitro generated antibodies. Unless preceded by the word “intact”, the term “antibody” includes antibody fragments such as Fab, F(ab′)₂, Fv, scFv, Fd, dAb, and other antibody fragments that retain antigen-binding function. Unless otherwise specified, an antibody is not necessarily from any particular source, nor is it produced by any particular method.

The term “detecting” or “detection” means any of a variety of methods known in the art for determining the presence or amount of a nucleic acid or a protein. As used throughout the specification, the term “detecting” or “detection” includes either qualitative or quantitative detection.

The term “gene expression profile” refers to the expression levels of a plurality of genes in a sample. As is understood in the art, the expression level of a gene can be analyzed by measuring the expression of a nucleic acid (e.g., genomic DNA or mRNA) or a polypeptide that is encoded by the nucleic acid.

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The terms “polypeptide,” “peptide,” and “protein” are used interchangeably herein to refer to polymers of amino acids.

The term “polypeptide probe” as used herein refers to a labeled (e.g., isotopically labeled) polypeptide that can be used in a protein detection assay (e.g., mass spectrometry) to quantify a polypeptide of interest in a biological sample.

The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site.

A “variation” or “variant” refers to an allele sequence that is different from the reference at as little as a single base or for a longer interval.

The term “ERG” or “ERG gene” refers to Ets-related gene (ERG), which has been assigned the unique Hugo Gene Nomenclature Committee (HGNC) identifier code: HGNC:3446, and includes ERG gene fusion products that are prevalent in prostate cancer, including TMPRSS2-ERG fusion products. Analyzing the expression of ERG or the ERG gene includes analyzing the expression of ERG gene fusion products that are associated with prostate cancer, such as TMPRSS2-ERG.

Gene Expression Profiles in Prostate Cancer

Next generation sequencing techniques were used to identify new biomarkers and therapeutic targets for CaP. High quality genome sequence data and coverage obtained from histologically defined and precisely dissected primary CaP specimens (80-95% tumor, primary Gleason pattern 3) was compared between cohorts of 7 patients of Caucasian descent and 7 patients of African descent (28 samples total including matched controls from each patient) to evaluate the observed disparities of CaP incidence and mortality between the two ethnic groups. These data and analyses provide the first evaluation of prostate cancer genomes from CaP patients of African and Caucasian descent that have been matched for clinic-pathologic features.

The top differentially expressed genes in CaP in both ethnic groups include: DLX1, NKX2-3, CRISP3, PHGR1, THBS4, AMACR, GAP43, FFAR2, GCNT1, SIM2, STX19, KLB, APOF, LOC283177, and TRPM4. Thus, collecting expression data of at least 2, 3, 4, 5, 6, 7, 8, 9, or 10 of these genes from a biological sample provides a unique gene expression profile for use in diagnosing or prognosing prostate cancer in a subject.

Certain embodiments are directed to a method of collecting data for use in diagnosing or prognosing CaP, the method comprising detecting expression in a biological sample of at least 2, 3, 4, 5, 6, 7, 8, 9, 10, 11, 12, 13, 14, or 15 of the following genes: DLX1, NKX2-3, CRISP3, PHGR1, THBS4, AMACR, GAP43, FFAR2, GCNT1, SIM2, STX19, KLB, APOF, LOC283177, and TRPM4. The method may optionally include an additional step of obtaining the biological sample from a subject. The method may optionally include an additional step of diagnosing or prognosing CaP using the collected gene expression data. In one embodiment, overexpression of at least 2, 3, 4, 5, 6, 7, 8, 9, 10, 11, 12, 13, 14, or 15 of the following genes: DLX1, NKX2-3, CRISP3, PHGR1, THBS4, AMACR, GAP43, FFAR2, GCNT1, SIM2, STX19, KLB, APOF, LOC283177, and TRPM4, as compared to a control sample or threshold value indicates the presence of CaP in the biological sample or an increased likelihood of developing CaP. The methods of collecting data or diagnosing and/or prognosing CaP may further comprise detecting expression of other genes associated with prostate cancer, including, but not limited to ERG, PSA, and PCA3. In certain embodiments, the methods comprise detecting expression of no more than 100, 90, 80, 70, 60, 50, 40, 30, 25, 20, 15, 14, 13, 12, 11, 10, 9, 8, 7, 6, 5, 4, 3, or 2 genes.

In one embodiment, the methods comprise detecting expression of DLX1 and one or more of the other genes listed in Table 1. In another embodiment, the methods comprise detecting expression of NKX2-3 and one or more of the other genes listed in Table 1. In another embodiment, the methods comprise detecting expression of DLX1 and NKX2-3 and one or more of the other genes listed in Table 1. In another embodiment, the methods comprise detecting expression of PHGR1 and one or more of the other genes listed in Table 1. In another embodiment, the methods comprise detecting expression of THBS4 and one or more of the other genes listed in Table 1. In another embodiment, the methods comprise detecting expression of GAP43 and one or more of the other genes listed in Table 1. In another embodiment, the methods comprise detecting expression of FFAR2 and one or more of the other genes listed in Table 1. In another embodiment, the methods comprise detecting expression of GCNT1 and one or more of the other genes listed in Table 1. In another embodiment, the methods comprise detecting expression of SIM2 and one or more of the other genes listed in Table 1. In another embodiment, the methods comprise detecting expression of STX19 and one or more of the other genes listed in Table 1. In another embodiment, the methods comprise detecting expression of KLB and one or more of the other genes listed in Table 1. In another embodiment, the methods comprise detecting expression of APOF and one or more of the other genes listed in Table 1. In another embodiment, the methods comprise detecting expression of LOC283177 and one or more of the other genes listed in Table 1. In another embodiment, the methods comprise detecting expression of TRPM4 and one or more of the other genes listed in Table 1.

The nucleic acid and amino acid sequences for human DLX1, NKX2-3, CRISPS, PHGR1, THBS4, AMACR, GAP43, FFAR2, GCNT1, SIM2, STX19, KLB, APOF, LOC283177, and TRPM4 are known. The unique identifier code assigned by Hugo Gene Nomenclature Committee (HGNC) and Entrez Gene for these genes and the accession number of a representative sequence are provided in Table 1, which sequences are hereby incorporated by reference in their entirety.

TABLE 1

| | | Entrez | |
|---|---|---|---|
| Gene | HGNC ID | Gene ID | Accession No. |
| DLX1 | 2914 | 1745 | NM_178120.4, GI: 84043957 |
| NKX2-3 | 7836 | 159296 | NM_145285.2 GI: 148746210 |
| CRISP3 | 16904 | 10321 | NM_006061.2 GI:300244559 |
| PHGR1 | 37226 | 644844 | NM_001145643.1 GI:224548949 |
| THBS4 | 11788 | 7060 | NM_003248.4 GI:291167798 |
| AMACR | 451 | 23600 | NM_014324.5 GI:266456114 |
| GAP43 | 4140 | 2596 | AK091466.1 GI:21749841 |
| FFAR2 | 4501 | 2867 | NM_005306.2 GI:227430361 |
| GCNT1 | 4203 | 2650 | NM_001097634.1 GI:148277030 |
| SIM2 | 10833 | 6493 | NM_005069.3 GI:194239685 |
| STX19 | 19300 | 415117 | NM_001001850.2 GI:344313159 |
| KLB | 15527 | 152831 | NM_175737.3 GI:198041706 |
| APOF | 615 | 319 | BC026257.1 GI:20072209 |
| LOC283177 | N/A | 283177 | AK095081.1 GI:21754271 |
| TRPM4 | 17993 | 54795 | NM_017636.3 GI:304766649 |

The following genes were identified as being over expressed in prostate tumors of patients of Caucasian descent as compared to patients of African descent: PCA3. ALOX15, AMACR, CDH19, OR51E2/PSGR, F5, FZD8, and CLDN3. Thus, obtaining expression data of at least 1, 2, 3, 4, 5, 6, 7, or 8 of these genes provides a unique gene expression profile for use in diagnosing or prognosing prostate cancer in patients of Caucasian descent.

Certain embodiments are directed to a method of collecting data for use in diagnosing or prognosing CaP in a patient of Caucasian descent, the method comprising detecting expression in a biological sample of at least 1, 2, 3, 4, 5, 6, 7, or 8 of the following genes: PCA3, ALOX15, AMACR, CDH19, OR51E2/PSGR, F5, FZD8, and CLDN3, wherein the biological sample was obtained from the patient of Caucasian descent. The method may optionally include an additional step of obtaining the biological sample from the patient of Caucasian descent. The method may optionally include an additional step of diagnosing or prognosing CaP using the collected gene expression data. In methods of diagnosing or prognosing CaP, overexpression of at least 1, 2, 3, 4, 5, 6, 7, or 8 of the following genes: PCA3, ALOX15, AMACR, CDH19, OR51E2/PSGR, F5, FZD8, and CLDN3, as compared to a control sample or threshold value indicates the presence of CaP in the biological sample or an increased risk of developing CaP. The methods of collecting data or diagnosing and/or prognosing CaP may further comprise detecting expression of other genes associated with prostate cancer, including, but not limited to ERG, PSA, and PCA3. In certain embodiments, the methods comprise detecting expression of no more than 100, 90, 80, 70, 60, 50, 40, 30, 25, 20, 15, 14, 13, 12, 11, 10, 9, 8, 7, 6, 5, 4, 3, or 2 genes.

In one embodiment, the methods comprise detecting expression of ALOX15 and one or more of PCA3, AMACR, CDH19, OR51E2/PSGR, F5, FZD8, and CLDN3. In another embodiment, the methods comprise detecting expression of CDH19 and one or more of PCA3, AMACR, ALOX15, OR51E2/PSGR, F5, FZD8, and CLDN3. In another embodiment, the methods comprise detecting expression of F5 and one or more of PCA3, AMACR, ALOX15, OR51E2/PSGR, CDH19, FZD8, and CLDN3. In another embodiment, the methods comprise detecting expression of FZD8 and one or more of PCA3. AMACR, ALOX15, OR51E2/PSGR, CDH19, F5, and CLDN3. In another embodiment, the methods comprise detecting expression of CLDN3 and one or more of PCA3, AMACR, ALOX15, OR51E2/PSGR, CDH19, F5, and FZD8. In another embodiment, the methods comprise detecting expression of PCA3 and AMACR and one or more of ALOX15, CDH19, F5, FZD8, and CLDN3.

The unique identifier code assigned by HGNC and Entrez Gene for these genes that are more frequently overexpressed in patients of Caucasian descent and the accession number of a representative sequence are provided in Table 2, which sequences are hereby incorporated by reference in their entirety.

TABLE 2

| | | Entrez | |
|---|---|---|---|
| Gene | HGNC ID | Gene ID | NCBI Reference |
| PCA3 | 8637 | 50652 | AF103907.1 GI:6165973 |
| ALOX15 | 433 | 246 | NM_001140.3 GI:40316936 |
| AMACR | 451 | 23600 | NM_014324.5 GI:266456114 |
| CDH19 | 1758 | 28513 | NM_021153.3 GI:402534572 |
| OR51E2/PSGR | 15195 | 81285 | AY033942.1 GI:16943640 |
| F5 | 3542 | 2153 | NM_000130.4 GI:119395710 |
| FZD8 | 4046 | 8325 | AB043703.1 GI:13623798 |
| CLDN3 | 2045 | 1365 | NM_001306.3 GI:171541813 |

The following genes were identified as being over expressed in prostate tumors of patients of African ancestry as compared to patients of Caucasian descent: COL10A1, HOXC4, ESPL1, MMP9, ABCA13, PCDHGA1, and AGSK1. Thus, obtaining expression data of at least 1, 2, 3, 4, 5, 6, or 7 of these genes provides a unique gene expression profile for use in diagnosing or prognosing prostate cancer in patients of African descent.

Certain embodiments are directed to a method of collecting data for use in diagnosing or prognosing CaP in a patient of African descent, the method comprising detecting expression in a biological sample of at least 1, 2, 3, 4, 5, 6, or 7 of the following genes: COL10A1, HOXC4, ESPL1, MMP9, ABCA13, PCDHGA1, and AGSK1, wherein the biological sample was obtained from the patient of African descent. The method may optionally include an additional step of obtaining the biological sample from the patient of African descent. The method may optionally include an additional step of diagnosing or prognosing CaP using the collected gene expression data. In methods of diagnosing or prognosing CaP, overexpression of at least 1, 2, 3, 4, 5, 6, or 7 of the following genes: COL10A1, HOXC4, ESPL1, MMP9, ABCA13, PCDHGA1, and AGSK1, as compared to a control sample or threshold value indicates the presence of CaP in the biological sample or an increased risk of developing CaP. The methods of collecting data or diagnosing and/or prognosing CaP may further comprise detecting expression of other genes associated with prostate cancer, including, but not limited to ERG, PSA, and PCA3. In certain embodiments, the methods comprise detecting expression of no more than 100, 90, 80, 70, 60, 50, 40, 30, 25, 20, 15, 14, 13, 12, 11, 10, 9, 8, 7, 6, 5, 4, 3, or 2 genes.

In one embodiment, the methods comprise detecting expression of COL10A1 and one or more of HOXC4, ESPL1, MMP9, ABCA13, PCDHGA1, and AGSK1. In another embodiment, the methods comprise detecting expression of HOXC4 and one or more of COL10A1, ESPL1, MMP9, ABCA13, PCDHGA1, and AGSK1. In another embodiment, the methods comprise detecting expression of ESPL1 and one or more of COL10A1, HOXC4, MMP9, ABCA13, PCDHGA1, and AGSK1. In another embodiment, the methods comprise detecting expression of MMP9 and one or more of COL10A1, HOXC4, ESPL1, ABCA13, PCDHGA1, and AGSK1. In another embodiment, the methods comprise detecting expression of ABCA13 and one or more of COL10A1, HOXC4, ESPL1, MMP9, PCDHGA1, and AGSK1. In another embodiment, the methods comprise detecting expression of PCDHGA1 and one or more of COL10A1, HOXC4, ESPL1, MMP9, ABCA13, and AGSK1. In another embodiment, the methods comprise detecting expression of AGSK1 and one or more of COL10A1, HOXC4, ESPL1, MMP9, ABCA13, and PCDHGA1.

The unique identifier codes assigned by HGNC and Entrez Gene for these genes that are more frequently overexpressed in patients of African descent and the accession number of a representative sequence are provided in Table 3, which sequences are hereby incorporated by reference in their entirety.

TABLE 3

| | | Entrez | |
|---|---|---|---|
| Gene | HGNC ID | Gene ID | NCBI Reference |
| COL10A1 | 2185 | 1300 | NM_000493.3 GI:98985802 |
| HOXC4 | 5126 | 3221 | NM_014620.5 GI:546232084 |
| ESPL1 | 16856 | 9700 | NM_012291.4 GI:134276942 |
| MMP9 | 7176 | 4318 | NM_004994.2 GI:74272286 |
| ABCA13 | 14638 | 154664 | AY204751.1 GI:30089663 |
| PCDHGA1 | 8696 | 56114 | NM_018912.2 GI: 14196453 |
| AGSK1 | N/A | 80154 | NR_026811 GI:536293433 |
| | | | NR_033936.3 GI:536293365 |
| | | | NR_103496.2 GI:536293435 |

Additionally, whole genome sequence analysis of the 28 samples identified 65 gene mutations present with higher confidence in at least one of the 14 prostate tumors analyzed. The 65 gene mutations having the highest allele frequency in the prostate tumors analyzed occurred in the following genes: GLI1, IRX4, PAPPA, SPOP, TEX15, ZNF292, ANKRD11, FAT4, HECW2, KIAA1109, SHROOM3, SPOP, TTC36, ZNRF3, C17orf65. DEGS2, NEK3, KIAA0947, LSP1, NOX3, AKR1B1, ARHGAP12, ITGA4, PVRL4, RBM26, UNC3, CATSPERB, FCRL2, CACNA1E, CORO6, DMKN, EXT1, HEATR7B2, NDUFB5, GPR180, LRRC4, TPRA1, ZIM2, C12orf50, ELMO2, RBM26, SEC14L1, TNFSF11, C9orf125, CDC73, ITSN1, KCNK16, LRRC7, METTL6, MOSC1, RP11-50B3.2, STAB2, STARD13, PTPRT, RBPJ, UBA2, DIAPH3, IL18R1, LIPF, SLITRK5, TMEM132E, POT1, RB1CC1, TAOK1, and UNC5A. Of these 65 genes, only SPOP is known to have a mutation that is associated with prostate cancer. Thus, identifying one or more of these gene mutations in a sample can provide gene signatures useful for diagnosing or prognosing prostate cancer.

Certain embodiments are directed to a method of collecting data for use in diagnosing or prognosing CaP, the method comprising detecting expression in a biological sample of one or more mutations in at least 2, 3, 4, 5, 6, 7, 8, 9, 10, 15, 20, 25, 30, 35, 40, 45, or 50 of the following genes: GLI1, IRX4, PAPPA, SPOP, TEX15, ZNF292, ANKRD11, FAT4, HECW2, KIAA1109, SHROOM3, SPOP, TTC36, ZNRF3, C17orf65, DEGS2, NEK3, KIAA0947, LSP1, NOX3, AKR1B1, ARHGAP12, ITGA4, PVRL4, RBM26, UCN3, CATSPERB, FCRL2, CACNA1E, CORO6, DMKN, EXT1, HEATR7B2, NDUFB5, GPR180, LRRC4, TPRA1, ZIM2, C12orf50, ELMO2, RBM26, SEC14L1, TNFSF11, C9orf125, CDC73, ITSN1, KCNK16, LRRC7, METTL6, MOSC1, RP11-50B3.2, STAB2, STARD13, PTPRT, RBPJ, UBA2, DIAPH3, IL18R1, LIPF, SLITRK5, TMEM132E, POT1, RB1CC1, TAOK1, and UNC5A. The method may optionally include an additional step of obtaining the biological sample from a subject. The method may optionally include an additional step of diagnosing or prognosing CaP using the collected gene mutation data. In methods of diagnosing or prognosing CaP, detection of one or more mutations in at least 2, 3, 5, 6, 7, 8, 9, 10, 15, 20, 25, 30, 35, 40, 45, or 50 of the following genes: GLI1, IRX4, PAPPA, SPOP, TEX15, ZNF292, ANKRD11, FAT4, HECW2, KIAA1109, SHROOM3, SPOP, TTC36, ZNRF3, C17orf65, DEGS2, NEK3, KIAA0947, LSP1, NOX3, AKR1B1, ARHGAP12, ITGA4, PVRL4, RBM26, UCN3, CATSPERB, FCRL2, CACNA1E, CORO6, DMKN, EXT1, HEATR7B2, NDUFB5, GPR180, LRRC4, TPRA1, ZIM2, C12orf50, ELMO2, RBM26, SEC14L1, TNFSF11, C9orf125, CDC73, ITSN1, KCNK16, LRRC7, METTL6, MOSC1, RP11-50B3.2, STAB2, STARD13, PTPRT, RBPJ, UBA2, DIAPH3, IL18R1, LIPF, SLITRK5, TMEM132E, POT1, RB1CC1, TAOK1, and UNC5A indicates the presence of CaP in the biological sample or an increased risk to develop CaP. In certain embodiments, the methods comprise detecting expression of no more than 100, 90, 80, 70, 60, 50, 40, 30, 25, 20, 15, 14, 13, 12, 11, 10, 9, 8, 7, 6, 5, 4, 3, or 2 mutated genes.

The unique identifier code assigned by HGNC for these genes and their Entrez Gene ID are provided in Table 4, which sequences are hereby incorporated by reference in their entirety. In addition, Table 4 provides the frequency with which each mutation was identified in prostate tumors and a matched normal sample.

TABLE 4

| | | | HGNC | Entrez |
|---|---|---|---|---|
| Gene | Tumor Freq. | Normal Freq. | ID | Gene ID |
| GLI1 | G: 21/T: 23(52%) | G: 37/T: 0(0%) | 20500 | 79820 |
| IRX4 | G: 18/A: 19(51%) | G: 42/A: 0(0%) | 14875 | 79368 |
| PAPPA | C: 21/T: 20(48%) | C: 40/T: 0(0%) | 1392 | 777 |
| SPOP | A: 18/C: 15(45%) | A: 39/C: 0(0%) | 21356 | 84940 |
| TEX15 | C: 25/T: 21(45%) | C: 32/T: 0(0%) | 25063 | 93099 |
| ZNF292 | T: 14/A: 11(44%) | T: 40/A: 0(0%) | 3512 | 2131 |
| ANKRD11 | C: 19/A: 14(42%) | C: 41/A: 0(0%) | 26857 | 133558 |
| FAT4 | C: 26/T: 19(42%) | C: 30/T: 0(0%) | 7700 | 4711 |
| HECW2 | C: 22/T: 16(42%) | C: 42/T: 0(0%) | 28899 | 160897 |
| KIAA1109 | A: 19/G: 14(42%) | A: 47/G: 0(0%) | 15586 | 64101 |
| SHROOM3 | G: 23/A: 16(41%) | G: 39/A: 0(0%) | 30413 | 131601 |
| SPOP | G: 20/C: 14(41%) | G: 37/C: 0(0%) | 12875 | 23619 |
| TTC36 | G: 21/A: 15(41%) | G: 30/A: 0(0%) | 26665 | 160419 |
| ZNRF3 | C: 15/T: 10(40%) | C: 31/T: 0(0%) | 17233 | 63916 |
| C17orf65 | T: 19/C: 12(38%) | T: 41/C: 0(0%) | 20327 | 64062 |
| DEGS2 | C: 19/G: 12(38%) | C: 41/G: 0(0%) | 10698 | 6397 |
| NEK3 | G: 24/C: 15(38%) | G: 32/C: 0(0%) | 11926 | 8600 |
| KIAA0947 | G: 28/A: 17(37%) | G: 35/A: 0(0%) | 28180 | 84302 |
| LSP1 | G: 18/T: 11(37%) | G: 38/T: 0(0%) | 16783 | 79577 |
| NOX3 | C: 22/T: 13(37%) | C: 38/T: 0(0%) | 6183 | 6453 |
| AKR1B1 | T: 19/A: 11(36%) | T: 47/A: 0(0%) | 14464 | 83795 |
| ARHGAP12 | G: 24/A: 14(36%) | G: 40/A: 0(0%) | 18531 | 57554 |
| ITGA4 | A: 30/G: 17(36%) | A: 35/G: 0(0%) | 28343 | 131965 |
| PVRL4 | C: 26/T: 15(36%) | C: 21/T: 0(0%) | 26189 | 64757 |
| RBM26 | C: 30/G: 17(36%) | C: 61/G: 0(0%) | 15446 | 26121 |
| UCN3 | T: 24/G: 14(36%) | T: 41/G: 0(0%) | 18629 | 55576 |
| CATSPERB | T: 36/G: 20(35%) | T: 46/G: 0(0%) | 19164 | 90627 |
| FCRL2 | G: 26/A: 14(35%) | G: 32/A: 0(0%) | 9682 | 11122 |
| CACNA1E | C: 25/T: 13(34%) | C: 40/T: 0(0%) | 5724 | 3516 |
| CORO6 | T: 30/A: 16(34%) | T: 24/A: 0(0%) | 30661 | 10054 |
| DMKN | A: 23/C: 12(34%) | A: 37/C: 0(0%) | 15480 | 81624 |
| EXT1 | G: 23/A: 12(34%) | G: 31/A: 0(0%) | 5988 | 8809 |
| HEATR7B2 | C: 23/T: 12(34%) | C: 41/T: 0(0%) | 6622 | 8513 |
| NDUFB5 | A: 32/C: 17(34%) | A: 55/C: 0(0%) | 20295 | 26050 |
| GPR180 | A: 32/G: 16(33%) | A: 41/G: 0(0%) | 26991 | 124842 |
| LRRC4 | T: 14/G: 7(33%) | T: 40/G: 0(0%) | 17284 | 25913 |
| TPRA1 | A: 18/C: 9(33%) | A: 33/C: 0(0%) | 15574 | 9821 |
| ZIM2 | C: 28/T: 14(33%) | C: 30/T: 0(0%) | 29259 | 57551 |
| C12orf50 | C: 35/A: 17(32%) | C: 38/A: 0(0%) | 12567 | 90249 |
| ELMO2 | T: 35/C: 17(32%) | T: 45/C: 0(0%) | 20500 | 79820 |
| RBM26 | C: 34/T: 16(32%) | C: 58/T: 0(0%) | 14875 | 79368 |
| SEC14L1 | T: 31/G: 15(32%) | T: 35/G: 0(0%) | 1392 | 777 |
| TNFSF11 | A: 33/C: 16(32%) | A: 44/C: 0(0%) | 21356 | 84940 |
| C9orf125 | T: 26/G: 12(31%) | T: 27/G: 0(0%) | 25063 | 93099 |
| CDC73 | G: 31/T: 14(31%) | G: 44/T: 0(0%) | 3512 | 2131 |
| ITSN1 | T: 31/C: 14(31%) | T: 40/C: 0(0%) | 26857 | 133558 |
| KCNK16 | C: 24/A: 11(31%) | C: 39/A: 0(0%) | 7700 | 4711 |
| LRRC7 | C: 39/T: 18(31%) | C: 32/T: 0(0%) | 28899 | 160897 |
| METTL6 | A: 28/G: 13(31%) | A: 35/G: 0(0%) | 15586 | 64101 |
| MOSC1 | A: 20/G: 9(31%) | A: 35/G: 0(0%) | 30413 | 131601 |
| RP11-50B3.2 | G: 26/A: 12(31%) | G: 44/A: 0(0%) | 12875 | 23619 |
| STAB2 | G: 20/A: 9(31%) | G: 38/A: 0(0%) | 26665 | 160419 |
| STARD13 | C: 24/T: 11(31%) | C: 35/T: 0(0%) | 17233 | 63916 |
| PTPRT | C: 30/T: 13(30%) | C: 40/T: 0(0%) | 20327 | 64062 |
| RBPJ | C: 23/T: 9/G: | C: 30/T: 0(0%) | 10698 | 6397 |
| | 1(30%) | | | |
| UBA2 | T: 25/A: 11(30%) | T: 46/A: 0(0%) | 11926 | 8600 |
| DIAPH3 | C: 39/A: 16(29%) | C: 32/A: 0(0%) | 28180 | 84302 |
| IL18R1 | G: 34/T: 14(29%) | G: 42/T: 0(0%) | 16783 | 79577 |
| LIPF | G: 29/T: 12(29%) | G: 43/T: 0(0%) | 6183 | 6453 |
| SLITRK5 | G: 22/A: 9(29%) | G: 45/A: 0(0%) | 14464 | 83795 |
| TMEM132E | C: 34/T: 14(29%) | C: 32/T: 0(0%) | 18531 | 57554 |
| POT1 | T: 30/C: 12(28%) | T: 46/C: 0(0%) | 28343 | 131965 |
| RB1CC1 | A: 27/C: 11(28%) | A: 42/C: 0(0%) | 26189 | 64757 |
| TAOK1 | A: 25/C: 10(28%) | A: 44/C: 0(0%) | 15446 | 26121 |
| UNC5A | G: 27/A: 11(28%) | G: 38/A: 0(0%) | 18629 | 55576 |

The GLI1 mutation showed the highest allele frequency in the tumors analyzed and also shares a common pathway with SPOP, a gene with a mutation known to be associated with prostate cancer. Therefore, in one embodiment, the methods described herein include detecting the GLI1 mutation either alone or in combination with one or more of the gene mutations listed in Table 4.

The methods of collecting data or diagnosing and/or prognosing CaP may further comprise detecting expression of other genes associated with prostate cancer, including, but not limited to ERG. PSA, and PCA3.

Detecting Gene Expression

As used herein, measuring or detecting the expression of any of the foregoing genes or nucleic acids comprises measuring or detecting any nucleic acid transcript (e.g., mRNA, cDNA, or genomic DNA) corresponding to the gene of interest or the protein encoded thereby. If a gene is associated with more than one mRNA transcript or isoform, the expression of the gene can be measured or detected by measuring or detecting one or more of the mRNA transcripts of the gene, or all of the mRNA transcripts associated with the gene.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, microarray analysis, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual, 4^{th} Ed., Cold Spring Harbor Press, Cold Spring Harbor, N.Y., 2012. Other techniques include digital, multiplexed analysis of gene expression, such as the nCounter® (NanoString Technologies, Seattle, Wash.) gene expression assays, which are further described in [22], [23], US20100112710 and US20100047924, all of which are hereby incorporated by reference in their entirety.

Detecting a nucleic acid of interest generally involves hybridization between a target (e.g. mRNA, cDNA, or genomic DNA) and a probe. Sequences of the genes used in the prostate cancer gene expression profile are known (see above). Therefore, one of skill in the art can readily design hybridization probes for detecting those genes. See e.g., Sambrook et al., Molecular Cloning: A Laboratory Manual, 4^{th} Ed., Cold Spring Harbor Press, Cold Spring Harbor, N.Y., 2012. Each probe should be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, e.g. hybridization to the polyA tail would not provide specificity. If a target has multiple splice variants, it is possible to design a hybridization reagent that recognizes a region common to each variant and/or to use more than one reagent, each of which may recognize one or more variants.

Preferably, microarray analysis or a PCR-based method is used. In this respect, measuring the expression of the foregoing nucleic acids in prostate cancer tissue can comprise, for instance, contacting a sample containing or suspected of containing prostate cancer cells with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4^{th} Ed., Cold Spring Harbor Press, Cold Spring Harbor, N.Y., 2012. Similarly, detailed protocols for preparing and using microarrays to analyze gene expression are known in the art and described herein.

Alternatively or additionally, expression levels of genes can be determined at the protein level, meaning that levels of proteins encoded by the genes discussed above are measured. Several methods and devices are well known for determining levels of proteins including immunoassays such as described in e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays typically include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO/2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g. PCR) and monitoring the amplified nucleic acid product.

Although immunoassays have typically been used for the identification and quantification of proteins, recent advances in mass spectrometry (MS) techniques have led to the development of sensitive, high throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include two-dimensional electrophoresis, liquid chromatography, and capillary electrophoresis [25], which reference is hereby incorporated by reference in its entirety. Selected reaction monitoring (SRM), also known as multiple reaction monitoring (MRM), has also emerged as a useful high throughput MS-based technique for quantifying targeted proteins in complex biological samples, including prostate cancer biomarkers that are encoded by gene fusions (e.g., TMPRSS2/ERG) [26, 27], which references are hereby incorporated by reference in their entirety.

Samples

The methods described in this application involve analysis of gene expression profiles in prostate cells. These prostate cells are found in a biological sample, such as prostate tissue, blood, serum, plasma, urine, saliva, or prostatic fluid. Nucleic acids or polypeptides may be isolated from the cells prior to detecting gene expression.

In one embodiment, the biological sample comprises prostate tissue and is obtained through a biopsy, such as a transrectal or transperineal biopsy. In another embodiment, the biological sample is urine. Urine samples may be collected following a digital rectal examination (DRE) or a prostate biopsy. In another embodiment, the sample is blood, serum, or plasma, and contains circulating tumor cells that have detached from a primary tumor. The sample may also contain tumor-derived exosomes. Exosomes are small (typically 30 to 100 nm) membrane-bound particles that are released from normal, diseased, and neoplastic cells and are present in blood and other bodily fluids. The methods disclosed in this application can be used with samples collected from a variety of mammals, but preferably with samples obtained from a human subject.

Controls

The control can be any suitable reference that allows evaluation of the expression level of the genes in the prostate cancer cells as compared to the expression of the same genes in a sample comprising non-cancerous prostate cells, such as normal prostate epithelial cells from a matched subject, or a pool of such samples. Thus, for instance, the control can be a sample from the same subject that is analyzed simultaneously or sequentially with the test sample, or the control can be the average expression level of the genes of interest, as described above, in a pool of prostate samples known to be non-cancerous. Alternatively, the control can be defined by mRNA copy numbers of other genes in the sample, such as housekeeping genes (e.g., PBGD or GAPDH) that can be used to normalize gene expression levels. Thus, the control can be embodied, for example, in a pre-prepared microarray used as a standard or reference, or in data that reflects the expression profile of relevant genes in a sample or pool of non-cancerous samples, such as might be part of an electronic database or computer program.

Over expression and decreased expression of a gene can be determined by any suitable method, such as by comparing the expression of the genes in a test sample with a control (e.g., a positive or negative control), or by using a predetermined “cut-off” or threshold value of absolute expression. A control can be provided as previously discussed. Regardless of the method used, over expression and decreased expression can be defined as any level of expression greater than or less than the level of expression of the same genes, or other genes (e.g., housekeeping genes), in non-cancerous prostate cells or tissue. By way of further illustration, over expression can be defined as expression that is at least about 1.2-fold, 1.5-fold, 2-fold, 2.5-fold, 5-fold, 10-fold, 20-fold, 50-fold, 100-fold higher or even greater expression as compared to non-cancerous prostate cells or tissue, and decreased expression can similarly be defined as expression that is at least about 1.2-fold, 1.5-fold, 2-fold, 2.5-fold, 5-fold, 10-fold, 20-fold, 50-fold, 100-fold lower or even lower expression as compared to non-cancerous prostate cells or tissue. In one embodiment, over expression or decreased expression as used herein is defined as expression that is at least about 2-fold higher or lower, respectively, as compared to a control sample or threshold value.

Prostate Cancer

This disclosure provides gene expression profiles that are associated with prostate cancer. The gene expression profiles can be used to detect prostate cancer cells in a sample or to measure the severity or aggressiveness of the prostate cancer, for example, distinguishing between well differentiated prostate (WD) cancer and poorly differentiated (PD) prostate cancer.

When prostate cancer is found in a biopsy, it is typically graded to estimate how quickly it is likely to grow and spread. The most commonly used prostate cancer grading system, called Gleason grading, evaluates prostate cancer cells on a scale of 1 to 5, based on their pattern when viewed under a microscope.

Cancer cells that still resemble healthy prostate cells have uniform patterns with well-defined boundaries and are considered well differentiated (Gleason grades 1 and 2). The more closely the cancer cells resemble prostate tissue, the more the cells will behave like normal prostate tissue and the less aggressive the cancer. Gleason grade 3, the most common grade, shows cells that are moderately differentiated, that is, still somewhat well-differentiated, but with boundaries that are not as well-defined. Poorly-differentiated cancer cells have random patterns with poorly defined boundaries and no longer resemble prostate tissue (Gleason grades 4 and 5), indicating a more aggressive cancer.

Prostate cancers often have areas with different grades. A combined Gleason score is determined by adding the grades from the two most common cancer cell patterns within the tumor. For example, if the most common pattern is grade 4 and the second most common pattern is grade 3, then the combined Gleason score is 4+3=7. If there is only one pattern within the tumor, the combined. Gleason score can be as low as 1+1=2 or as high as 5+5=10. Combined scores of 2 to 4 are considered well-differentiated, scores of 5 to 6 are considered moderately-differentiated and scores of 7 to 10 are considered poorly-differentiated. Cancers with a high Gleason score are more likely to have already spread beyond the prostate gland at the time they were found.

In general, the lower the Gleason score, the less aggressive the cancer and the better the prognosis (outlook for cure or long-term survival). The higher the Gleason score, the more aggressive the cancer and the poorer the prognosis for long-term, metastasis-free survival.

Array

A convenient way of measuring RNA transcript levels for multiple genes in parallel is to use an array (also referred to as microarrays in the art). Techniques for using arrays to assess and compare gene expression levels are well known in the art and include appropriate hybridization, detection and data processing protocols. A useful array includes multiple polynucleotide probes (typically DNA) that are immobilized on a solid substrate (e.g. a glass support such as a microscope slide, or a membrane) in separate locations (e.g., addressable elements) such that detectable hybridization can occur between the probes and the transcripts to indicate the amount of each transcript that is present. The arrays disclosed in this application can be used in methods of detecting the expression of a desired combination of genes, which combinations are discussed throughout this application.

In one embodiment, the array comprises (a) a substrate and (b) 2, 3, 4, 5, 6, 7, 8, 9, or 10 or more different addressable elements that each comprise at least one polynucleotide probe for detecting the expression of an mRNA transcript (or cDNA synthesized from the mRNA transcript) of one of the following human genes: DLX1, NKX2-3, CRISP3, PHGR1, THBS4, AMACR, GAP43, FFAR2, GCNT1, SIM2, STX19, KLB, APOF, LOC283177, and TRPM4.

In another embodiment, the array comprises (a) a substrate and (b) 2, 3, 4, 6, 7, or 8 or more different addressable elements that each comprise at least one polynucleotide probe for detecting the expression of an mRNA transcript (or cDNA synthesized from the mRNA transcript) of one of the following human genes: PCA3, ALOX15, AMACR, CDH19, OR51E2/PSGR, F5, FZD8, and CLDN3.

In yet another embodiment, the array comprises (a) a substrate and (b) 2, 3, 4, 5, 6, or 7 or more different addressable elements that each comprise at least one polynucleotide probe for detecting the expression of an mRNA transcript (or cDNA synthesized from the mRNA transcript) of one of the following human genes: COL10A1, HOXC4, ESPL1, MMP9, ABCA13, PCDHGA1, and AGSK1.

As used herein, the term “addressable element” means an element that is attached to the substrate at a predetermined position and specifically binds a known target molecule, such that when target-binding is detected (e.g., by fluorescent labeling), information regarding the identity of the bound molecule is provided on the basis of the location of the element on the substrate. Addressable elements are “different” for the purposes of the present disclosure if they do not bind to the same target gene. The addressable element comprises one or more polynucleotide probes specific for an snRNA transcript of a given gene, or a cDNA synthesized from the mRNA transcript. The addressable element can comprise more than one copy of a polynucleotide, can comprise more than one different polynucleotide, provided that all of the polynucleotides bind the same target molecule. Where a gene is known to express more than one mRNA transcript, the addressable element for the gene can comprise different probes for different transcripts, or probes designed to detect a nucleic acid sequence common to two or more (or all) of the transcripts. Alternatively, the array can comprise an addressable element for the different transcripts. The addressable element also can comprise a detectable label, suitable examples of which are well known in the art.

The array can comprise addressable elements that bind to mRNA or cDNA other than that of 1) DLX1, NKX2-3, CRISPS, PHGR1, THBS4, AMACR, GAP43, FFAR2, GCNT1, SIM2, STX19, KLB, APOF, LOC283177, and TRPM4; 2) PCA3, ALOX15, AMACR, CDH19, OR51E2/PSGR, F5, FZD8, and CLDN3; or 3) COL10A1, HOXC4, ESPL1, MMP9, ABCA13, PCDHGA1, and AGSK1. However, an array capable of detecting a vast number of targets (e.g., mRNA or polypeptide targets), such as arrays designed for comprehensive expression profiling of a cell line, chromosome, genome, or the like, are not economical or convenient for collecting data to use in diagnosing an/or prognosing prostate cancer. Thus, to facilitate the convenient use of the array as a diagnostic tool or screen, for example, in conjunction with the methods described herein, the array preferably comprises a limited number of addressable elements. In this regard, in one embodiment, the array comprises no more than about 1000 different addressable elements, more preferably no more than about 500 different addressable elements, no more than about 250 different addressable elements, or even no more than about 100 different addressable elements, such as about 75 or fewer different addressable elements, or even about 50 or fewer different addressable elements. Of course, even smaller arrays can comprise about 25 or fewer different addressable elements, such as about 15 or fewer different addressable elements or about 12 or fewer different addressable elements. The array can even be limited to about 7 different addressable elements without interfering with its functionality.

It is also possible to distinguish these diagnostic arrays from the more comprehensive genomic arrays and the like by limiting the number of polynucleotide probes on the array. Thus, in one embodiment, the array has polynucleotide probes for no more than 1000 genes immobilized on the substrate. In other embodiments, the array has oligonucleotide probes for no more than 500, 250, 100, 50, 25, 15, 10, 9, 8, 7, 6, 5, 4, 3, or 2 genes immobilized on the substrate.

The substrate can be any rigid or semi-rigid support to which polynucleotides can be covalently or non-covalently attached. Suitable substrates include membranes, filters, chips, slides, wafers, fibers, beads, gels, capillaries, plates, polymers, microparticles, and the like. Materials that are suitable for substrates include, for example, nylon, glass, ceramic, plastic, silica, aluminosilicates, borosilicates, metal oxides such as alumina and nickel oxide, various clays, nitrocellulose, and the like.

The polynucleotides of the addressable elements (also referred to as “probes”) can be attached to the substrate in a pre-determined 1- or 2-dimensional arrangement, such that the pattern of hybridization or binding to a probe is easily correlated with the expression of a particular gene. Because the probes are located at specified locations on the substrate (i.e., the elements are “addressable”), the hybridization or binding patterns and intensities create a unique expression profile, which can be interpreted in terms of expression levels of particular genes and can be correlated with prostate cancer in accordance with the methods described herein.

Polynucleotide and polypeptide probes can be generated by any suitable method known in the art (see e.g., Sambrook et al., Molecular Cloning: A Laboratory Manual, 4^{th} Ed., Cold Spring Harbor Press, Cold Spring Harbor, N.Y., 2012). For example, polynucleotide probes that specifically bind to the mRNA transcripts of the genes described herein (or cDNA synthesized therefrom) can be created using the nucleic acid sequences of the mRNA or cDNA targets themselves (e.g., nucleic acid sequences disclosed in Tables 1-4) by routine techniques (e.g., PCR or synthesis). As used herein, the term “fragment” means a contiguous part or portion of a polynucleotide sequence comprising about 10 or more nucleotides, about 15 or more nucleotides, about 20 or more nucleotides, about 30 or more, or even about 50 or more nucleotides. By way of further illustration, a polynucleotide probe that binds to an mRNA transcript of DLX1 (or cDNA corresponding thereto) can be provided by a polynucleotide comprising a nucleic acid sequence that is complementary to the mRNA transcript (e.g., SEQ ID NO: 2) or a fragment thereof, or sufficiently complementary to SEQ ID NO: 2 or fragment thereof that it selectively binds to SEQ ID NO: 2. The same is true with respect to the other genes described herein. The exact nature of the polynucleotide probe is not critical to the invention; any probe that will selectively bind the mRNA or cDNA target can be used. Typically, the polynucleotide probes will comprise 10 or more nucleic acids, 20 or more, 50 or more, or 100 or more nucleic acids. In order to confer sufficient specificity, the probe will have a sequence identity to a complement of the target sequence (e.g., nucleic acid sequences disclosed in Tables 1-4) of about 90% or more, preferably about 95% or more (e.g., about 98% or more or about 99% or more) as determined, for example, using the well-known Basic Local Alignment Search Tool (BLAST) algorithm (available through the National Center for Biotechnology Information (NCBI), Bethesda, Md.).

Stringency of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium, citrate) and 50% formamide at 55° C., followed by a high-stringency wash consisting of 0.1×SSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The array can comprise other elements common to polynucleotide arrays. For instance, the array also can include one or more elements that serve as a control, standard, or reference molecule, such as a housekeeping gene or portion thereof (e.g., PBGD or GAPDH), to assist in the normalization of expression levels or the determination of nucleic acid quality and binding characteristics, reagent quality and effectiveness, hybridization success, analysis thresholds and success, etc. These other common aspects of the arrays or the addressable elements, as well as methods for constructing and using arrays, including generating, labeling, and attaching suitable probes to the substrate, consistent with the invention are well-known in the art. Other aspects of the array are as previously described herein with respect to the methods of the invention.

In one embodiment, the array comprises (a) a substrate and (b) two or more different addressable elements that each comprise at least one polynucleotide probe for detecting the expression of an mRNA transcript of one of the following human genes: DLX1, NKX2-3, CRISP3, PHGR1, THBS4, AMACR, GAP43, FFAR2, GCNT1, SIM2, STX19, KLB, APOF, LOC283177, and TRPM4, wherein the array comprises no more than 500, 250, 100, 50, 25, 15, 10, 9, 8, 7, 6, 5, 4, 3, or 2 addressable elements. In certain embodiments, the array comprises at least 3, 4, 5, 6, 7, 10, 12, or 15 different addressable elements.

In another embodiment, the array comprises two or more different addressable elements each of which comprises at least one polynucleotide probe for detecting the expression of an mRNA transcript of one of the following human genes: PCA3, ALOX15, AMACR, CDH19, OR51E2/PSGR, F5, FZD8, and CLDN3, wherein the array comprises no more than 500, no more than 250, no more than 100, no more than 50, no more than 25, or no more than 15 addressable elements. In one embodiment, the array comprises at least 3, 4, 5, 6, 7, 10, 12, or 15 different addressable elements.

In another embodiment, the array comprises two or more different addressable elements each of which comprises at least one polynucleotide probe for detecting the expression of an mRNA transcript of one of the following human genes: COL10A1, HOXC4, ESPL1, MMP9, ABCA13, PCDHGA1, and AGSK1, wherein the array comprises no more than 500, 250, 100, 50, 25, 15, 10, 9, 8, 7, 6, 5, 4, 3, or 2 addressable elements. In one embodiment, the array comprises at least 3, 4, 5, 6, 7, 10, 12, or 15 different addressable elements.

An array can also be used to measure protein levels of multiple proteins in parallel. Such an array comprises one or more supports bearing a plurality of ligands that specifically bind to a plurality of proteins, wherein the plurality of proteins comprises no more than 500, 250, 100, 50, 25, 15, 10, 9, 8, 7, 6, 5, 4, 3, or 2 different proteins. The ligands are optionally attached to a planar support or beads. In one embodiment, the ligands are antibodies. The proteins that are to be detected using the array correspond to the proteins encoded by the nucleic acids of interest, as described above, including the specific gene expression profiles disclosed. Thus, each ligand (e.g. antibody) is designed to bind to one of the target proteins (e.g., polypeptide sequences disclosed in Tables 1-4). As with the nucleic acid arrays, each ligand is preferably associated with a different addressable element to facilitate detection of the different proteins in a sample.

Patient Treatment

This application describes methods of diagnosing and prognosing prostate cancer in a sample obtained from a subject, in which gene expression in prostate cells and/or tissues are analyzed. If a sample shows over expression of certain genes or the expression of certain gene mutations, then there is an increased likelihood that the subject has prostate cancer or a less or more advanced stage (e.g., WD or PD prostate cancer) of prostate cancer. In the event of such a result, the methods of detecting or prognosing prostate cancer may include one or more of the following steps: informing the patient that they are likely to have prostate cancer, WD prostate cancer or PD prostate cancer; confirmatory histological examination of prostate tissue; and/or treating the patient by a prostate cancer therapy.

Thus, in certain aspects, if the detection step indicates that the subject has prostate cancer, the methods further comprise a step of taking a prostate biopsy from the subject and examining the prostate tissue in the biopsy (e.g., histological examination) to confirm whether the patient has prostate cancer. Alternatively, the methods of detecting or prognosing prostate cancer may be used to assess the need for therapy or to monitor a response to a therapy (e.g., disease-free recurrence following surgery or other therapy), and, thus may include an additional step of treating a subject having prostate cancer.

Prostate cancer treatment options include surgery, radiation therapy, hormone therapy, chemotherapy, biological therapy, or high intensity focused ultrasound. Drugs approved for prostate cancer include: Enzalutamide (XTANDI), Abiraterone Acetate, Cabazitaxel, Degarelix, Jevtana (Cabazitaxel), Prednisone, Provenge (Sipuleucel-T), Sipuleucel-T, or Docetaxel. Thus a method as described in this application may, after a positive result, include a further step of surgery, radiation therapy, hormone therapy, chemotherapy, biological therapy, or high intensity focused ultrasound.

Drug Screening

The gene expression profiles associated with prostate cancer or lack thereof provided by the methods described in this application can also be useful in screening drugs, either in clinical trials or in animal models of prostate cancer. A clinical trial can be performed on a drug in similar fashion to the monitoring of an individual patient, except that the drug is administered in parallel to a population of prostate cancer patients, usually in comparison with a control population administered a placebo.

The changes in expression levels of genes can be analyzed in individual patients and across a treated or control population. Analysis at the level of an individual patient provides an indication of the overall status of the patient at the end of the trial (i.e., whether gene expression profile indicates the presence or severity (e.g., WD or PD) of prostate cancer) and/or an indication whether that profile has changed toward or away from such indication in the course of the trial. Results for individual patients can be aggregated for a population allowing comparison between treated and control population.

Similar trials can be performed in non-human animal models of prostate cancer. In this case, the expression levels of genes detected are the species variants or homologs of the human genes referenced above in whatever species of non-human animal on which tests are being conducted. Although the average expression levels of human genes determined in human prostate cancer patients are not necessarily directly comparable to those of homolog genes in an animal model, the human values can nevertheless be used to provide an indication whether a change in expression level of a non-human homolog is in a direction toward or away from the diagnosis of prostate cancer or prognosis of WD or PD prostate cancer. The expression profile of individual animals in a trial can provide an indication of the status of the animal at the end of the trial (i.e., whether gene expression profile indicates the presence or severity (e.g., WD or PD) of prostate cancer) and/or change in such status during the trial. Results from individual animals can be aggregated across a population and treated and control populations compared. Average changes in the expression levels of genes can then be compared between the two populations.

Computer Implemented Models

In accordance with all aspects and embodiments of the invention, the methods provided may be computer-implemented.

Gene expression levels can be analyzed and associated with status of a subject (e.g., presence of prostate cancer or severity of disease (e.g., WD or PD prostate cancer)) in a digital computer. Optionally, such a computer is directly linked to a scanner or the like receiving experimentally determined signals related to gene expression levels. Alternatively, expression levels can be input by other means. The computer can be programmed to convert raw signals into expression levels (absolute or relative), compare measured expression levels with one or more reference expression levels, or a scale of such values. The computer can also be programmed to assign values or other designations to expression levels based on the comparison with one or more reference expression levels, and to aggregate such values or designations for multiple genes in an expression profile. The computer can also be programmed to output a value or other designation providing an indication of the presence or severity of prostate cancer as well as any of the raw or intermediate data used in determining such a value or designation.

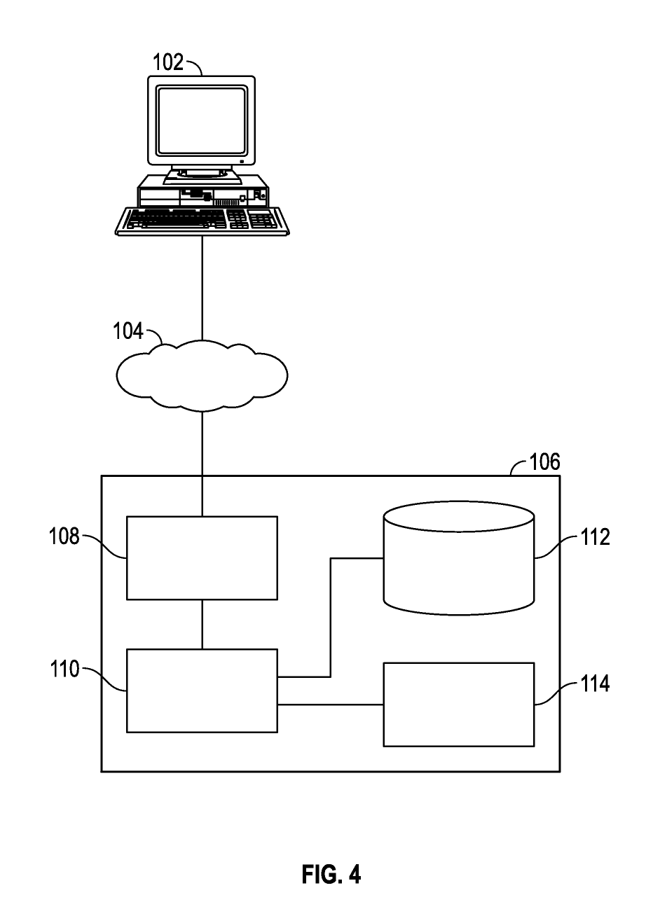

A typical computer (see U.S. Pat. No. 6,785,613; FIGS. 4 and 5) includes a bus which interconnects major subsystems such as a central processor, a system memory, an input/output controller, an external device such as a printer via a parallel port, a display screen via a display adapter, a serial port, a keyboard, a fixed disk drive and a port (e.g., USB port) operative to receive an external memory storage device. Many other devices can be connected such as a scanner via I/O controller, a mouse connected to serial port or a network interface. The computer contains computer readable media holding codes to allow the computer to perform a variety of functions. These functions include controlling automated apparatus, receiving input and delivering output as described above. The automated apparatus can include a robotic arm for delivering reagents for determining expression levels, as well as small vessels, e.g., microtiter wells for performing the expression analysis.

A typical computer system 106 may also include one or more processors 110 coupled to random access memory operating under control of or in conjunction with an operating system as set forth in FIG. 4 and discussed above.

In one embodiment, any of the computer-implemented methods of the invention may comprise a step of obtaining by at least one processor information reflecting the expression level of at least 2, 3, 4, 5, 6, 7, 8, 9, 10, 11, 12, 13, 14, or 15 of the following human genes: DLX1, NKX2-3, CRISPS, PHGR1, THBS4, AMACR, GAP43, FFAR2, GCNT1, SIM2, STX19, KLB, APOF, LOC283177, and TRPM4 in a biological sample.

In one embodiment, the computer-implemented methods comprise obtaining by at least one processor information reflecting the expression level of DLX1 and one or more of the other genes listed in Table 1. In another embodiment, the computer-implemented methods comprise obtaining by at least one processor information reflecting the expression level of NKX2-3 and one or more of the other genes listed in Table 1. In another embodiment, the computer-implemented methods comprise obtaining by at least one processor information reflecting the expression level of DLX1 and NKX2-3 and one or more of the other genes listed in Table 1. In another embodiment, the computer-implemented methods comprise obtaining by at least one processor information reflecting the expression level of PHGR1 and one or more of the other genes listed in Table 1. In another embodiment, the computer-implemented methods comprise obtaining by at least one processor information reflecting the expression level of THBS4 and one or more of the other genes listed in Table 1. In another embodiment, the computer-implemented methods comprise obtaining by at least one processor information reflecting the expression level of GAP43 and one or more of the other genes listed in Table 1. In another embodiment, the computer-implemented methods comprise obtaining by at least one processor information reflecting the expression level of FFAR2 and one or more of the other genes listed in Table 1. In another embodiment, the computer-implemented methods comprise obtaining by at least one processor information reflecting the expression level of GCNT1 and one or more of the other genes listed in Table 1. In another embodiment, the computer-implemented methods comprise obtaining by at least one processor information reflecting the expression level of SIM2 and one or more of the other genes listed in Table 1. In another embodiment, the computer-implemented methods comprise obtaining by at least one processor information reflecting the expression level of STX19 and one or more of the other genes listed in Table 1. In another embodiment, the computer-implemented methods comprise obtaining by at least one processor information reflecting the expression level of KLB and one or more of the other genes listed in Table 1. In another embodiment, the computer-implemented methods comprise obtaining by at least one processor information reflecting the expression level of APOF and one or more of the other genes listed in Table 1. In another embodiment, the computer-implemented methods comprise obtaining by at least one processor information reflecting the expression level of LOC283177 and one or more of the other genes listed in Table 1. In another embodiment, the computer-implemented methods comprise obtaining by at least one processor information reflecting the expression level of TRPM4 and one or more of the other genes listed in Table 1.

In another embodiment, any of the computer-implemented methods of the invention may comprise a step of obtaining by at least one processor information reflecting the expression level of at least 2, 3, 4, 5, 6, 7, or 8 of the following human genes: PCA3, ALOX15, AMACR, CDH19, OR51E2/PSGR, F5. FZD8, and CLDN3 in a biological sample obtained from a patient of Caucasian descent.

In one embodiment, the computer-implemented methods comprise obtaining by at least one processor information reflecting the expression level of ALOX15 and one or more of PCA3, AMACR, CDH19, OR51E2/PSGR, F5, FZD8, and CLDN3. In another embodiment, the computer-implemented methods comprise obtaining by at least one processor information reflecting the expression level of CDH19 and one or more of PCA3, AMACR, ALOX15, OR51E2/PSGR, F5, FZD8, and CLDN3. In another embodiment, the computer-implemented methods comprise obtaining by at least one processor information reflecting the expression level of F5 and one or more of PCA3, AMACR, ALOX15, OR51E2/PSGR, CDH19, FZD8, and CLDN3. In another embodiment, the computer-implemented methods comprise obtaining by at least one processor information reflecting the expression level of FZD8 and one or more of PCA3. AMACR, ALOX15, OR51E2/PSGR, CDH19, F5, and CLDN3. In another embodiment, the computer-implemented methods comprise obtaining by at least one processor information reflecting the expression level of CLDN3 and one or more of PCA3, AMACR, ALOX15, OR51E2/PSGR, CDH19, F5, and FZD8. In another embodiment, the computer-implemented methods comprise obtaining by at least one processor information reflecting the expression level of PCA3 and AMACR and one or more of ALOX15, CDH19, F5, FZD8, and CLDN3.

In another embodiment, any of the computer-implemented methods of the invention may comprise a step of obtaining by at least one processor information reflecting the expression level of at least 2, 3, 4, 5, 6, or 7 of the following human genes: COL10A1, HOXC4, ESPL1, MMP9, ABCA13, PCDHGA1, and AGSK1 in a biological sample obtained from a patient of African descent.

In one embodiment, the computer-implemented methods comprise obtaining by at least one processor information reflecting the expression level of COL10A1 and one or more of HOXC4, ESPL1, MMP9, ABCA13, PCDHGA1, and AGSK1. In another embodiment, the computer-implemented methods comprise obtaining by at least one processor information reflecting the expression level of HOXC4 and one or more of COL10A1, ESPL1, MMP9, ABCA13, PCDHGA1, and AGSK1. In another embodiment, the computer-implemented methods comprise obtaining by at least one processor information reflecting the expression level of ESPL1 and one or more of COL10A1, HOXC4, MMP9, ABCA13, PCDHGA1, and AGSK1. In another embodiment, the computer-implemented methods comprise obtaining by at least one processor information reflecting the expression level of MMP9 and one or more of COL10A1, HOXC4, ESPL1, ABCA13, PCDHGA1, and AGSK1. In another embodiment, the computer-implemented methods comprise obtaining by at least one processor information reflecting the expression level of ABCA13 and one or more of COL10A1, HOXC4, ESPL1, MMP9, PCDHGA1, and AGSK1. In another embodiment, the computer-implemented methods comprise obtaining by at least one processor information reflecting the expression level of PCDHGA1 and one or more of COL10A1, HOXC4, ESPL1, MMP9, ABCA13, and AGSK1. In another embodiment, the computer-implemented methods comprise obtaining by at least one processor information reflecting the expression level of AGSK1 and one or more of COL10A1, HOXC4, ESPL1, MMP9. ABCA13, and PCDHGA1.

In another embodiment of the computer-implemented methods of the invention, the methods may additionally comprise the steps of i) determining by at least one processor a difference between the expression level of one or more control genes and the expression level of 1) at least 2, 3, 4, 5, 6, 7, 8, 9, 10, 11, 12, 13, 14, or 15 of the following human genes: DLX1, NKX2-3, CRISP3, PHGR1, THBS4, AMACR, GAP43, FFAR2, GCNT1, SIM2, STX19, KLB, APOF, LOC283177, and TRPM4 in a biological sample; 2) at least 2, 3, 4, 5, 6, 7, or 8 of the following human genes: PCA3, ALOX15, AMACR, CDH19, OR51E2/PSGR, F5, FZD8, and CLDN3 in a biological sample obtained from a patient of Caucasian descent; or 3) at least 2, 3, 4, 5, 6, or 7 of the following human genes: COL10A1, HOXC4, ESPL1, MMP9, ABCA13, PCDHGA1, and AGSK1 in a biological sample obtained from a patient of African descent; and (ii) outputting in user readable format the difference obtained in the determining step.

In another embodiment of the computer-implemented methods of the invention, the methods may further comprise outputting in user readable format a determination that the subject has prostate cancer, well differentiated prostate cancer, or poorly differentiated prostate cancer based on the difference obtained in the outputting step.

Kits

The polynucleotide probes and/or primers or antibodies or polypeptide probes that are used in the methods described in this application can be arranged in a kit. Thus, one embodiment is directed to a kit for diagnosing or prognosing prostate cancer comprising a plurality of polynucleotide probes for detecting at least 2, 3, 4, 5, 6, 7, 8, 9, 10, 11, 12, 13, 14, or 15 of the following human genes: DLX1, NKX2-3, CRISP3, PHGR1, THBS4, AMACR, GAP43, FFAR2, GCNT1, SIM2, STX19, KLB, APOF, LOC283177, and TRPM4, wherein the plurality of polynucleotide probes contains polynucleotide probes for no more than 500, 250, 100, 50, 25, 15, 10, 9, 8, 7, 6, 5, 4, 3, or 2 genes. In one embodiment, the plurality of polynucleotide probes comprises polynucleotide probes for detecting at least 4 or 5 of the aforementioned genes, wherein the plurality of polynucleotide probes contains polynucleotide probes for no more than 10 genes. The polynucleotide probes may be optionally labeled. The kit may optionally include polynucleotide primers for amplifying a portion of the mRNA transcripts from at least 2, 3, 4, 5, 6, 7, 8, 9, 10, 11, 12, 13, 14, or 15 of the following human genes: DLX1, NKX2-3, CRISP3, PHGR1, THBS4, AMACR, GAP43, FFAR2, GCNT1, SIM2, STX19, KLB, APOF, LOC283177, and TRPM4.

Another embodiment is directed to a kit for diagnosing or prognosing prostate cancer in a patient of Caucasian descent, the kit comprising a plurality of polynucleotide probes for detecting at least 3, 4, 5, 6, 7, or 8 of the following human genes: PCA3, ALOX15, AMACR, CDH19, OR51E2/PSGR, F5, FZD8, and CLDN3, wherein the plurality of polynucleotide probes contains polynucleotide probes for no more than 500, 250, 100, 50, 25, 15, 10, 9, 8, 7, 6, 5, 4, 3, or 2 genes. In one embodiment, the plurality of polynucleotide probes comprises polynucleotide probes for detecting at least 4 or 5 of the aforementioned genes, wherein the plurality of polynucleotide probes contains polynucleotide probes for no more than 10 genes. The polynucleotide probes may be optionally labeled. The kit may optionally include polynucleotide primers for amplifying a portion of the mRNA transcripts from at least 3, 4, 5, 6, 7, or 8 of the following human genes: PCA3, ALOX15, AMACR, CDH19, OR51E2/PSGR, F5, FZD8, and CLDN3.

Yet another embodiment is directed to a kit for diagnosing or prognosing prostate cancer in a patient of African descent, the kit comprising a plurality of polynucleotide probes for detecting at least 3, 4, 5, 6, or 7 of the following human genes: COL10A1, HOXC4, ESPL1, MMP9, ABCA13, PCDHGA1, and AGSK1, wherein the plurality of polynucleotide probes contains polynucleotide probes for no more than 500, 250, 100, 50, 25, 15, 10, 9, 8, 7, 6, 5, 4, 3, or 2 genes. In one embodiment, the plurality of polynucleotide probes comprises polynucleotide probes for detecting at least 4 or 5 of the aforementioned genes, wherein the plurality of polynucleotide probes contains polynucleotide probes for no more than 10 genes. The polynucleotide probes may be optionally labeled. The kit may optionally include polynucleotide primers for amplifying a portion of the mRNA transcripts from at least 3, 4, 5, 6, or 7 of the following human genes: COL10A1, HOXC4, ESPL1, MMP9, ABCA13, PCDHGA1, and AGSK1.

The kit for diagnosing or prognosing prostate cancer may also comprise antibodies. Thus, in one embodiment, the kit for diagnosing or prognosing prostate cancer comprises a plurality of antibodies for detecting at least 3, 4, 5, 6, 7, 8, 9, 10, 11, 12, 13, 14, or 15 of the polypeptides encoded by the following human genes: DLX1, NKX2-3, CRISP3, PHGR1, THBS4, AMACR, GAP43, FFAR2, GCNT1, SIM2, STX19, KLB, APOF, LOC283177, and TRPM, wherein the plurality of antibodies contains antibodies for no more than 500, 250, 100, 50, 25, 15, 10, 9, 8, 7, 6, 5, 4, 3, or 2 polypeptides. In one embodiment, the plurality of antibodies comprises antibodies for detecting at least 4 or 5 of the polypeptides encoded by the aforementioned genes and wherein the plurality of antibodies contains antibodies for no more than 10 polypeptides. The antibodies may be optionally labeled.

In another embodiment, the kit for diagnosing or prognosing prostate cancer in a patient of Caucasian descent comprises a plurality of antibodies for detecting at least 3, 4, 5, 6, 7, or 8 of the polypeptides encoded by the following human genes: PCA3, ALOX15, AMACR, CDH19, OR51E2/PSGR, F5, FZD8, and CLDN3, wherein the plurality of antibodies contains antibodies for no more than 500, 250, 100, 50, 25, 15, 10, 9, 8, 7, 6, 5, 4, 3, or 2 polypeptides. In one embodiment, the plurality of antibodies comprises antibodies for detecting at least 4 or 5 of the polypeptides encoded by the aforementioned genes and wherein the plurality of antibodies contains antibodies for no more than 10 polypeptides. The antibodies may be optionally labeled.

In yet another embodiment, the kit for diagnosing or prognosing prostate cancer in a patient of African descent comprises a plurality of antibodies for detecting at least 3, 4, 5, 6, or 7 of the polypeptides encoded by following human genes: COL10A1, HOXC4, ESPL1, MMP9, ABCA13, PCDHGA1, and AGSK1, wherein the plurality of antibodies contains antibodies for no more than 500, 250, 100, 50, 25, 15, 10, 9, 8, 7, 6, 5, 4, 3, or 2 polypeptides. In one embodiment, the plurality of antibodies comprises antibodies for detecting at least 4 or 5 of the polypeptides encoded by the aforementioned genes and wherein the plurality of antibodies contains antibodies for no more than 10 polypeptides. The antibodies may be optionally labeled.

In another aspect, the kit for diagnosing or prognosing prostate cancer may comprise polypeptide probes that can be used, for example, in spectrometry methods, such as mass spectrometry. Thus, in one embodiment, the kit for diagnosing or prognosing prostate cancer comprises a plurality of polypeptide probes for detecting at least 3, 4, 5, 6, 7, 8, 9, 10, 11, 12, 13, 14, or 15 of the polypeptides encoded by the following human genes: DLX1, NKX2-3, CRISP3, PHGR1, THBS4, AMACR, GAP43, FFAR2, GCNT1, SIM2, STX19, KLB, APOF, LOC283177, and TRPM, wherein the plurality of polypeptide probes contains polypeptide probes for no more than 500, 250, 100, 50, 25, 15, 10, 9, 8, 7, 6, 5, 4, 3, or 2 polypeptides. In one embodiment, the plurality of polypeptide probes comprises polypeptide probes for detecting at least 4 or 5 of the polypeptides encoded by the aforementioned genes and wherein the plurality of polypeptide probes contains polypeptide probes for no more than 10 polypeptides. The polypeptide probes may be optionally labeled.

In another embodiment, the kit for diagnosing or prognosing prostate cancer in a patient of Caucasian descent comprises a plurality of polypeptide probes for detecting at least 3, 4, 5, 6, 7, or 8 of the polypeptides encoded by the following human genes: PCA3, ALOX15, AMACR, CDH19. OR51E2/PSGR, F5, FZD8, and CLDN3, wherein the plurality of polypeptide probes contains polypeptide probes for no more than 500, 250, 100, 50, 25, 15, 10, 9, 8, 7, 6, 5, 4, 3, or 2 polypeptides. In one embodiment, the plurality of polypeptide probes comprises polypeptide probes for detecting at least 4 or 5 of the polypeptides encoded by the aforementioned genes and wherein the plurality of polypeptide probes contains polypeptide probes for no more than 10 polypeptides. The polypeptide probes may be optionally labeled.

In yet another embodiment, the kit for diagnosing or prognosing prostate cancer in a patient of African descent comprises a plurality of polypeptide probes for detecting at least 3, 4, 5, 6, or 7 of the polypeptides encoded by following human genes: COL10A1, HOXC4, ESPL1, MMP9, ABCA13, PCDHGA1, and AGSK1, wherein the plurality of polypeptide probes contains polypeptide probes for no more than 500, 250, 100, 50, 25, 15, 10, 9, 8, 7, 6, 5, 4, 3, or 2 polypeptides. In one embodiment, the plurality of polypeptide probes comprises polypeptide probes for detecting at least 4 or 5 of the polypeptides encoded by the aforementioned genes and wherein the plurality of polypeptide probes contains polypeptide probes for no more than 10 polypeptides. The polypeptide probes may be optionally labeled.

In one embodiment, a kit includes instructional materials disclosing methods of use of the kit contents in a disclosed method. The instructional materials may be provided in any number of forms, including, but not limited to, written form (e.g., hardcopy paper, etc.), in an electronic form (e.g., computer diskette or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes, labeling compounds, and the like. Such kits and appropriate contents are well known to those of skill in the art. The kit can also include a reference or control sample. The reference or control sample can be a biological sample or a data base.

As noted above, the polynucleotide or polypeptide probes and antibodies described in this application are optionally labeled with a detectable label. Any detectable label used in conjunction with probe or antibody technology, as known by one of ordinary skill in the art, can be used. In a particular embodiment, the probe is labeled with a detectable label selected from the group consisting of: a fluorescent label, a chemiluminescent label, a quencher, a radioactive label, biotin, mass tags and/or gold.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Comparative Genomic DNA Analysis

A comparative full genome analysis was conducted using primary prostate tumors and corresponding normal tissue (blood) in a cohort of seven AA and seven CA CaP patients (28 specimens). The cohort was selected based on the following criteria: primary treatment radical prostatectomy, no neo-adjuvant treatment. Gleason grade 3+3 and 3+4 (representing the majority of PSA-screened CaP at diagnosis/primary treatment), frozen tumor tissue with 80% or more tumor cell content, dissected tumor tissue yielding over 2 μg high molecular weight genomic DNA, availability of corresponding blood genomic DNA and patient clinico-pathological data.

28 samples were sent to Illumina Inc. (UK) for sequencing. Sequences from tumor samples were mapped to the reference genome using Illumina's ELAND alignment algorithm. Sequencing reported good coverage (average 37). Variant calling for single nucleotide polymorphisms (SNPs), small insertions and deletions (InDels), copy number variants (CNVs), and structural variants (SVs) was performed concurrently using the Strelka algorithm. All established CaP mutations (TMPRSS2/ERG, SPOP, CHD1, and PTEN) were identified at expected frequencies in this cohort.

Thirty one genes (including known mutations) with SNV, CNV or InDel somatic mutations in at least two of 14 patients were identified: AC091435.2; APC; ASMTL; ASMTL-AS1; CDC73; CHD1; CSF2RA; EYS; FRG1; FRG1B; HK2; IL3RA; KLLN; LIPF; LOC100293744; MT-ATP6; MT-BD4; MT-CO1; MT-CYB; MT-ND2; MT-ND3; MUC16; MUC6; NOX3; PDHA2; PTEN; SLC25A6; SLC9B1; SPOP; TRAV20; and USH2A.

The mutations did not appear to exhibit association with any specific group (AA−, AA+, CA−, CA+). However, the absence of PTEN deletions in AA patients was unexpected. Unequivocal PTEN deletion was detected in two CA cases with lesser apparent PTEN deletion in three additional CA cases, indicating the potential exclusivity of PTEN deletions in CA cases.

### Example 2

### Comparative RNA Analysis

To complement the genomic DNA analysis, RNA-Seq analysis was performed in the same cohort of prostate tumor samples. RNA-Seq technology has the ability to interrogate multiple aspects of the transcriptome including gene fusion, gene and transcript expression. Surrounding normal tissue was collected from 4 of the 14 patients. Two of the normal tissue samples were from AA men and two were from CA men.

RNA samples were shipped to Expression Analysis (Durham, N.C.) for transcriptome sequencing. The details of sequencing statistics are as follows: Sequencing type: paired-end, average read length of each sample: 50 nt, average read quality: 37, number of reads: approximately 31 million in each sample.

Raw reads from expression analysis were obtained in fastq format for each sample. These files contain all sequences passing Illumina's purity filter and per-base quality score as defined by Illumina phred metric. These raw reads were filtered for low quality reads (quality score<20), artifact/duplicate sequences and adapter sequences prior to actual analysis.

The human reference genome (hg19) used for mapping in this analysis was downloaded from UCSC website. Clean Paired end reads were aligned to the hg19 reference genome using TopHat software version 2.0.8 (a free open source tool available for mapping), and for each read no more than 2 mismatches were allowed in the alignment. TopHat maps reads to the reference genome using Bowtie, an ultra high-throughput short read aligner. Software outputs numerous files for further analysis: mapped reads, fusion junction, splice junction, insertions and deletions files.

Aligned reads were assembled to transcripts using Cufflinks, an open source tool that assembles transcripts, estimates their abundances, and tests for differential expression and regulation in RNA-Seq samples. Cufflinks calculates the expression level of gene depending on all the known splice variants/isoform of that gene.

Cufflinks measures transcript and Gene abundance levels in Fragments per Kilobase of transcript per Million mapped reads (FPKM). FPKM formula is defined as below:

FPKM=C/LN

C is the number of mappable reads in the feature (transcript, exon), L is the length in feature (in kb) and N is the total number of mappable reads in the feature (in millions). The FPKM normalization method eliminates discrepancies of gene lengths and sequencing for comparing the differences in gene expression between samples.

The Cuffdiff program, included in the Cufflinks package, calculates differential expression between CaP tumor and normal samples and includes a p-value for each observed change in expression between samples. Cuffdiff allows inputting multiple sample files based on the experimental condition. Gene and transcript expression levels are reported in tabular format and these files contain gene information, log 2 scale fold change for each gene, P values and false discovery rate (FDR). Pathway analysis and Gene Ontology Biological Processes on statistically significant genes was performed with Genomatix Pathway Analysis Software.

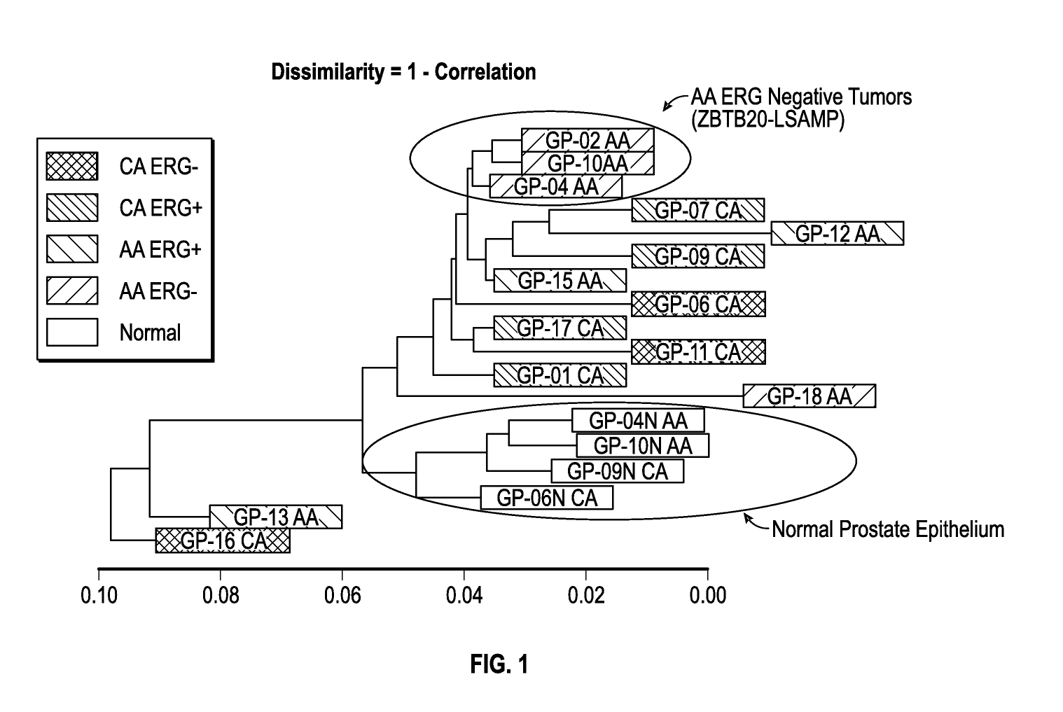

Hierarchical clustering (performed using R package) was used to group samples based on their expression levels. FIG. 1. Clustering of all 18 samples (14 tumors and 4 normal) indicate a clear demarcation between tumor and normal samples. However, most of the tumor samples were not clustered according to AA and CA groups. Interestingly, three out of four fusion negative AA samples clustered in to one group, and based on the patient follow up studies, two of the 3 patients developed metastasis (the only two metastasis in this cohort), and the third had biochemical recurrence.

Gene expression profiles were obtained from 14 tumor (7 AA and 7 CA) and 4 normal (2 AA and 2 CA) samples. A limitation for the comparison analysis between tumor and normal samples was the availability of normal samples for all tumor samples. Hence, in the current analysis two normal samples within each group were pooled together and the average value was compared to their respective groups.

In the initial analysis, gene expression profiles for each patient were generated by comparing tumor with normal sample within each group. Statistically significant genes were extracted using fold change (tumor/normal ratios), at least 2 fold over/under expressed in tumors and P-value<0.05. 101 genes and 180 genes were statistically significant (2 fold p-value<0.05) in African- and Caucasian-American groups respectively. Few of the prostate cancer literature associated genes in African-American list included CRISP3, SIM2, THBS4 and MMP9; Caucasian-American list included AMACR, APOF, CRISP3, OR51E2 (PSGR), SIM2 and THBS4.

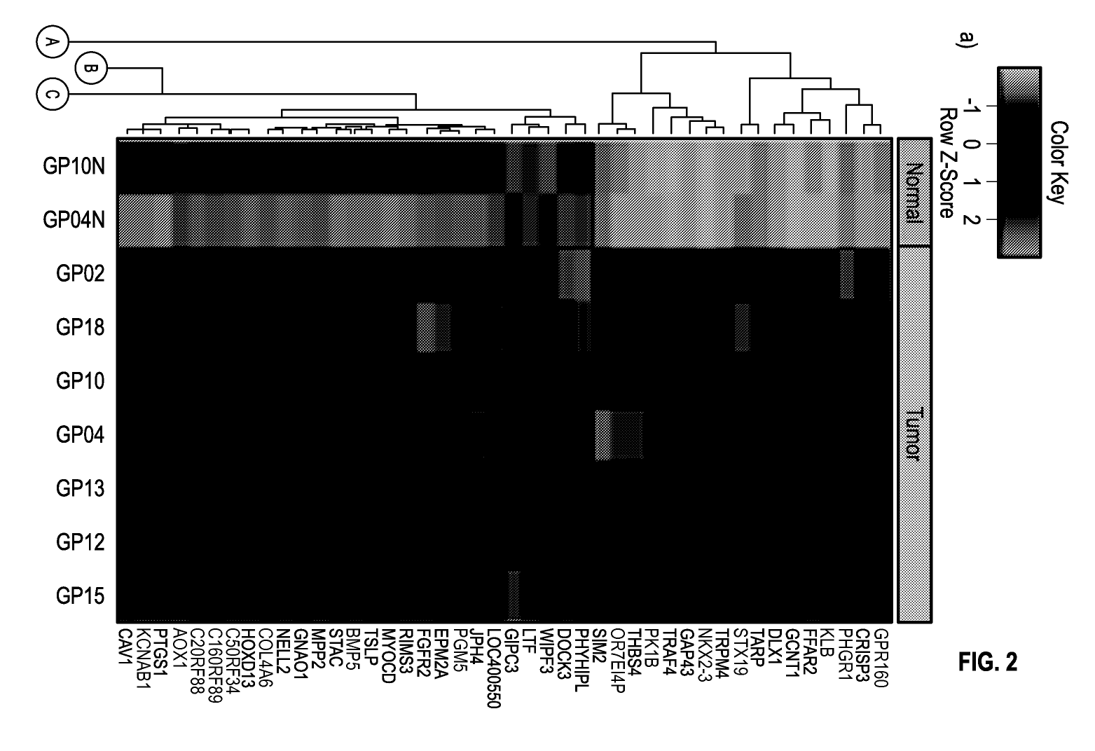

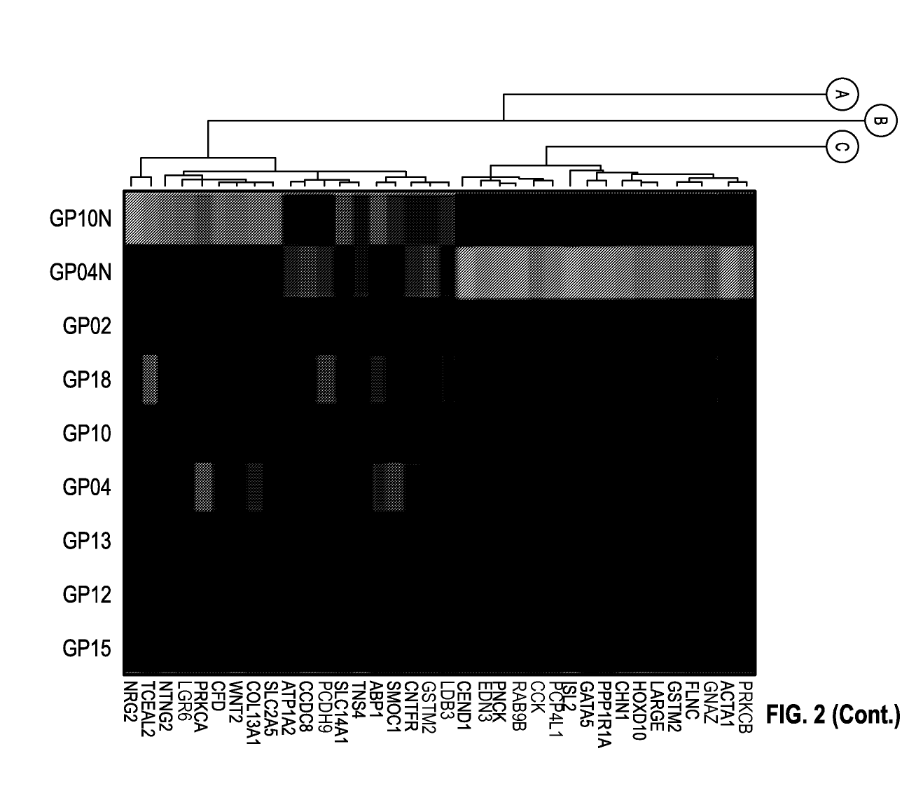

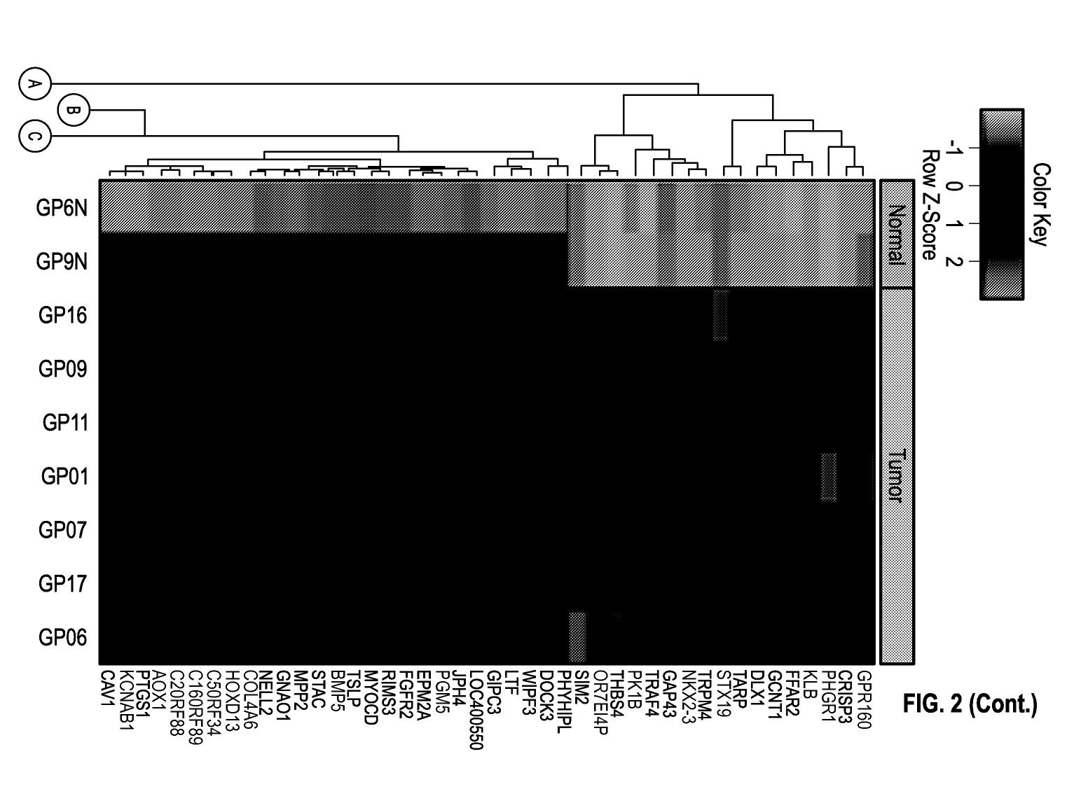

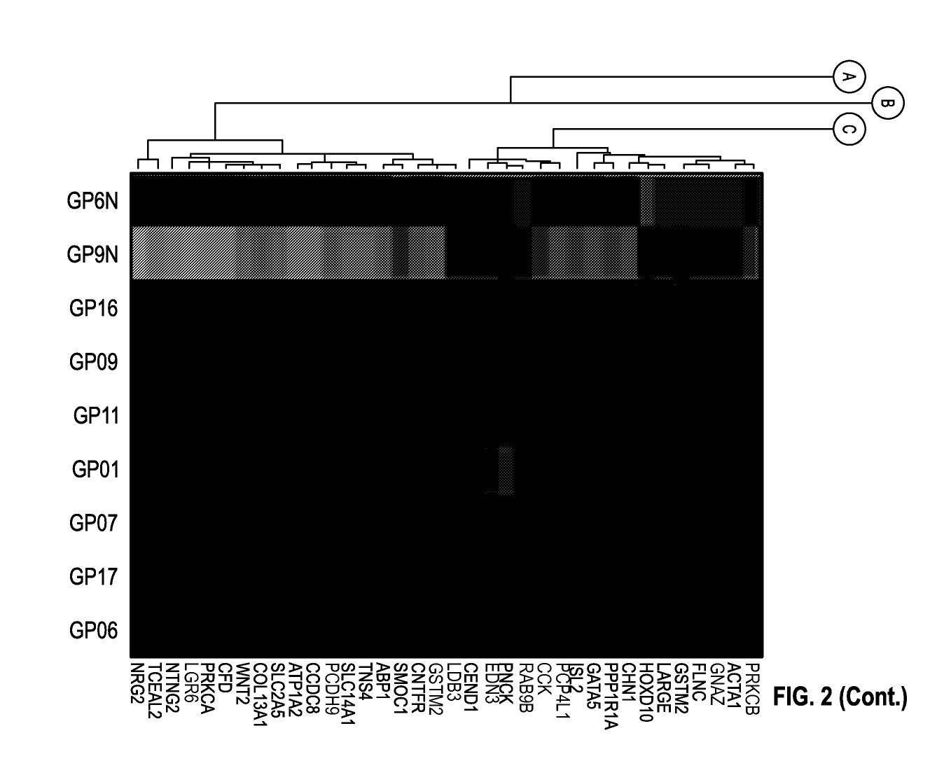

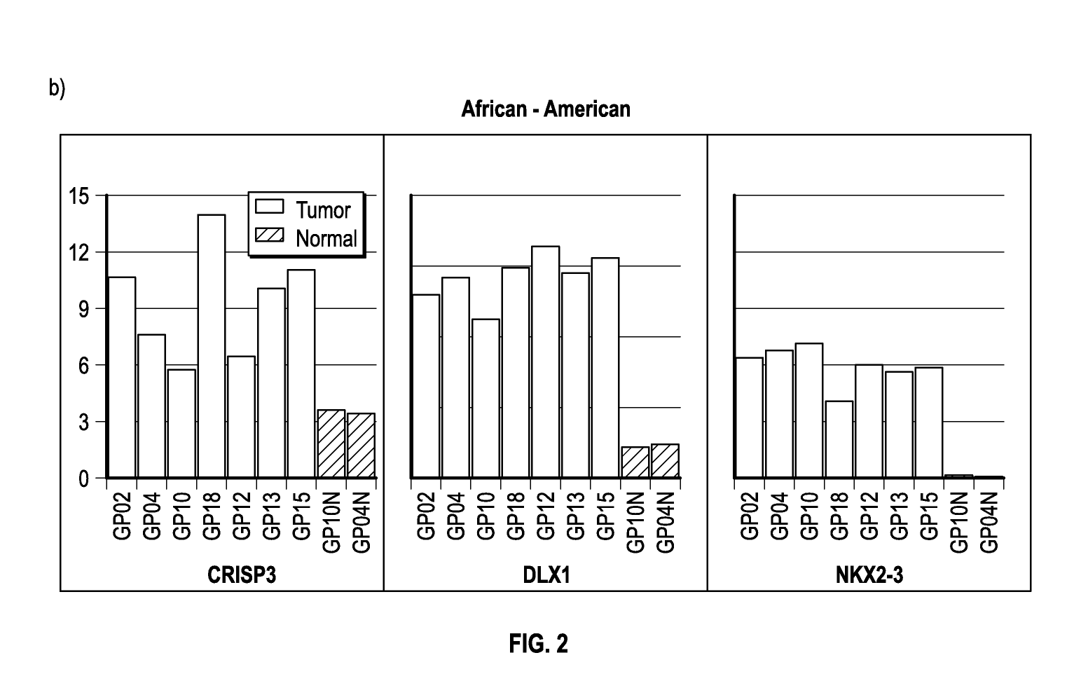

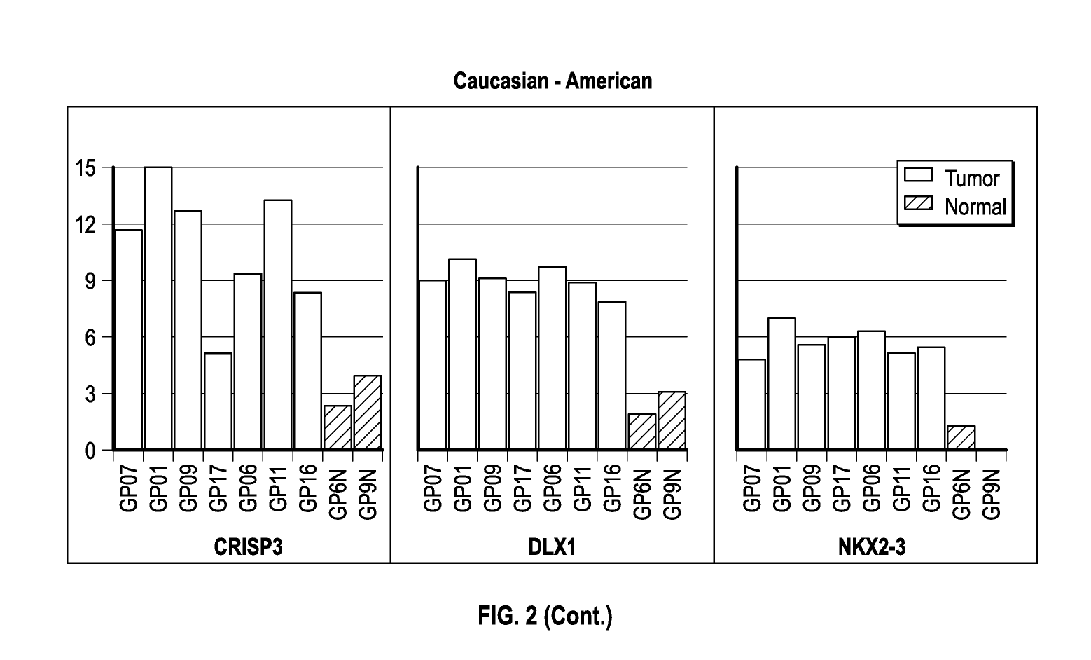

Comparison of AA gene list to CA gene lists showed 84 genes to be common in both the ethnic groups. The list of common genes included a few well studied genes in prostate cancer like AMACR, CRISP3 and SIM2, DLX1, NKX3-2 and CRISP3 were the top over expressed genes in this list. FIG. 2. The most consistently overexpressed genes in both ethnic groups were DLX1 and NKX2-3. The top 15 over expressed genes in both AA and CA prostate tumors are listed in Table 5 (ranked by fold change).

TABLE 5

| Gene Symbol | AA | CA |
|---|---|---|
| DLX1 | 168.76 | 94.24 |
| NKX2-3 | 128.58 | 49.14 |
| CRISP3* | 128.08 | 711.14 |
| PHGR1 | 44.04 | 100.24 |
| THBS4 | 17.37 | 18.87 |
| AMACR* | 11.89 | 25 |
| GAP43 | 7.19 | 7.99 |
| FFAR2 | 7.03 | 8.54 |
| GCNT1 | 6.62 | 15.23 |
| SIM2* | 6.41 | 9.68 |
| STX19 | 5.98 | 7.67 |
| KLB | 5.07 | 5.91 |
| APOF | 5.04 | 19.42 |
| LOC283177 | 4.86 | 7 |
| TRPM4 | 4.35 | 6.48 |

*Known gene alternation in prostate cancer

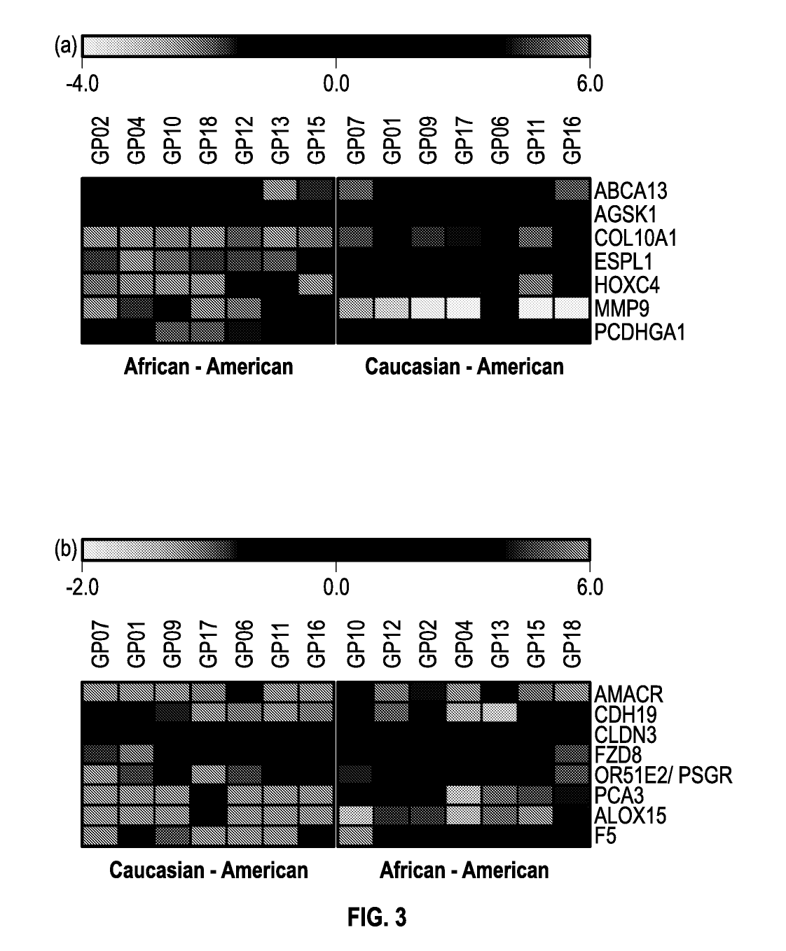

Similarly, tumor/normal ratios of CA group (180 genes) were compared to the tumor/normal ratios in AA group. This gene list revealed that some of the well-studied prostate cancer genes, such as, PCA3 (10-fold), PSGR (5-fold) and AMACR (2-fold) were over expressed in the CA group as compared to the AA group (FIG. 3B). Additionally, gene expression levels of TMPRSS2/ERG fusion positive samples were compared with fusion negative samples. CRISP3, GLDC, and TDRD1 were the top differentially expressed genes in TMPRSS2/ERG fusion positive samples, while COL2A1 and PLA2G7 were the top differentially expressed genes in TMPRSS2/ERG fusion negative samples. The top differentially expressed genes in prostate tumors of the CA group as compared to the AA group are set forth in Table 6.

TABLE 6

| Gene Symbol | CA | AA |
|---|---|---|
| PCA3* | 94.76 | 6.09 |
| ALOX15 | 79.68 | 9.66 |
| AMACR* | 25 | 11.89 |
| CDH19 | 13.73 | 1.43 |
| OR51E2/PSGR | 10.79 | 2.8 |
| F5 | 8.89 | 4.16 |
| FZD8 | 7.72 | 3.08 |
| CLDN3 | 5.28 | 2.58 |

*current prostate cancer diagnostic markers

The tumor/normal ratios of differentially expressed gene list in AA group (101 genes) were compared to the tumor/normal ratios in CA group to evaluate AA race specific gene expression trend. The heatmap in FIG. 3A shows genes that were consistently up-regulated in the AA group and simultaneously down-regulated (or no change of expression) in the CA group. In this list, MMP9 was the top gene which was found to very strongly up-regulated in the AA group but down-regulated in the CA group. The top differentially expressed genes in prostate tumors of the AA group as compared to the CA group are set forth in Table 7.

TABLE 7

| Gene Symbol | AA | CA |
|---|---|---|
| COL10A1 | 539.86 | 16.81 |
| HOXC4 | 72.06 | 13.13 |
| ESPL1 | 35.49 | 1.92 |
| MMP9 | 32.23 | 0.27 |
| ABCA13 | 22.65 | 2.02 |
| PCDHGA1 | 15.15 | 1.82 |
| AGSK1 | 6.09 | 0.98 |

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

The following references are cited in the application and provide general information on the field of the invention and provide assays and other details discussed in the application. The following references are incorporated herein by reference in their entirety.
1. Siegel, R.; Naishadham, D.; Jemal, A. Cancer statistics. CA Cancer J. Clin. 2013, 63, 11-30.
2. Chornokur, G.; Dalton, K.; Borysova, M. E.; Kumar, N. B. Disparities at presentation, diagnosis, treatment, and survival in African American men affected by prostate cancer. Prostate 2011, 71, 985-997.
3. Schwartz, K.; Powell. L J.; Underwood, W., 3rd; George, J.; Yee, C.; Banerjee, M. Interplay of race, socioeconomic status, and treatment on survival of patients with prostate cancer. Urology 2009, 74, 1296-1302.
4. Major, J. M.; Oliver, M. N.; Doubeni, C. A.; Hollenbeck, A. R.; Graubard, B. I.; Sinha. R. Socioeconomic status, healthcare density, and risk of prostate cancer among African American and Caucasian men in a large prospective study. Cancer Causes Control 2012, 23, 1185-1191.
5. Sridhar, G.; Masho, S. W.; Adera, T.; Ramakrishnan, V.; Roberts, J. D. Do African American men have lower survival from prostate cancer compared with White men? A meta-analysis. Am. J Mens. Health 2010, 4, 189-206.
6. Cullen, J.; Brassell, S.; Chen, Y.; Porter, C.; L'Esperance, J.; Brand, T.; McLeod, D. G. Racial/ethnic patterns in prostate cancer outcomes in an active surveillance cohort. Prostate Cancer 2011, 2011, doi:10.1155/2011/234519.
7. Berger, A. D.; Satagopan, J.; Lee, P.; Taneja, S. S.; Osman. I. Differences in clinicopathologic features of prostate cancer between black and white patients treated in the 1990s and 2000s. Urology 2006, 67, 120-124.
8. Kheirandish, P.; Chinegwundoh, F. Ethnic differences in prostate cancer. Br. J. Cancer 2011, 105, 481-485.
9. Odedina, F. T.; Akinremi, T. O.; Chinegwundoh, F.; Roberts, R.; Yu, D.; Reams, R. R.; Freedman, M. L.; Rivers, B.; Green, B. L.; Kumar, N. Prostate cancer disparities in black men of African descent: A comparative literature review of prostate cancer burden among black men in the United States, Caribbean, United Kingdom, and West Africa. Infect. Agents Cancer 2009, 4, doi:10.1186/1750-9378-4S1-S2.
10. Heath, E. I.; Kattan, M. W.; Powell, I. J.; Sakr, W.; Brand, T. C.; Rybicki, B. A.; Thompson. I. M.; Aronson, W. J.; Terris, M. K.; Kane, C. J.; et al. The effect of race/ethnicity on the accuracy of the 2001 Partin Tables for predicting pathologic stage of localized prostate cancer. Urology 2008, 71, 151-155.
11. Moul, J. W.; Sesterhenn, I. A.; Connelly, R. R.; Douglas, T.; Srivastava, S.; Mostofi, F. K.; McLeod, D. G. Prostate-specific antigen values at the time of prostate cancer diagnosis in African-American men. JAMA 1995, 274, 1277-1281.
12. Tewari, A.; Horninger, W.; Badani, K. K.; Hasan, M.; Coon, S.; Crawford, E. D.; Gamito. E. J.; Wei, J.; Taub, D.; Montie, J.; et al. Racial differences in serum prostate-specific (PSA) doubling time, histopathological variables and long-term PSA recurrence between African-American and white American men undergoing radical prostatectomy for clinically localized prostate cancer. BJU Int. 2005, 96, 29-33.
13. Wallace, T. A.; Prueitt, R. L.; Yi, M.; Howe, T. M.; Gillespie J. W.; Yfantis, H. G.; Stephens, R. M.; Caporaso, N. E.; Loffredo, C. A.; Ambs, S. Tumor immunobiological differences in prostate cancer between African-American and Caucasian-American men. Cancer Res. 2008, 68, 927-936.
14. Prensner, J. R.; Rubin, M. A.; Wei, J. T.; Chinnaiyan, A. M. Beyond PSA: The next generation of prostate cancer biomarkers. Sci. Transl. Med 2012, 4, doi:10.1126/scitranslmed.3003180.
15. Rubin, M. A.; Maher, C. A.; Chinnaiyan, A. M. Common gene rearrangements in prostate cancer. J. Clin. Oncol. 2011, 29, 3659-3668.
16. Sreenath, T. L.; Dobi, A.; Petrovics, G.; Srivastava, S. Oncogenic activation of ERG: A predominant mechanism in prostate cancer. J. Carcinog. 2011, 11, 10-21.
17. Petrovics, G.; Liu, A.; Shaheduzzaman, S.; Furasato, B.; Sun, C.; Chen, Y.; Nau, M. Ravindranath, L.; Chen, Y.; Dobi, A.; et al. Frequent overexpression of ETS-related gene-1 (ERG1) in prostate cancer transcriptome. Oncogene 2005, 24, 3847-3852.
18. Tomlins, S. A.; Rhodes, D. R.; Perner, S.; Dhanasekaran, S. M.; Mehra, R.; Sun, X. W.; Varambally, S.; Cao, X.; Tchinda, J.; Kuefer, R.; et al. Recurrent fusion of TMPRSS2 and ETS transcription factor genes in prostate cancer. Science 2005, 310, 644-648.
19. Magi-Galluzzi, C.; Tsusuki, T.; Elson, P.; Simmerman, K.; LaFarque, C.; Esqueva. R.; Klein, E.; Rubin, M. A.; Zhou, M. TMPRSS2-ERG gene fusion prevalence and class are significantly different in prostate cancer of Caucasian, African-American and Japanese patients. Prostate 2011, 71, 489-497.
20. Rosen, P.; Pfister, D.; Young, D.; Petrovics, G.; Chen, Y.; Cullen, J.; Bohm, D.; Perner, S.; Dobi, A.; McLeod, D. O.; et al. Differences in frequency of ERG oncoprotein expression between index tumors of Caucasian and African American patients with prostate cancer. Urology 2012, 80, 749-753.
21. Hu, Y.; Dobi, A.; Sreenath, T.; Cook, C.; Tadase, A. Y.; Ravindranath, L.; Cullen, J.; Furusato, B.; Chen, Y.; Thanqapazham, R. L.; et al. Delineation of TMPRSS2-ERG splice variants in prostate cancer. Clin. Cancer Res. 2008, 14, 4719-4725.
22. Gary K Geiss, et al. (2008) Direct multiplexed measurement of gene expression with color-coded probe pairs, Nature Biotechnology 26:317-25.
23. Paolo Fortina and Saul Surrey, (2008) Digital mRNA Profiling, Nature Biotechnology 26:317-25.
24. Farrell J. Petrovics G. McLeod D G, Srivastava S.: Genetic and molecular differences in prostate carcinogenesis between African American and Caucasian American men. International Journal of Molecular Sciences. 2013; 14(8):15510-31.
25. Rodriquez-Suarez et al., Urine as a source for clinical proteome analysis: From discovery to clinical application, Biochimica et Biophysica Acta (2013).
26. Shi et al., Antibody-free, targeted mass-spectrometric approach for quantification of proteins at low picogram per milliliter levels in human plasma/serum. PNAS, 109(38):15395-15400 (2012).
27. Elentihoba-Johnson and Lim, Fusion peptides from oncogenic chimeric proteins as specific biomarkers of cancer, Mol Cell Proteomics, 12:2714 (2013).

1. A method of collecting data for use in diagnosing or prognosing prostate cancer in a patient, the method comprising:
a) detecting expression of a plurality of genes in a biological sample obtained from the patient, wherein the patient is of African descent and wherein the plurality of genes is selected because the patient is of African descent and comprises at least three of the following genes: COL10A1, HOXC4, ESPL1, MMP9, ABCA13, PCDHGA1, and AGSK1;
b) detecting expression of a plurality of genes in a biological sample obtained from the patient, wherein the patient is of Caucasian descent and wherein the plurality of genes is selected because the patient is of Caucasian descent and comprises at least four of the following genes: PCA3, ALOX15, AMACR, CDH19, OR51E2/PSGR, F5, FZD8, and CLDN; or
c) detecting expression of a plurality of genes in a biological sample obtained from the patent, wherein the plurality of genes comprises at least four of the following genes: DLX1, NKX2-3, CRISP3, PHGR1, THBS4, AMACR, GAP43, FFAR2, GCNT1, SIM2, STX19, KLB, APOF, LOC283177, and TRPM4.

2. The method of claim 1, further comprising a step of diagnosing or prognosing prostate cancer using the expression data obtained in step a), step b), or step c).

3. The method of claim 2, wherein overexpression of 1) at least three of the following genes: COL10A1, HOXC4, ESPL1, MMP9, ABCA13, PCDHGA1, and AGSK1; 2) at least four of the following genes: PCA3, ALOX15, AMACR, CDH19, OR51E2/PSGR, F5, FZD8, and CLDN3; or 3) at least four of the following genes: DLX1, NKX2-3, CRISP3, PHGR1, THBS4, AMACR, GAP43, FFAR2, GCNT1, SIM2, STX19, KLB, APOF, LOC283177, and TRPM4 as compared to a control sample or a threshold value indicates the presence of prostate cancer in the biological sample or an increased risk of developing prostate cancer.

4. The method of claim 1, further comprising detecting expression of an ERG gene in the biological sample.

5. The method of claim 1, wherein the biological sample is a tissue sample, a cell sample, a blood sample, a serum sample, or a urine sample.

6. The method of claim 1, wherein the biological sample comprises prostate cells or nucleic acids or polypeptides isolated from prostate cells.

7. The method of claim 1, wherein nucleic acid expression is detected in steps a), b), or c).

8. The method of claim 1, wherein polypeptide expression is detected in steps a), b), or c).

9. A kit for use in diagnosing or prognosing prostate cancer, the kit comprising a plurality of probes for detecting at least three of the following genes: COL10A1, HOXC4, ESPL1, MMP9, ABCA13, PCDHGA1, and AGSK1, wherein the plurality of probes contains probes for detecting no more than 500, 250, 100, 50, 25, 15, 10, 9, 8, 7, 6, 5, 4, 3, or 2 different genes.

10. A kit for use in diagnosing or prognosing prostate cancer, the kit comprising a plurality of probes for detecting at least four of the following genes: PCA3, ALOX15, AMACR, CDH19, OR51E2/PSGR, F5, FZD8, and CLDN3, wherein the plurality of probes contains probes for detecting no more than 500, 250, 100, 50, 25, 15, 10, 9, 8, 7, 6, 5, 4, 3, or 2 different genes.

11. A kit for use in diagnosing or prognosing prostate cancer, the kit comprising a plurality of probes for detecting at least four of the following genes: DLX1, NKX2-3, CRISP3, PHGR1, THBS4, AMACR, GAP43, FFAR2, GCNT1, SIM2, STX19, KLB, APOF, LOC283177, and TRPM4, wherein the plurality of probes contains probes for detecting no more than 500, 250, 100, 50, 25, 15, 10, 9, 8, 7, 6, 5, 4, 3, or 2 different genes.

12. The kit of claim 9, wherein the plurality of probes is selected from a plurality of oligonucleotide probes, a plurality of antibodies, or a plurality of polypeptide probes.

13. The kit of claim 9, wherein the plurality of probes contains probes for detecting no more than 250, 100, 50, 25, 15, 10, or 5 different genes.

14. The kit of claim 9, wherein the plurality of probes are attached to the surface of an array.

15. The kit of claim 14, wherein the array comprises no more than 500, 250, 100, 50, 25, 15, or 10 addressable elements.

16. The kit of claim 9, wherein the plurality of probes are labeled.

17. The kit of claim 9, wherein the plurality of probes further comprises a probe for detecting an ERG gene.

18. A method of obtaining a gene expression profile in a biological sample, the method comprising:
a) incubating the array of claim 14 with the biological sample, wherein the biological sample is obtained from a patient of African descent; and
b) measuring the expression level of at least three of the following genes: COL10A1, HOXC4, ESPL1, MMP9, ABCA13, PCDHGA1, and AGSK1 to obtain the gene expression profile.

19. A method of obtaining a gene expression profile in a biological sample, the method comprising:
a) incubating the array of claim 35 with the biological sample, wherein the biological sample is obtained from a patient of Caucasian descent; and
b) measuring the expression level of at least four of the following genes: PCA3, ALOX15, AMACR, CDH19, OR51E2/PSGR, F5, FZD8, and CLDN3 to obtain the gene expression profile.

20. A method of obtaining a gene expression profile in a biological sample, the method comprising:
a) incubating the array of claim 36 with the biological sample; and
b) measuring the expression level of at least four of the following genes: DLX1, NKX2-3, CRISP3, PHGR1, THBS4, AMACR, GAP43, FFAR2, GCNT1, SIM2, STX19, KLB, APOF, LOC283177, and TRPM4 to obtain the gene expression profile.

21. The method of claim 18, wherein the biological sample is a tissue sample, a cell sample, a blood sample, a serum sample, or a urine sample.

22. The method of claim 18, wherein the biological sample comprises nucleic acids or polypeptides isolated from prostate cells.

23. The method of claim 18, wherein the measuring step comprises measuring nucleic acid expression levels.

24. The method of claim 18, wherein the measuring step comprises measuring polypeptide expression levels.

25. The method of claim 18, wherein the measuring step further comprises measuring the expression level of an ERG gene.

26. A method of identifying a patient in need of prostate cancer treatment, wherein the patient is of African descent, the method comprising:
a) testing a biological sample from the patient for the overexpression of a plurality of genes, wherein the plurality of genes is selected because the patient is of African descent and comprises at least three of the following genes: COL10A1, HOXC4, ESPL1, MMP9, ABCA13, PCDHGA1, and AGSK1; and
b) identifying the patient as in need of prostate cancer treatment if one or more of the COL10A1, HOXC4, ESPL1, MMP9, ABCA13, PCDHGA1, and AGSK1 genes is overexpressed in the biological sample as compared to a control sample or a threshold value.

27. The method of claim 26, further comprising a step of treating the patient if the patient is identified as in need of prostate cancer treatment.

28. A method of identifying a patient in need of prostate cancer treatment, wherein the patient is of Caucasian descent, the method comprising:
a) testing a biological sample from the patient for the overexpression of a plurality of genes, wherein the plurality of genes is selected because the patient is of Caucasian descent and comprises at least four of the following genes: PCA3, ALOX15, AMACR, CDH19, OR51E2/PSGR, F5, FZD8, and CLDN3; and
b) identifying the patient as in need of prostate cancer treatment if one or more of the PCA3, ALOX15, AMACR, CDH19, OR51E2/PSGR, F5, FZD8, and CLDN3 genes is overexpressed in the biological sample as compared to a control sample or a threshold value.

29. The method of claim 28, further comprising a step of treating the patient if the patient is identified as in need of prostate cancer treatment.

30. A method of treating prostate cancer in a patient, wherein the patient is of African descent, the method comprising:
a) testing a biological sample from the patient for the expression of a plurality of genes, wherein the plurality of genes is selected because the patient is of African descent and comprises at least three of the following genes: COL10A1, HOXC4, ESPL1, MMP9, ABCA13, PCDHGA1, and AGSK1;
b) treating the patient if the testing in step a) reveals that the patient overexpresses as compared to a control sample or threshold value one or more of the following genes: COL10A1, HOXC4, ESPL1, MMP9, ABCA13, PCDHGA1, and AGSK1.

31. A method of treating prostate cancer in a patient, wherein the patient is of Caucasian descent, the method comprising:
a) testing a biological sample from the patient for the expression of a plurality of genes, wherein the plurality of genes is selected because the patient is of Caucasian descent and comprises at least four of the following genes: PCA3, ALOX15, AMACR, CDH19, OR51E2/PSGR, F5, FZD8, and CLDN3;
b) treating the patient if the testing in step a) reveals that the patient overexpresses as compared to a control sample or threshold value one or more of the following genes: PCA3, ALOX15, AMACR, CDH19, OR51E2/PSGR, F5, FZD8, and CLDN3.

32. The method of claim 28, further comprising testing the biological sample for expression of an ERG gene.

33. The method of claim 1, wherein the plurality of genes consists of no more than 100, 90, 80, 70, 60, 50, 40, 30, 25, 20, 15, 14, 13, 12, 11, 10, 9, 8, 7, 6, 5, 4, 3, or 2 genes.

34. (canceled)

35. The kit of claim 10, wherein the plurality of probes are attached to the surface of an array and the array comprises no more than 500, 250, 100, 50, 25, 15, or 10 addressable elements.

36. The kit of claim 11, wherein the plurality of probes are attached to the surface of an array and the array comprises no more than 500, 250, 100, 50, 25, 15, or 10 addressable elements.
